(19) United States
(12) Patent Application Publication
(10) Pub. No.: US 2004/0033581 A1
(43) Pub. Date: Feb. 19, 2004

(54) Dual condensation/epimerization domain in non-ribosomal peptide synthetase systems

(76) Inventors: Chris M. Farnet, Outremont (CA); Alfredo Staffa, Saint Laurent (CA)

Correspondence Address: Pandiscio & Pandiscio, 470 Totten Pond Road, Waltham, MA, 02154, US

(21) Appl. No.: 10/417,700

(22) Filed: Apr. 17, 2003

Related U.S. Application Data
(63) Continuation-in-part of application No. 09/976,059, filed on Oct. 15, 2001.
(60) Provisional application No. 60/239,924, filed on Oct. 13, 2000.

(51) Int. Cl. C12Q 1/68; C07H 21/04; C12N 9/64; C12P 21/02; C12N 5/06

(52) U.S. Cl. 435/226; 435/6; 435/69.1; 435/320.1; 435/325; 536/23.2

(57) ABSTRACT

The present invention relates to domains of non-ribosomal peptide synthetases (NRPSs) that exhibit dual condensation and epimerization activities. The “dual condensation/epimerization NRPS domains” of the present invention allow for the incorporation of non-proteinogenic substrates, such as D-amino acids, into peptide products. These dual condensation/epimerization NRPS domains may further be used to modify the stereochemistry of synthesized peptides at selected amino acid sites.

### RELATED APPLICATIONS

The present application is a Continuation-in-part of application Ser. No. 09/976,059 filed on Oct. 15, 2001 which claims priority from application Ser. No. 60/239,924 filed on Oct. 13, 2000. The present application further claims the benefit of application Ser. No. 60/372,790 filed on Apr. 17, 2002. The full disclosure of each of these applications is incorporated herein by reference.

### FIELD OF THE INVENTION

The present invention relates to non-ribosomal peptide synthetases (NRPSs). More specifically, the present invention concerns domains of NRPSs that exhibit dual condensation and epimerization activities allowing for the incorporation of non-proteinogenic substrates, such as D-amino acids, into peptide products. These dual condensation/epimerization NRPS domains may further be used to modify the stereochemistry of synthesized peptides at selected amino acid sites.

### BACKGROUND

Many low molecular weight peptides produced by microorganisms are synthesized non-ribosomally on large multifunctional proteins termed non-ribosomal peptide synthetases (NRPSs). NRPSs are modular proteins that consist of one or more polyfunctional polypeptides each of which is made up of modules. The amino- to carboxy-terminal order and specificities of the individual modules correspond to the sequential order and identity of the amino acid residues of the peptide product. Each NRPS module recognizes a specific amino acid substrate and catalyzes the stepwise condensation to form a growing peptide chain. The identity of the amino acid recognized by a particular unit can be determined by comparison with other units of known specificity. In many peptide synthetases, there is a strict correlation between the order of repeated units in a peptide synthetase and the order in which the respective amino acids appear in the peptide product, making it possible to correlate peptides of known structure with putative genes encoding their synthesis, as demonstrated by the identification of the mycobactin biosynthetic gene cluster from the genome of Mycobacterium tuberculosis.

The modules of a peptide synthetase are composed of smaller units or “domains” that each carry out a specific role in the recognition, activation, modification and joining of amino acid precursors to form the peptide product. One type of domain, the adenylation (A) domain, is responsible for selectively recognizing and activating the amino acid that is to be incorporated by a particular unit of the peptide synthetase. This activation step is ATP-dependent and involves the transient formation of an amino-acyl-adenylate. The activated amino acid is covalently attached to the peptide synthetase through another type of domain, the thiolation (T) domain, that is generally located adjacent to the A domain. The T domain is post-translationally modified by the covalent attachment of a phosphopantetheinyl prosthetic arm to a conserved serine residue. The activated amino acid substrates are tethered onto the non-ribosomal peptide synthetase via a thioester bond to the phosphopantetheinyl prosthetic arm of the respective T domains. Amino acids joined to successive units of the peptide synthetase are subsequently covalently linked together by the formation of amide bonds catalyzed by another type of domain, the condensation (C) domain. NRPS modules can also occasionally contain additional functional domains that carry out auxiliary reactions, the most common being epimerization of an amino acid substrate from the L- to the D- form. This reaction is catalyzed by a domain referred to as an epimerization (E) domain that is generally located adjacent to the T domain of a given NRPS module. Thus, a typical NRPS module has the following domain organization: C-A-T-(E).

Product assembly by NRPSs involves three distinct phases, namely chain initiation, chain elongation, and chain termination. Polypeptide chain initiation is carried out by specialized modules termed “starter modules” that comprise an A domain and a T domain. Elongation modules have, in addition, a C domain that is located upstream of the A domain. Elongation domains cannot initiate peptide bond formation due to interference by the C domain. The peptide intermediates are covalently tethered to the NRPS during translocations as an elongating series of acyl-S-enzymes. To release the mature peptide product from the NRPS, the terminal acyl-S-enzyme bond must be broken. This process is the chain termination step and is usually catalyzed by a C-terminal thioesterase (TE) domain. Thioesterase-mediated release of the mature peptide from the NRPS enzyme involves the transient formation of an acyl-O-TE intermediate that is then hydrolyzed or hydrolyzed and concomitantly cyclized to release the mature peptide.

### SUMMARY OF THE INVENTION

We have discovered and isolated nucleic acid and polypeptide sequences for specialized condensation domains that direct the incorporation of non-proteinogenic substrates, such as D-amino acids (and occasionally non-chiral amino acids) into peptide products synthesized by non-ribosomal peptide synthetase systems that lack canonical epimerization domains (referred to herein and in U.S. Ser. No. 60/372,790 as E-less NRPSs).

In one aspect, the invention provides an isolated polynucleotide encoding a dual condensation/epimerization NRPS domain, said polynucleotide encodes a polypeptide having at least 45% sequence identity to SEQ ID NO: 139. Certain embodiments expressly exclude one or more sequences, in particular the nucleotide sequence corresponding to the C-domains of NRPS protein of GenBank accession no. AAC80285 (SEQ ID NOS: 18, 20, 22 and 24); the nucleotide sequence corresponding to the C-domains of NRPS protein of GenBank accession no. AAF99707, AAO72424 and AAO72425 (SEQ ID NOS: 26, 28, 30, 32, 34, 36, 38, 40, 42, 44, 46, 48, 50, 52, 54, 56, 58 and 60) and the nucleotide sequence corresponding to the C-domains of NRPS protein of GenBank accession no. CAD17793 (SEQ ID NOS: 62 and 64). Other sequences can be excluded without departing from the scope of the invention.

In a related aspect the invention provides an isolated polynucleotide comprising a sequence selected from the group consisting of: (a) a sequence selected from the group consisting of SEQ ID NOS: 2, 4, 6, 8,10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136 and 138; (b) a sequence that is complementary to (a); (c) a sequence which hybridizes to said sequence of (a) or (b) under conditions of high stringency; and (d) a sequence which has at least 60% or higher similarity to said sequence of (a), (b), or (c) as measured using the BLASTP 2.2.5 algorithm. Certain embodiments expressly exclude one or more sequences, in particular the nucleotide sequence corresponding to the C-domains of NRPS protein of GenBank accession no. AAC80285 (SEQ ID NOS: 18, 20, 22 and 24); the nucleotide sequence corresponding to the C-domains of NRPS protein of GenBank accession no. AAF99707, AAO72424 and AAO72425 (SEQ ID NOS: 26, 28, 30, 32, 34, 36, 38, 40, 42, 44, 46, 48, 50, 52, 56, 58 and 60) and the nucleotide sequence corresponding to the C-domains of NRPS protein of GenBank accession no. CAD17793 (SEQ ID NOS: 62 and 64). Other sequences can be excluded without departing from the scope of the invention.

In one embodiment, the isolated polynucleotide encoding a dual condensation/epimerization domain is derived from an organism of the actinomycetes taxon. In a further embodiment, the isolated polynucleotide encoding a dual condensation/epimerization NRPS domain resides in a gene locus selected from the group consisting of: the biosynthetic locus for ramoplanin from Actinoplanes sp. ATCC 33076; the biosynthetic locus for syringomycin from Pseudomonas syringae pv. syringae strain B301D; the biosynthetic locus for syringopeptin from Pseudomonas syringae pv. syringae strain B301D; the biosynthetic locus for a peptide natural product from Ralstonia solanacearum GMI1000; the biosynthetic locus for a lipopeptide natural product from Streptomyces aizunensis NRRL B-11277; the biosynthetic locus for a lipopeptide natural product from Streptomyces griseofuscus NRRL B-5429; the biosynthetic locus for a lipopeptide natural product from Kitasatosporia sp. ECO-03; the biosynthetic locus for a lipopeptide natural product from Streptomyces sp. ECO-38; the biosynthetic locus for a lipopeptide natural product from Streptomyces sp. ECO-59; the biosynthetic locus for a lipopeptide natural product from Streptomyces viridifaciens NRRL ISP-5239; the biosynthetic locus for a lipopeptide natural product from Actinomadura sp. ATCC 39334; and the biosynthetic locus for a lipopeptide natural product from Micromonospora chersina ATCC 53710. In still a further embodiment of the invention, the dual condensation/epimerization NRPS domain encoded by the isolated polynucleotide is involved in epimerization and condensation of amino acids in E-less NRPS systems. In one embodiment some dual condensation/epimerization NRPS domains reside in cosmids 008CH and 008CK having accession numbers IDAC 190901-3 and IDAC 190901-1, respectively.

In another embodiment, the isolated polynucleotide encoding a dual condensation/epimerization NRPS domain does not reside in the biosynthetic locus for syringomycin from Pseudomonas syringae pv. syringae strain B301D, the biosynthetic locus for syringopeptin from Pseudomonas syringae pv. syringae strain B301D, or the biosynthetic locus for a peptide natural product from Ralstonia solanacearum GMI 1000.

The invention also provides an isolated dual condensation/epimerization NRPS domain comprising at least 45% sequence identity to SEQ ID NO: 139. Certain embodiments expressly exclude one or more sequences, in particular the polypeptide sequence corresponding to the C-domains of NRPS protein of GenBank accession no. AAC80285 (SEQ ID NOS: 17, 19, 21 and 23); the polypeptide sequence corresponding to the C-domains of NRPS protein of GenBank accession no. AAF99707, AAO72424 and AAO72425 (SEQ ID NOS: 25, 27, 29, 31, 33, 35, 37, 39, 41, 43, 45, 47, 49, 51, 53, 55, 57 and 59) and the polypeptide sequence corresponding to the C-domains of NRPS protein of GenBank accession no. CAD17793 (Seq ID NOS: 61 and 63). Other sequences can be excluded without departing from the scope of the invention.

In a related aspect the invention provides an isolated dual condensation/epimerization NRPS domain comprising a polypeptide sequence selected from the group consisting of: (a) a sequence selected from the group consisting of SEQ ID NOS: 1, 3, 5, 7,9, 11, 13, 15, 17, 19, 21, 23, 25, 27, 29, 31, 33, 35, 37, 39, 41, 43, 45, 47, 49, 51, 53, 55, 57, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135 and 137; and (b) a sequence which has at least 60% or higher sequence similarity to said sequence of (a) as determined using the BLASTP 2.2.5 algorithm. Certain embodiments expressly exclude one or more sequences, in particular the polypeptide sequence corresponding to the C-domains of NRPS protein of GenBank accession no. AAC80285 (SEQ ID NOS: 17, 19, 21 and 23); the polypeptide sequence corresponding to the C-domains of NRPS protein of GenBank accession no. AAF99707, AAO72424 and AAO72425 (SEQ ID NOS: 25, 27, 29, 31, 33, 35, 37, 39, 41, 43, 45, 47, 49, 51, 53, 55, 57 and 59) and the polypeptide sequence corresponding to the C-domains of NRPS protein of GenBank accession no. CAD17793 (Seq ID NOS: 61 and 63).

In another related aspect the invention provides an isolated dual condensation/epimerization NRPS domain comprising a polypeptide sequence selected from the group consisting of: (a) a sequence selected from the group consisting of SEQ ID NOS: 1, 3, 5, 7,9, 11, 13, 15, 17, 19, 21, 23, 25, 27, 29, 31, 33, 35, 37, 39, 41, 43, 45, 47, 49, 51, 53, 55, 57, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135 and 137; and (b) a sequence which has at least 70% or higher sequence similarity to said sequence of (a) as determined using the BLASTP 2.2.5 algorithm. Certain embodiments expressly exclude one or more sequences, in particular the polypeptide sequence corresponding to the C-domains of NRPS protein of GenBank accession no. AAC80285 (SEQ ID NOS: 17, 19, 21 and 23); the polypeptide sequence corresponding to the C-domains of NRPS protein of GenBank accession no. AAF99707, AAO72424 and AAO72425 (SEQ ID NOS: 25, 27, 29, 31, 33, 35, 37, 39, 41, 43, 45, 47, 49, 51, 53, 55, 57 and 59) and the polypeptide sequence corresponding to the C-domains of NRPS protein of GenBank accession no. CAD17793 (Seq ID NOS: 61 and 63).

In one embodiment, the isolated dual condensation/epimerization NRPS domain is derived from an organism of the actinomycetes taxon. In another embodiment, the dual condensation/epimerization NRPS domain is not derived from the biosynthetic locus for syringomycin from Pseudomonas syringae pv. syringae strain B301D, the biosynthetic locus for syringopeptin from Pseudomonas syringae pv. syringae strain B301D and the biosynthetic locus for a peptide natural product from Ralstonia solanacearum GMI1000.

The present invention further contemplates several uses or applications of the dual condensation/epimerization NRPS domain isolated polynucleotide encoded by the isolated polynucleotide described herein. In one such use or application, the dual condensation/epimerization NRPS domain is involved in the incorporation of a D-amino acid or non-chiral amino acid into a peptide product. Other uses include: use of the dual condensation/epimerization NRPS domain for epimerization and condensation of amino acids in E-less NRPS systems; use of the dual condensation/epimerization NRPS domain to modify the stereochemistry of a synthesized peptide compound in vivo, using an appropriate recombinant host; use of the dual condensation/epimerization NRPS domain to modify the stereochemistry of a synthesized peptide compound in vitro, using purified enzymes supplemented with appropriate substrates; use of the dual condensation/epimerization NRPS domain to modify the strereochemistry of ramoplanin; and use of the dual condensation/epimerization NRPS domain to modify the stereochemistry of the complestatin molecule at a specific amino acid component. In one application, the dual condensation/epimerization NRPS domain is genetically substituted for a regular condensation domain in an expression system.

The present invention also encompasses expression vectors and cultured cells comprising the isolated polynucleotide encoding the dual condensation/epimerization NRPS domain. It also includes a method of producing the dual condensation/epimerization NRPS domain, the method comprising culturing the cells under conditions permitting expression of the dual condensation/epimerization NRPS domain and purifying the dual condensation/epimerization NRPS domain from the cell or the medium of the cell.

### BRIEF DESCRIPTION OF THE DRAWINGS

FIGS. 1a, 1b, 1c, 1d, 1e, 1f, 1g, 1h and 1i each represents a schematic view of the domain organization of NRPS systems. FIG. 1a shows epimerization domain-containing NRPS systems from Bacillus lichenformis ATCC 10716 (BACI) and Bacillus subtilis strain 168 (SURF); FIG. 1b shows epimerization domain-containing NRPS systems from Bacillus subtilis b213 (PLIP) and Streptomyces coelicolor A3(2) (CADA); FIG. 1c shows E-less NRPS systems from Actinoplanes sp. ATCC 33076 (RAMO) and Pseudomonas syringae pv. syringae strain B301D (SYRI); FIG. 1d shows E-less NRPS systems from Pseudomonas syringae pv. syringae strain B301D (SYRP) and Ralstonia solanacearum GMI1000; FIG. 1e shows E-less NRPS system from Streptomyces aizunensis NRRL B-11277 (023C); FIG. 1f shows E-less NRPS systems from Streptomyces griseofuscus NRRL B-5429 (034F) and Kitasatosporia sp. ECO-03 (040G); FIG. 1g shows E-less NRPS systems from Streptomyces sp. ECO-38 (084B) and Streptomyces sp. ECO-59 (107A); FIG. 1h shows E-less NRPS systems from Streptomyces viridifaciens NRRL ISP-5239 (143F) and Actinomadura sp. ATCC 39334 (153A); FIG. 1i shows E-less NRPS system from Micromonospora chersina ATCC 53710 (263B). Dual condensation/epimerization domains in FIGS. 1c to 1i are shown by a shaded circle.

FIG. 2 is a dendrogram showing the evolutionary relatedness of C-domains from various NRPS systems with a clearly branching cluster of representative condensation/epimerization NRPS domains of the invention (designated by a shaded circle) involved in the incorporation of D-amino acids into peptide products synthesized by E-less NRPS systems. Condensation domains located downstream of canonical epimerization domains are designated by black circles. Condensation domains involved in N-acylation of lipopeptides are designated by a clear circle. Regular C-domains that catalyze condensation of amino acids in the L- configuration are designated by hatched circles.

FIG. 3 represents the ramoplanin NRPS system present in Actinoplanes sp. ATCC 33076. The NRPS complex is composed of three polypeptides, 008CHP_{—}09, 008CHP_{—}10 and 008CHP_{—}11, composed of tripartite modules (C-A-T). Representative condensation/epimerization NRPS domains of the invention are represented by a shaded circle. 008CH and 008CK are deposited cosmids containing genes of the invention.

FIG. 4 illustrates the use of NRPS biosynthetic machinery to modify the stereochemistry of a synthesized peptide compound. Replacement of a regular condensation domain of the ramoplanin NRPS system (shown as a hatched circle) with a dual condensation/epimerization NRPS domain (shown as a shaded circle) alters the stereochemistry of ramoplanin at two different amino acid sites.

FIG. 5 illustrates the use of NRPS biosynthetic machinery to modify the stereochemistry of a synthesized peptide compound wherein a dual condensation/epimerization NRPS domain from the ramoplanin NRPS system is used to modify the stereochemistry of the complestatin molecule at a specific amino acid component.

### DETAILED DESCRIPTION

Dual condensation/Epimerization NRPS domains from NRPS systems in a variety of organisms were discovered and analyzed. For convenience, peptide biosynthetic loci and organisms containing NRPS systems with representative dual condensation/epimerization NRPS domains of the invention are referred to herein and in priority application U.S. Ser. No. 06/372,790 by reference to a source designation wherein “BACI” refers to the biosynthetic locus for bacitracin from Bacillus lichenformis ATCC 10716 (Konz et al. (1997), Chem. Biol., 4, 927-937); “SURF” refers to the biosynthetic locus for surfactin from Bacillus subtilis strain 168 (Cosmina et al. (1993), Mol. Microbiol., 8, 821-831); “PLIP” refers to the locus for plipastatin from Bacillus subtilis b213 (Steller et al. (1998), Chem. Biol., 6, 31-41); “CADA” refers to the calcium-dependent antibiotic from Streptomyces coelicolor A3(2) (Chong et al. (1998), Microbiology, 144, 193-199); “RAMO” refers to the biosynthetic locus for ramoplanin from Actinoplanes sp. ATCC 33076; “SYRI” refers to the biosynthetic locus for syringomycin from Pseudomonas syringae pv. syringae strain B301D (Guenzi et al. (1998), J. Biol. Chem., 273, 32857-32683) “SYRP” refers to the biosynthetic locus for syringopeptin from Pseudomonas syringae pv. syringae strain B301D (Scholz-Schroeder et aL. (2003), Mol. Plant Microbe Interact., 16, 271-280); URSO refers to the biosynthetic locus for a peptide from Ralstonia solanacearum (Salanoubat et al. (2002), Nature, 415, 497-502); 023C refers to the biosynthetic locus for a peptide from Streptomyces aizunensis NRRL B-11277; 034F refers to the biosynthetic locus for a peptide from Streptomyces griseofuscus NRRL B-5429; 040G refers to the biosynthetic locus for a peptide from Kitasatosporia sp. ECO-03; 084B refers to the biosynthetic locus for a peptide from Streptomyces sp. ECO-38; 107A refers to the biosynthetic locus for a peptide from Streptomyces sp. ECO-59; 143F refers to the biosynthetic locus for a peptide from Streptomyces viridifaciens NRRL ISP-5239; 153A refers to the biosynthetic locus for a peptide from Actinomadura sp. ATCC 39334, and 263B refers to the biosynthetic locus for a peptide from Micromonospora chersina ATCC 53710. All ECO numbers refer to organisms present in Ecopia's private culture collection.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, N.Y.).

As used herein, the terms “polynucleotide” or “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into anyone of numerous established kit formats which are well known in the art.

A “heterologous” (e.g. a heterologous gene) region of a DNA molecule is a subsegment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography . . . ). In addition, the protein of interest can be purified via affinity chromatography using polyclonal or monoclonal antibodies. The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgarno sequences, which serve as ribosome binding sequences during translation initiation. Promoters suitable for expressing the polypeptide or fragment thereof in bacteria include the E.coli lac or trp promoters, the lad promoter, the lacZ promoter, the T3 promoter, the T7 promoter, the gpt promoter, the lambda P_{R} promoter, the lambda P_{L} promoter, promoters from operons encoding glycolytic enzymes such as 3-phosphoglycerate kinase (PGK), and the acid phosphatase promoter. The vector may also include appropriate sequences for modulating expression levels, an origin of replication and a selectable marker.

As used herein, the designation “functional derivative” denotes, in the contexof a functional derivative of a sequence whether a nucleic acid or amino acid sequence, a molecule that retains a dual condensation/epimerization activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the dual condensation/epimerization activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecul which is substantially similar in structure and dual condensation/epimerization activity to the protein or nucleic acid of the present invention.

The functional derivatives of the present invention can be synthesized chemically or produced through recombinant DNA technology. All these methods are well known in the art.

As used herein, the term “purified” refers to a molecule having been separated from a cellular component. Thus, for example, a “purified protein” has been purified to a level not found in nature. A “substantially pure” molecule is a molecule that is lacking in most other cellular components.

For certainty, the sequences and polypeptides useful to practice the invention include without being limited thereto mutants, homologs, subtypes, alleles and the like. It shall be understood that generally, the sequences of the present invention should encode a functional (albeit defective) interaction domain. It will be clear to the person of ordinary skill that whether an interaction domain of the present invention, variant, derivative, or fragment thereof retains its function in binding to its partner can be readily determined by using the teachings and assays of the present invention and the general teachings of the art.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). The use of a mammalian cell as indicator can provide the advantage of furnishing an intermediate factor, which permits for example the interaction of two polypeptides which are tested, that might not be present in lower eukaryotes or prokaryotes. Of course, such an advantage might be rendered moot if both polypeptide tested directly interact. It will be understood that extracts from mammalian cells for example could be used in certain embodiments, to compensate for the lack of certain factors.

The expression a “dual condensation/epimerization NRPS domain” of the present invention is defined structurally as a polypeptide sequence that produces an alignment with at least 45% identity with the following consensus sequence (SEQ ID NO: 139) using the BLASTP 2.2.5 algorithm, with the filter option -F set to false, the gap opening penalty -G set to 11, the gap extension penalty -E set to 1, and all remaining options set to default values:

ADIYPLAPLQEGILFHHLIadggedDaYVIpavIeFDSReRLdaFIgALQ qViDRHDlLRTavvWeGLrEPVQVVwRhAeLpVeevtLdpagiaadpvaq LdaaaglrmDLgrAPLlrlhvAadpgggrWLaLLrfHHLVqDHTALevLI aEiqAfLaGrgdeLPePIPFRnFVAQARIGvsraEHErFFaeLLGDVtEP TAPFGLIDVrGDGsgveearlpldaeLaaRLReqARrLGVSpATlfHLAW ARVLgavSGRdDWFGTVLfGRMqaGaGADRvpGIFINTLPVRVrIggqg VldAVramRaqLAeLLeHEHAPLALAQRASGVpaptPLFTsLLNYRHsav aavsaealaawagaeleGirlLssrERTNYPLtVsVDDIGdgFsLtVqAv apiDaerVcallhTAlenLVdALEqaPdtpLsavdVLpaaERrrlLveWN dtaadyvpaatvpeLFeAQVartP

where consensus sequence SEQ ID NO: 139 is based on the sequences of representative dual condensation/epimerization NRPS domains from the following E-less NRPS-containing peptide biosynthetic loci: RAMO (SEQ ID NOS: 01, 03, 05, 07, 09, 11, 13 and 15); SYRI (SEQ ID NOS: 17, 19, 21 and 23); SYRP (SEQ ID NOS: 25, 27, 29, 31, 33, 35, 37, 39, 41, 43, 45, 47, 49, 51, 53, 55, 57 and 59); URSO (SEQ ID NOS: 61 and 63); 023C (SEQ ID NOS: 65, 67, 69, 71, 73, 75, 77, 79, 81, 83, 85, 87 and 89); 034F (SEQ ID NOS: 91, 93, 95 and 97); 040G (SEQ ID NOS: 99 and 101); 084B (SEQ ID NOS: 103,105, 107 and 109); 107A (SEQ ID NOS: 111 and 113); 143F (SEQ ID NOS: 115, 117, 119 and 121); 153A (SEQ ID NOS: 123, 125, 127 and 129); and 263B (SEQ ID NOS: 131, 133, 135 and 137).

The consensus sequence was generated as follows. First, the listed sequences were aligned with the ClustaIX 1.81 program using default settings. Then a profile hidden Markov model (HMM) was made from the alignment file with the hmmbuild program of the HMMER 2.2 package (Sean Eddy, Washington University; world-wide-web hmmer.wustl.edu/) and was calibrated with the hmmcalibrate program of the HMMER package, both using default settings. Briefly, a profile hidden Markov model is a statistical description of a sequence family's consensus. HMMER is a freely distributable implementation of profile HMM software for protein sequence analysis and is available from the above web site. Finally, the consensus sequences were generated from the HMM with the hmmemit program of the HMMER package using the -c option so as to predict a single majority rule consensus sequence from the HMM's probability distribution. Highly conserved amino acid residues (p>=0.5) are shown in upper case in the consensus sequence, others are shown in lower case.

The expression a “polynucleotide encoding a dual condensation/epimerization NRPS domain” refers to a polynucleotide encoding a dual condensation/epimerization NRPS domain. Representative examples of a polynucleotide encoding a dual condensation/epimerization NRPS domains of the invention include the polynucleotides encoding the dual condensation/epimerization NRPS domains residing in RAMO, i.e. SEQ ID NOS: 2, 4, 6, 8, 10,12, 14 and 16; SYRI, i.e. SEQ ID NOS: 18, 20, 22 and 24; SYRP, i.e. SEQ ID NOS: 26, 28, 30, 32, 34, 36, 38, 40, 42, 44, 46, 48, 50, 52, 54, 56, 58 and 60; URSO, i.e. SEQ ID NOS: 62 and 64; 023C, i.e. SEQ ID NOS: 66, 68, 70, 72, 74, 76, 78, 80, 82, 84, 86, 88 and 90; 034F, i.e. SEQ ID NOS: 92, 94, 96 and 98; 040G, i.e. SEQ ID NOS: 100 and 102); 084B, i.e. SEQ ID NOS: 104, 106, 108 and 110; 107A, i.e. SEQ ID NOS: 112 and 114; 143F, i.e. SEQ ID NOS: 116,118,120 and 122; 153A, i.e. SEQ ID NOS: 124, 126, 128 and 130; and 263B, i.e. SEQ ID NOS: 132, 134, 136 and 138.

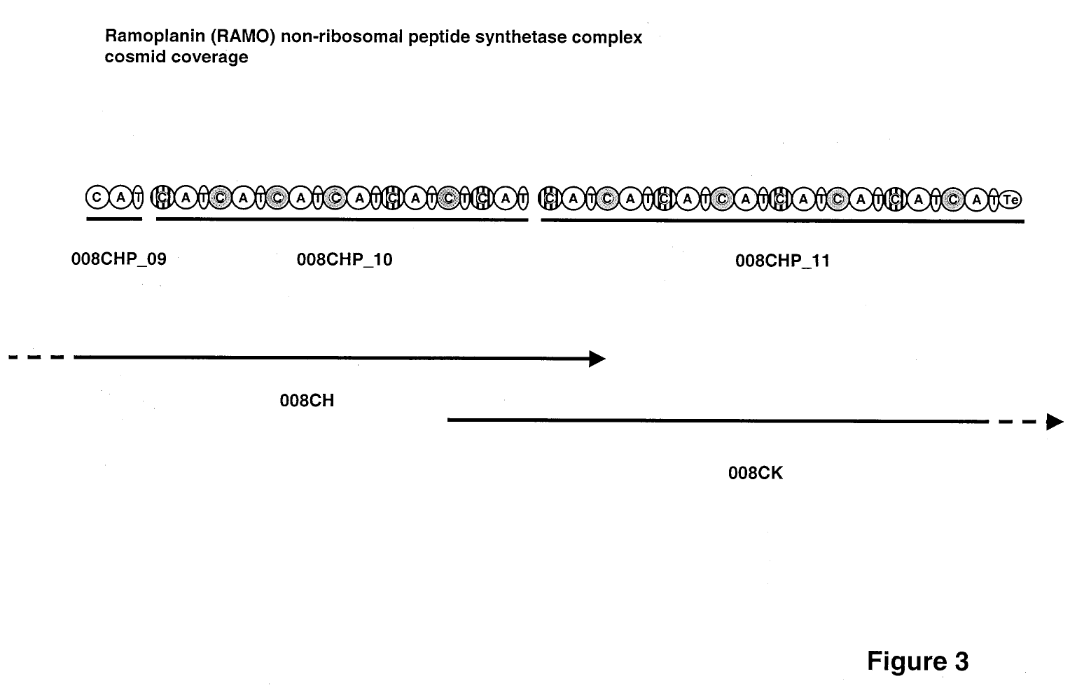

Cosmid clones containing genes and proteins of the invention have been deposited with the International Depositary Authority of Canada, Bureau of Microbiology, Health Canada, 1015 Arlington Street, Winnipeg, Manitoba, Canada R3E 3R2 under the terms of the Budapest Treaty on the International Recognition of the Deposit of Microorganisms for Purposes of Patent Procedure. An E. coli DH10B strain harboring cosmid clone 008CH containing a polynucleotide encoding dual condensation/epimerization NRPS domains SEQ ID NOS: 2, 4, 6 and 8 in the biosynthetic locus for ramoplanin from Actinoplanes sp. ATCC 33076 was deposited on Sep. 19, 2001 and assigned accession number IDAC 190901-3 (FIG. 3). An E. coli DH10B strain harboring cosmid clone 008CK condensation/epimerization dual domains with SEQ ID NOS: 10, 12, 14 and 16 in the biosynthetic locus for ramoplanin from Actinoplanes sp. ATCC 33076 was deposited on Sep. 19, 2001 and assigned accession number IDAC 190901-1 (FIG. 3). The E. coli strain deposits are referred to herein as “the deposited strains”. The sequences of the nucleotides encoding the dual condensation/epimerization NRPS domains of the invention present in the deposited strains as well as the amino acid sequences of the corresponding polypeptides are controlling in the event of any conflict with any description of sequences herein. A license may be required to make, use or sell the deposited strains, nucleic acids therein or compounds derived therefrom, and no such license is hereby granted.

The expression “a condition of high stringency” refers to any one of the hybridization conditions described herein, and includes other “high stringency” conditions known in the art. In one condition, a polymer membrane containing immobilized denatured nucleic acids is first prehybridized for 30 minutes at 45 ° C. in a solution consisting of 0.9 M NaCl, 50 mM NaH₂PO₄, pH 7.0, 5.0 mM Na₂EDTA, 0.5% SDS, 10×Denhardt's, and 0.5 mg/ml polyriboadenylic acid. Approximately 2×10⁷ cpm (specific activity 4-9×10⁸ cpm/ug) of ³²P end-labeled oligonucleotide probe are then added to the solution. After 12-16 hours of incubation, the membrane is washed for 30 minutes at room temperature in 1×SET (150 mM NaCl, 20 mM Tris hydrochloride, pH 7.8, 1 mM Na ₂EDTA) containing 0.5% SDS, followed by a 30 minute wash in fresh 1×SET at Tm-10° C. for the oligonucleotide probe, where Tm is the melting temperature of the probe. Stringency may be varied by conducting the hybridization at varying temperatures below the melting temperatures of the probes. The melting temperature of the probe may be calculated using the following formula: for oligonucleotide probes between 14 and 70 nucleotides in length, the melting temperature (Tm) in degrees Celcius may be calculated using the formula: Tm=81.5+16.6(log [Na⁺])+0.41(fraction G+C)−(600/N), where N is the length of the oligonucleotide. If the hybridization is carried out in a solution containing formamide, the melting temperature may be calculated using the equation Tm=81.5+16.6(log [Na⁺])+0.41(fraction G+C)−(0.63% formamide)−(600/N), where N is the length of the probe. For probes over 200 nucleotides in length, the hybridization may be carried out at 15-25° C. below the Tm. For shorter probes, such as oligonucleotide probes, the hybridization may be conducted at 5-10° C. below the Tm. Preferably, the hybridization is conducted in 6×SSC. for shorter probes and the hybridization is conducted in 50% formamide containing solutions for longer probes.

The term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters, which aligns the polynucleotides or fragments being compared and determines the extent of nucleotide identity between them. “Homology”, with respect to polypeptides (i.e., amino acids), may be determined using a program, such as BLASTP version 2.2.5 with the default parameters, which aligns the polypeptides or fragments being compared and determines the extent of amino acid identity or similarity between them. It will be appreciated that amino acid “homology” includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and lle with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue. A “homology of 70% or higher” includes a homology of, for example, 70%, 75%, 80%, 85%, 90%, 95%, and up to 100% (identical) between two or more nucleotide or amino acid sequences. A “homology of at least 45%” includes a homology of, for example, 45%, 50%, 60%, 70%, 80%, 90%, and up to 100% (identical) between two or more nucleotide or amino acid sequences.

We observed the surprising existence of NRPS systems lacking cononical epimerization domains but nonetheless appearing to direct the incorporation of D-amino acids (and occasionally non-chiral amino acids) into their resulting peptide products. We further noted several conserved motifs shared between C and E domains and investigated our hypothesis that E-less NRPSs systems may contain specialized dual function C domains that can epimerize the amino acid activated by the upstream module. We identified unusual C-domains downstream of the T domain carrying the amino acid destined to be epimerized. This position is where one would expect to find a canonical E domain. These unusual C-domains positioned downstream of modules that incorporate D-amino acids (and occasionally non-chiral amino acids) appeared to have dual condensation and epimerization acitivity and hence we refer to them as “dual condensation/epimerization NRPS domains”. These dual condensation/epimerization NRPS domains in E-less NRPSs systems allow for accurate predictions of the stereochemistry of the peptide product.

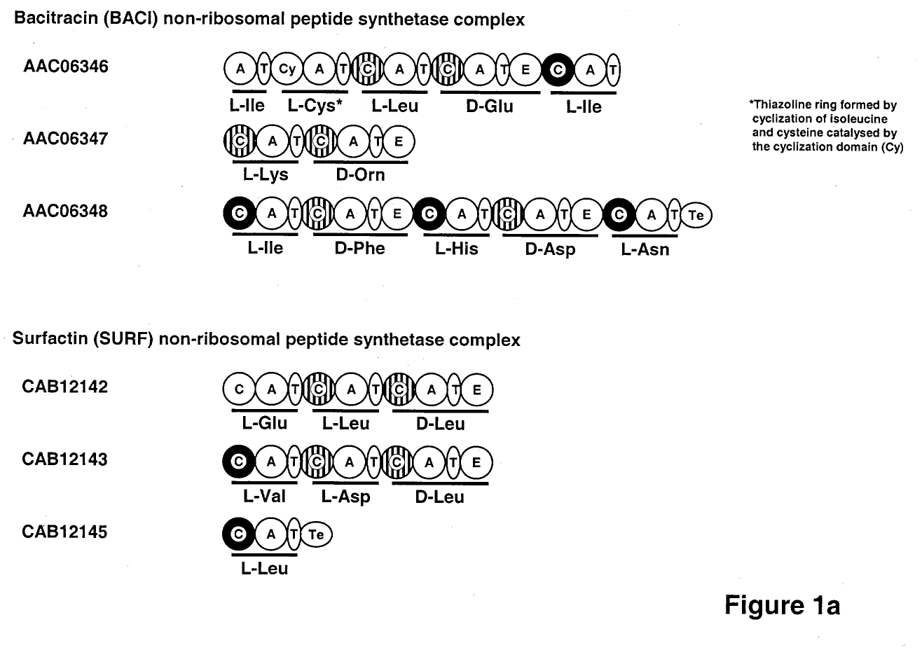

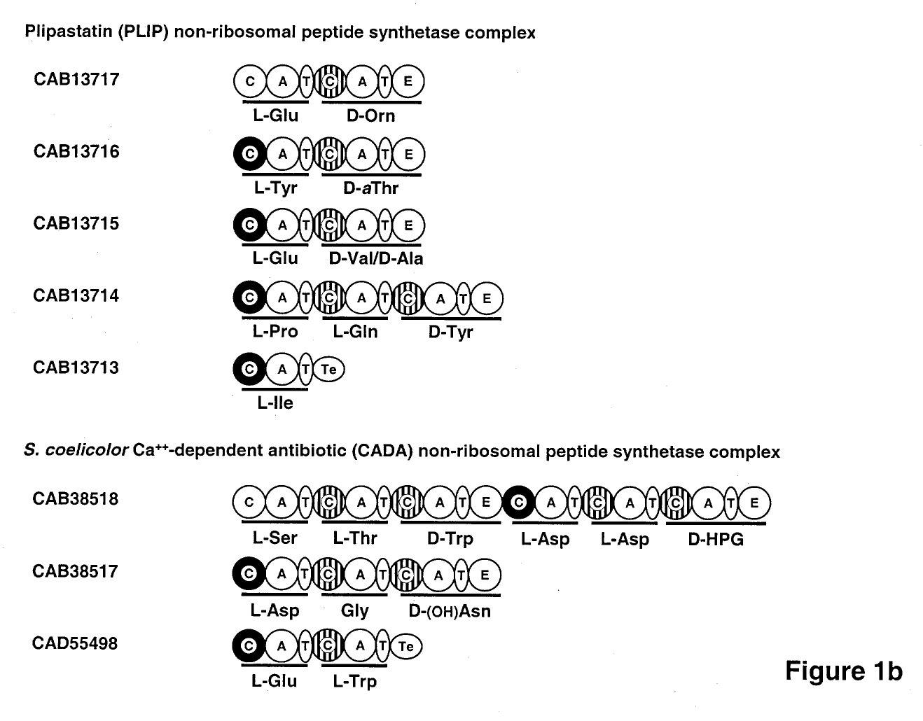

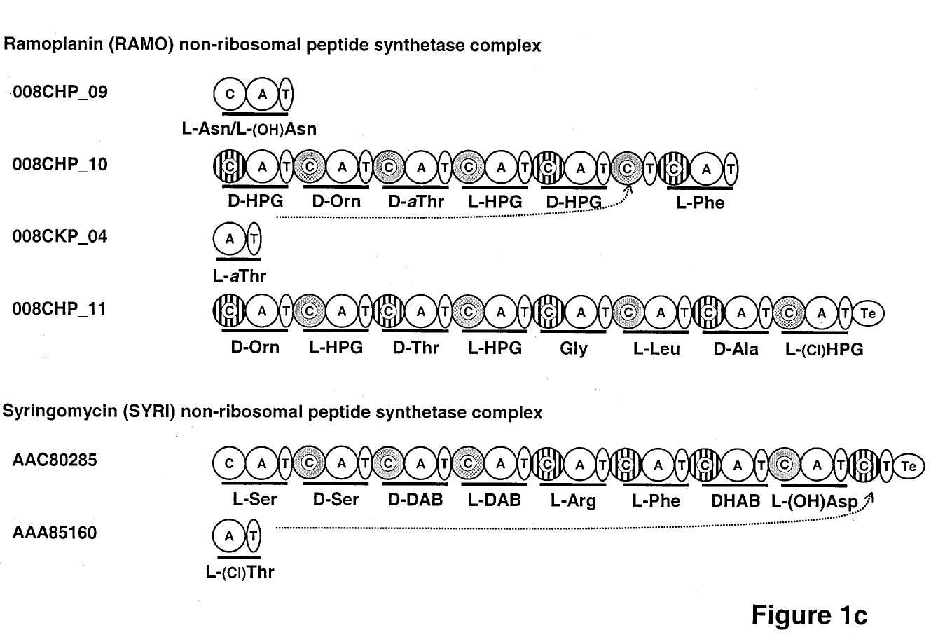

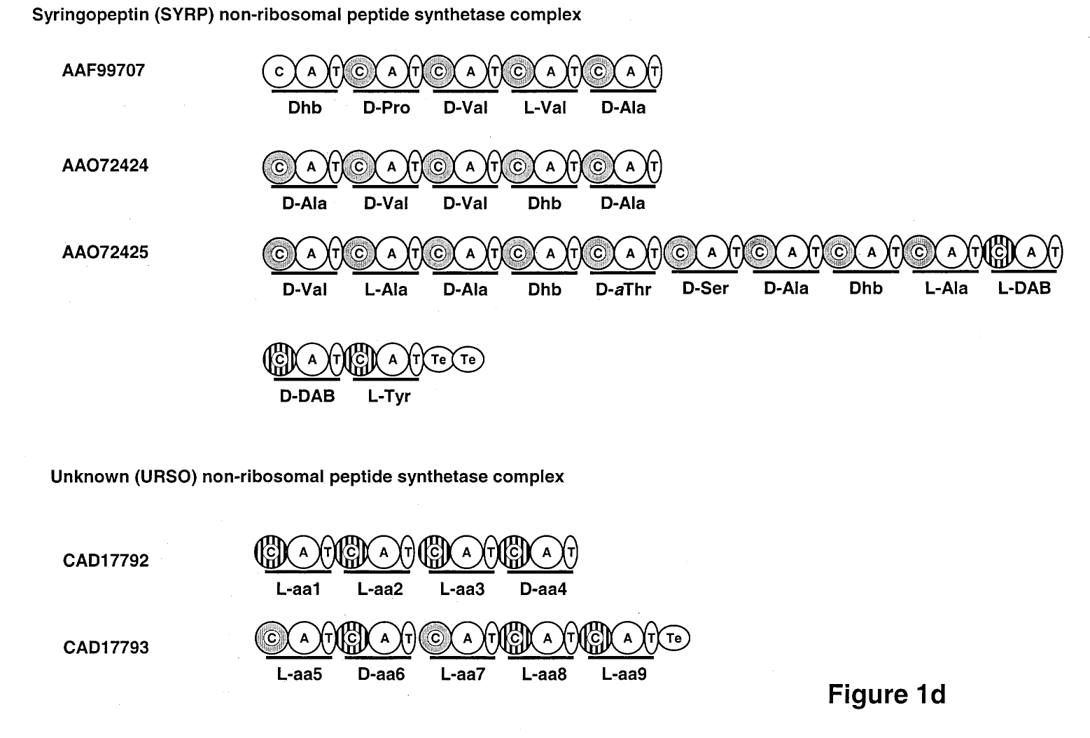

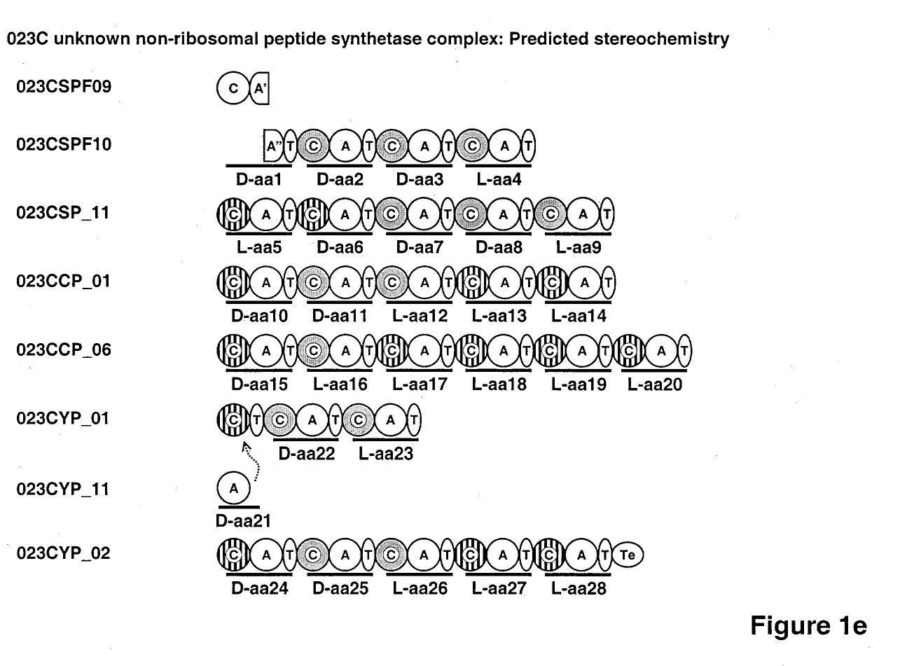

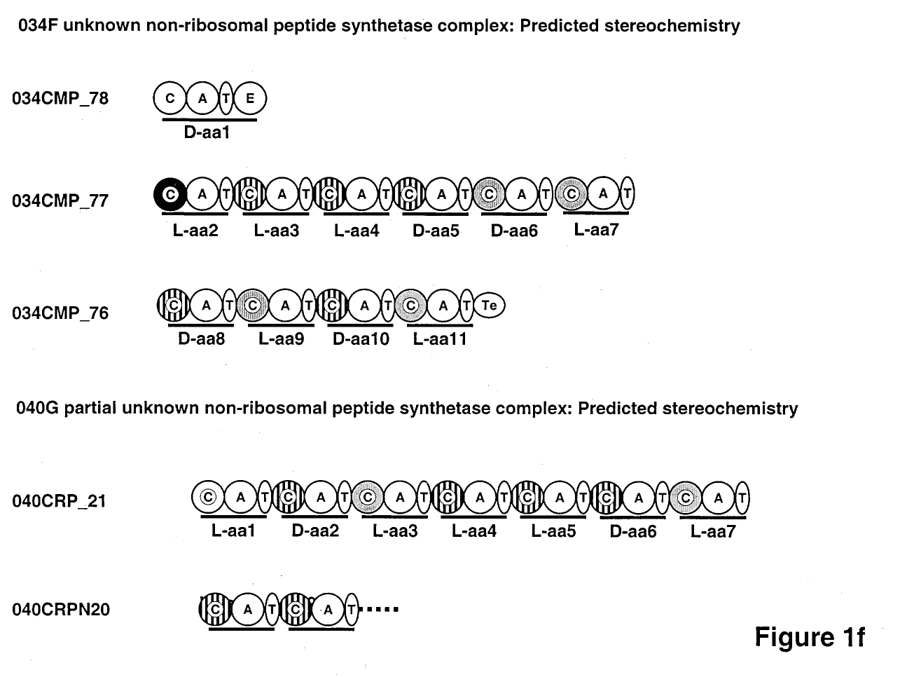

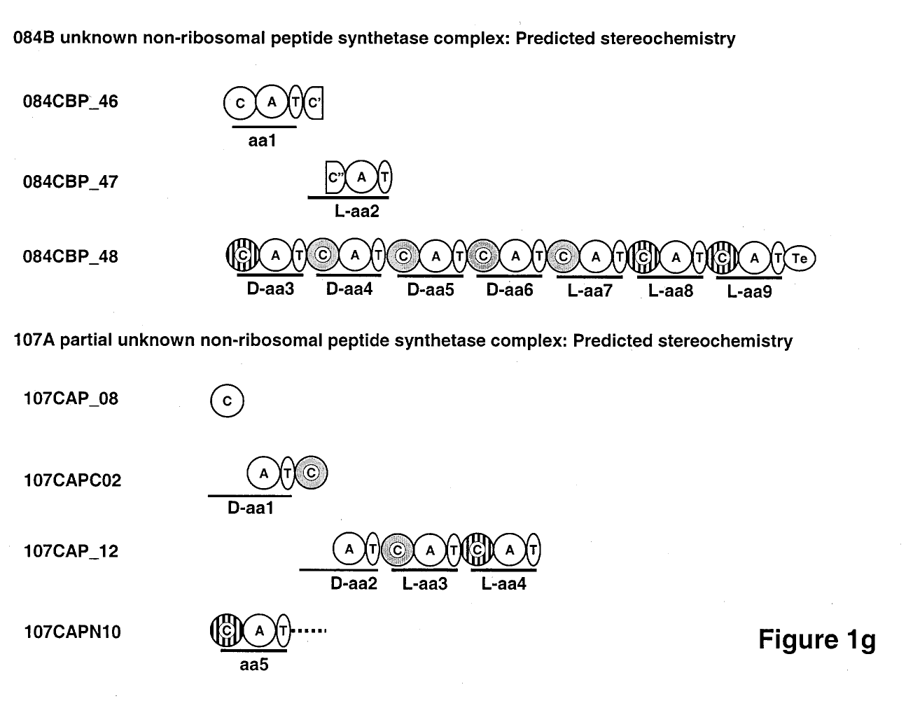

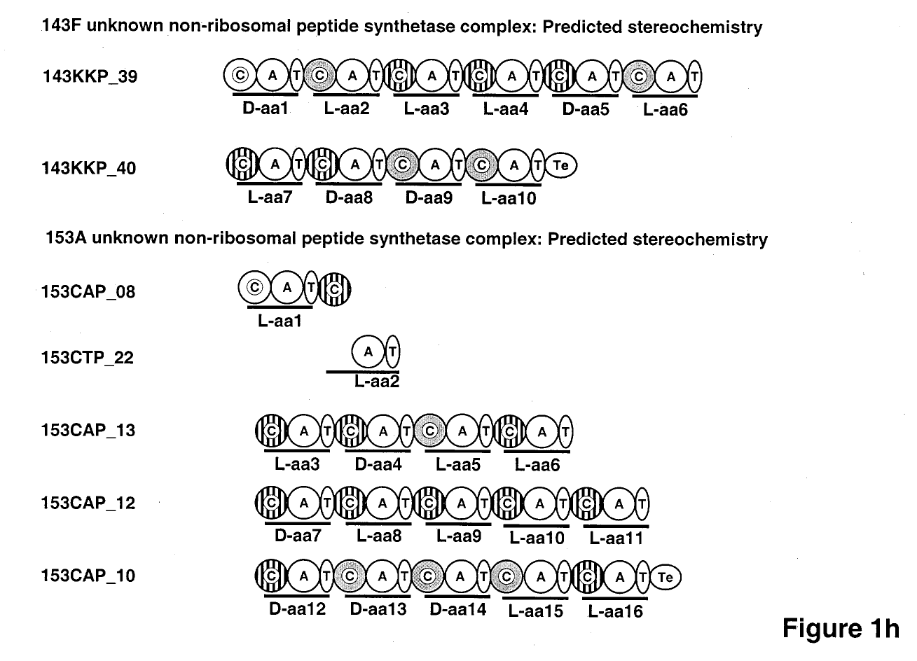

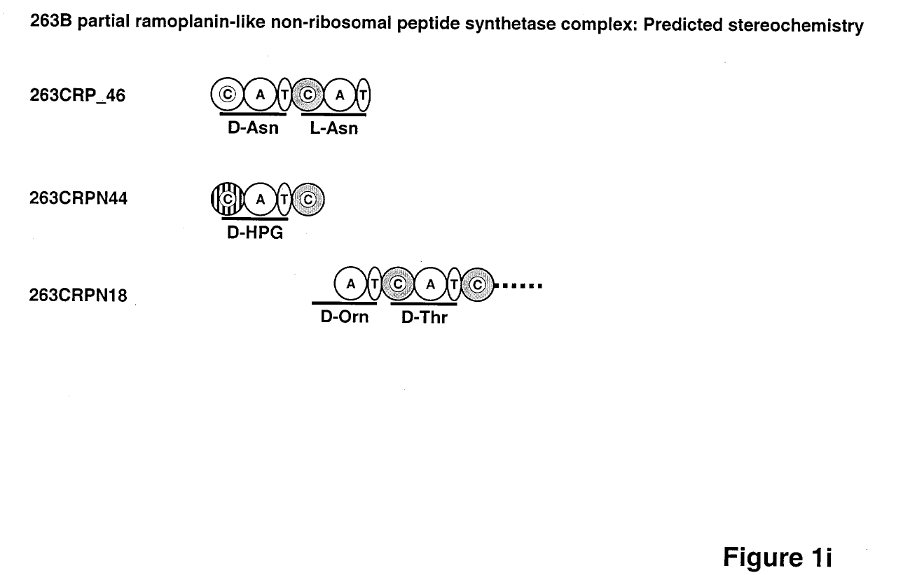

Condensation domains from the E-less peptide biosynthetic clusters RAMO, SYRI, SYRP, URSO, 023C, 034F, 034G, 084B, 107A, 143F, 153A, and 263B were identified and analyzed. FIG. 1 lists the NRPS synthetase complexes from which condensation domains were compared. NRPS from which condensation domains were analyzed were as follows: BACI open reading frames (ORFs) with GenBank accession numbers AAC06346 to AAC06348; SURF ORFs with GenBank accession numbers CAB12142, CAB12143 and CAB12145; from PLIP ORFs with GenBank accession numbers CAB13713 to CAB13717; from CADA ORFs with GenBank accession numbers CAB38517, CAB38518 and CAD55498; from RAMO ORFs with Ecopia accession numbers 008CHP_{—}09, 008CHP_{—}10 and 008CHP_{—}11 (ORFs 12, 13 and 14, respectively, as defined in U.S. application U.S. Ser. No. 09/976,059); from SYRI ORFs with GenBank accession numbers AAC80285 and AAA85160; from SYRP ORFs with GenBank accession numbers AAF99707, AAO72424 and AAO72425; from URSO ORFs with GenBank accession numbers CAD17792 and CAD17793; 023C. ORFs with Ecopia accession numbers 023CSPF09, 023CSPF10, 023CSP_{—}11, 023CCP_{—}01, 023CCP_{—}06, 023CYP_{—}01 and 023CYP_{—}02; 034F ORFs with Ecopia accession numbers 034CMP_{—}76 to _{—}78; 040G ORFs with Ecopia accession numbers 04ORP_{—}21 and 040CRPN20; 084B ORFs with Ecopia accession numbers 084CBP_{—}46 to 48; from 107A ORFs with Ecopia accession numbers 107CAP08, 107CAPC02, 107CAP_{—}12 and 107CAPN10; 143F ORFs with Ecopia accession numbers 143KKP_{—}39 and 143KK_{—}40; 153A ORFs with Ecopia accession numbers 153CAP_{—}08, 153CAP_{—}13, 153CAP_{—}12 and 153CAP_{—}10 and 263B″ ORFs with Ecopia accession numbers 263CRP_{—}46, 263CRPN44 and 263CRPN18.

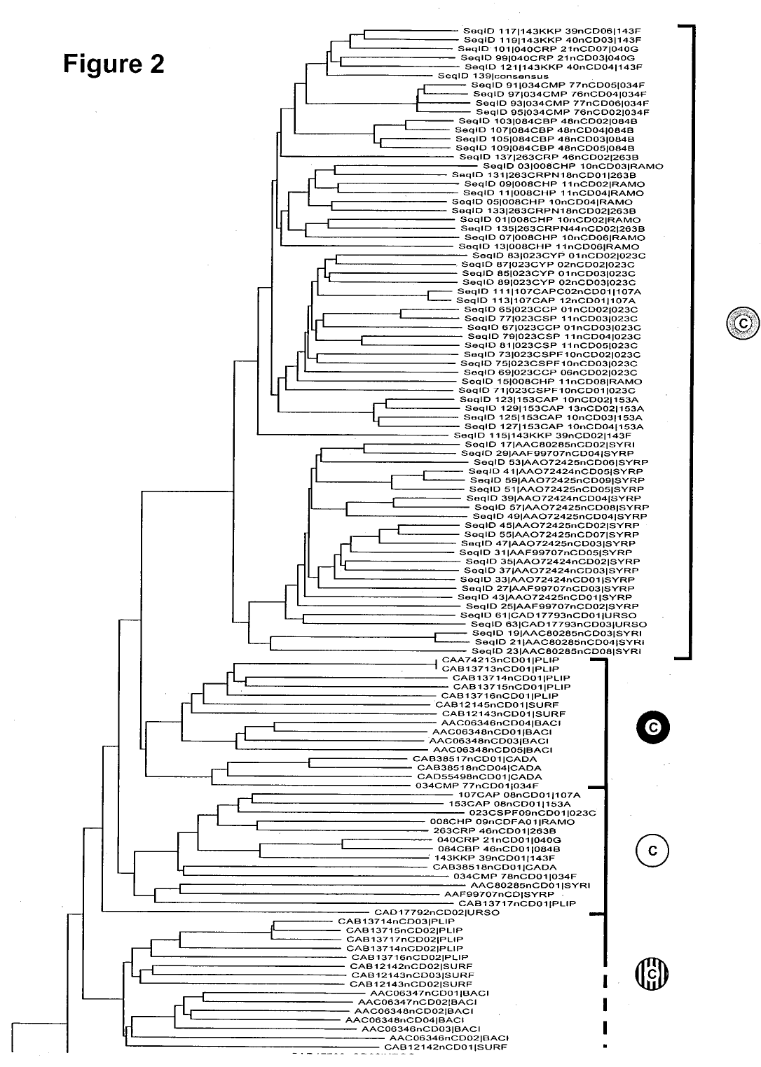

The amino acid sequences of the condensation domains from the NRPS systems listed in FIG. 1 were subjected to multiple sequence alignment using ClustalW ™ (Thompson et al (1994), Nucl. Acids Res., 22, 4673-4680). In FIG. 2, NRPS C domain sequences obtained from the GenBank database are denoted by accessions beginning with three letters and followed by digits (usually numbering 5). These first eight characters correspond to the GenBank accession number, followed by a lower case “n” denoting an NRPS domain, followed by the letters “CD” and two digits denoting “C domain” and its number relative to the other C domains contained on that polypeptide sequence. Thus, “AAC80285nCD02|SYRI” represents the amino acid sequence corresponding to the second C domain contained on the GenBank entry AAC80285 for an NRPS from the syringomycin biosynthetic locus. NRPS C domain sequences having Ecopia accession numbers follow the same nomenclature (nCD00) but are characterized by a root of nine-character accessions beginning with three numbers.

The condensation domains shown in FIG. 2 are divided in four classes each identified by circles. Empty circles depict condensation domains in the first module of lipopeptide NRPS systems which condensation domains are involved in the N-acyl capping mechanism described in co-pending application U.S. Ser. No. 10/329,027. The teachings of U.S. Ser. No. 10/329,027 are incorporated herein by reference. Hatched circles depict condensation domains that follow modules that incorporate L- amino acids. C domains that cluster above the N-acyl capping C domains in FIG. 2 (empty circles) carry out condensation reaction between a D- form amino acid (or occasionally a non-chiral amino acid) activated by the upstream module and the amino acid activated by the cognate module. These “D-specific” C domains can be divided into two categories; one category of “D-specific” C domains (black circles) includes those that follow C-A-T-E modules and the other category of “D-specific” C domains (shaded circles) includes specialized C domains that follow C-A-T modules that incorporate D- amino acids (or non-chiral amino acids). Phylogenetic analysis of the amino acid sequences of these specialized C domains suggests, at the very least, that they condense the D-form of the upstream amino acid to the cognate amino acid as they surprisingly cluster together with C domains that follow C-A-T-E modules that incorporate D- amino acids in E-containing NRPS systems (FIG. 2). Therefore epimerization of amino acid residues by E-less NRPSs is likely to occur post activation but prior to condensation. These specialized C domains from E-less NRPSs, referred to herein as dual condensation/epimerization NRPS domains direct the epimerization of the amino acid activated by the upstream module.

Without being limited to any particular mechanism, the dual condensation/epimerization activity may involve the recruitment of a cellular enzyme that provides the epimerization activity in trans akin to the transacting adenylation domain in the syringomycin and ramoplanin NRPS system. In this scenario, one would predict that a highly purified syringomycin or ramoplanin NRPS complex would be incapable of generating products with D-amino acids without addition of a cellular extract containing the trans-acting epimerization activity. Alternatively, the dual condensation/epimerization NRPS domains may directly catalyze the epimerization of the amino acid activated by the upstream module prior to its condensation with the amino acid activated by the cognate module. In this scenario, the dual condensation/epimerization NRPS domains would have the inherent ability to carry out both a condensation reaction as well as an epimerization reaction. Such dual function has been found in other domains, for example, “cyclization” domains (also sometimes referred to as heterocyclization domains) which are capable of carrying out both a condensation reaction and a cyclization reaction (Doekel and Marahiel, supra).

The dual condensation/epimerization NRPS domains of the invention are expected to be found in a variety of E-less NRPS systems producing peptide products containing D- or non-chiral amino acids. NRPS systems may be found in a variety of microorganisms. Preferred microorganisms expected to contain the dual condensation/epimerization NRPS domains include but are not limited to bacteria of the order Actinomycetales, also referred to as actinomycetes. Preferred genera of Actinomycetes include Nocardia, Geodermatophilus, Actinoplanes, Micromonospora, Nocardioides, Saccharothrix, Amycolatopsis, Kutzneria, Saccharomonospora, Saccharopolyspora, Kitasatospora, Streptomyces, Microbispora, Streptosporangium, Actinomadura. The taxonomy of actinomycetes is complex and reference is made to Goodfellow (1989) Suprageneric classification of actinomycetes, Bergey's Manual of Systematic Bacteriology, Vol. 4, Williams and Wilkins, Baltimore, pp 2322-2339, and to Embley and Stackebrandt, (1994). The molecular phylogeny and systematics of the actinomycetes, Annu. Rev. Microbiol. 48, 257-289, for genera that may also contain dual condensation/epimerization NRPS domains of the present invention. One skilled in the art would understand that a NRSP complex that lacks canonical epimerization domains but encodes a peptide containing amino acid residue(s) in the D-Stereochemistry would likely be a candidate to contain dual condensation/epimerization NRPS domains of the present invention.

Many of the E-less NRPSs with dual condensation/epimerization NRPS domains produce lipopeptides and many of these E-less NRPSs include a trans-acting A domain. They have been identified in very diverse microbes including three genera belonging to the high GC gram positive bacteria commonly known as actinomycetes, namely Actinoplanes, Actinomadura, Micromonospora and Streptomyces, as well as the gram negative bacterium Pseudomonas syringae pv. syringae and the proteobacterium Ralstonia solanacearum. E-less NRPSs with the dual epimerization/condensation NRPS domains are expected to be widespread in nature.

The dual condensation/epimerization NRPS domains of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137 and 139 were compared using the BLASTP algorithm with the default parameters to the sequences of the National Center for Biotechnology Information (NCBI) nonredundant protein database and to sequences of the DECIPHER®) database of microbial genes, pathways and natural products (Ecopia BioSciences Inc., St-Laurent, Canada). The accession numbers of the top GenBank hits of this BLAST analysis are presented in Table 1 along with the corresponding E values. The E value assists in the determination of whether two sequences display sufficient similarity to justify an inference of homology. The E value relates the expected number of chance alignments with an alignment score at least equal to the observed alignment score. An E value of 0.00 indicates a perfect homolog. The E-values are calculated as described in Altschul et al. (1990), J. MoL Biol. 215, 403-410; Gish et al. (1993), Nature Genetics 3, 266-272.

| | | | | | | | | proposed function of |
|---|---|---|---|---|---|---|---|---|
| SeqID | ORF accession | Locus | #aa | GenBank homology | probability | % identity | % similarity | GenBank match |
| 1 | 008CHP_10nCD02 | RAMO | 456 | AAF99707.2,5379aa | 1e − 100 | 204/470 (43.4%) | 282/470 (60%) | syringopeptin synthetase, |
| | | | | | | | | Pseudomonas syringae |
| | | | | | | | | pv.syringae |
| | | | | ZP_00126388.1,3699aa | 1e − 98 | 214/479 (44.68%) | 286/479 (59.71%) | hypothetical protein, |
| | | | | | | | | Pseudomonas syringae |
| | | | | | | | | pv.syringae B728a |
| | | | | AAO72424.1,5457aa | 2e − 98 | 204/468 (43.59%) | 282/468 (60.26%) | syringopeptin |
| | | | | | | | | synthetase B, |
| | | | | | | | | Pseudomonas syringae |
| | | | | | | | | pv.syringae |
| 3 | 008CHP_10nCD03 | RAMO | 451 | AAF99707.2,5379aa | 4e − 93 | 201/467 (43.04%) | 271/467 (58.03%) | syringopeptin synthetase, |
| | | | | | | | | Pseudomonas syringae |
| | | | | | | | | pv.syringae |
| | | | | AAO72425.1,13536aa | 2e − 89 | 198/469 (42.22%) | 265/469 (56.5%) | syringopeptin |
| | | | | | | | | synthetase C, |
| | | | | | | | | Pseudomonas syringae |
| | | | | | | | | pv.syringae |
| | | | | AAO72424.1,5457aa | 5e − 89 | 188/447 (42.06%) | 259/447 (57.94%) | syringopeptin |
| | | | | | | | | synthetase B, |
| | | | | | | | | Pseudomonas syringae |
| | | | | | | | | pv.syringae |
| 5 | 008CHP_10nCD04 | RAMO | 461 | AAO72425.1,13536aa | 8e − 98 | 207/470 (44.04%) | 271/470 (57.66%) | syringopeptin |
| | | | | | | | | synthetase C, |
| | | | | | | | | Pseudomonas syringae |
| | | | | | | | | pv.syringae |
| | | | | ZP_00126883.1,2901aa | 1e − 97 | 207/470 (44.04%) | 270/470 (57.45%) | hypothetical protein, |
| | | | | | | | | Pseudomonas syringae |
| | | | | | | | | pv.syringae B728a |
| | | | | BAC67536.1,5924aa | 5e − 95 | 205/473 (43.34%) | 267/473 (56.45%) | arthrofactin synthetase C, |
| | | | | | | | | Pseudomonas sp. MIS38 |
| 7 | 008CHP_10nCD06 | RAMO | 445 | ZP_00123731.1,5345aa | 5e − 83 | 178/390 (45.64%) | 233/390 (59.74%) | hypothetical protein, |
| | | | | | | | | Pseudomonas syringae |
| | | | | | | | | pv.syringae B728a |
| | | | | BAC67536.1,5924aa | 9e − 83 | 182/389 (46.79%) | 229/389 (58.87%) | arthrofactin synthetase C, |
| | | | | | | | | Pseudomonas sp. MIS38 |
| | | | | AAO72425.1,13536aa | 2e − 82 | 175/388 (45.1%) | 238/388 (61.34%) | syringopeptin |
| | | | | | | | | synthetase C, |
| | | | | | | | | Pseudomonas syringae |
| | | | | | | | | pv.syringae |
| 9 | 008CHP_11nCD02 | RAMO | 461 | AAF99707.2,5379aa | 5e − 99 | 201/452 (44.47%) | 284/452 (62.83%) | syringopeptin synthetase, |
| | | | | | | | | Pseudomonas syringae |
| | | | | | | | | pv.syringae |
| | | | | ZP_00126883.1,2901aa | 2e − 98 | 208/471 (44.16%) | 284/471 (60.3%) | hypothetical protein, |
| | | | | | | | | Pseudomonas syringae |
| | | | | | | | | pv.syringae B728a |
| | | | | AAO72425.1,13536aa | 3e − 98 | 208/471 (44.16%) | 283/471 (60.08%) | syringopeptin |
| | | | | | | | | synthetase C, |
| | | | | | | | | Pseudomonas syringae |
| | | | | | | | | pv.syringae |
| 11 | 008CHP_11nCD04 | RAMO | 462 | AAO72425.1,13536aa | 1e − 107 | 214/477 (44.86%) | 289/477 (60.59%) | syringopeptin |
| | | | | | | | | synthetase C, |
| | | | | | | | | Pseudomonas syringae |
| | | | | | | | | pv.syringae |
| | | | | ZP_00126883.1,2901aa | 1e − 107 | 214/477 (44.86%) | 288/477 (60.38%) | hypothetical protein, |
| | | | | | | | | Pseudomonas syringae |
| | | | | | | | | pv.syringae B728a |
| | | | | AAO72424.1,5457aa | 1e − 107 | 214/469 (45.63%) | 292/469 (62.26%) | syringopeptin |
| | | | | | | | | synthetase B, |
| | | | | | | | | Pseudomonas syringae |
| | | | | | | | | pv.syringae |
| 13 | 008CHP_11nCD06 | RAMO | 461 | AAO72425.1,13536aa | 2e − 95 | 203/449 (45.21%) | 255/449 (56.79%) | syringopeptin |
| | | | | | | | | synthetase C, |
| | | | | | | | | Pseudomonas syringae |
| | | | | | | | | pv.syringae |
| | | | | ZP_00126883.1,2901aa | 2e − 95 | 203/449 (45.21%) | 255/449 (56.79%) | hypothetical protein, |
| | | | | | | | | Pseudomonas syringae |
| | | | | | | | | pv.syringae B728a |
| | | | | NP_522203.1,5953aa | 3e − 90 | 203/453 (44.81%) | 262/453 (57.84%) | probable peptide |
| | | | | | | | | synthase, |
| | | | | | | | | Ralstonia solanacearum |
| 15 | 008CHP_11nCD08 | RAMO | 469 | ZP_00086025.1,5422aa | 1e − 93 | 198/474 (41.77%) | 266/474 (56.12%) | hypothetical protein, |
| | | | | | | | | Pseudomonas fluorescens |
| | | | | | | | | PfO-1 |
| | | | | AAO72425.1,13536aa | 1e − 92 | 198/474 (41.77%) | 270/474 (56.96%) | syringopeptin |
| | | | | | | | | synthetase C, |
| | | | | | | | | Pseudomonas syringae |
| | | | | | | | | pv.syringae |
| | | | | ZP_00126883.1,2901aa | 4e − 92 | 197/474 (41.56%) | 270/474 (56.96%) | hypothetical protein, |
| | | | | | | | | Pseudomonas syringae |
| | | | | | | | | pv.syringae B728a |
| 17 | AAC80285nCD02 | SYRI | 466 | T14593,9376aa | 0.0 | 466/466 (100%) | 466/466 (100%) | syringomycin synthetase, |
| | | | | | | | | Pseudomonas syringae |
| | | | | | | | | pv.syringae |
| | | | | ZP_00123737.1,9498aa | 0.0 | 442/468 (94.44%) | 446/468 (95.3%) | hypothetical protein, |
| | | | | | | | | Pseudomonas syringae |
| | | | | | | | | pv.syringae B728a |
| | | | | AAF99707.2,5379aa | 0.0 | 353/468 (75.43%) | 386/468 (82.48%) | syringopeptin synthetase, |
| | | | | | | | | Pseudomonas syringae |
| | | | | | | | | pv.syringae |
| 19 | AAC80285nCD03 | SYRI | 461 | T14593,9376aa | 0.0 | 437/461 (94.79%) | 437/461 (94.79%) | syringomycin synthetase, |
| | | | | | | | | Pseudomonas syringae |
| | | | | | | | | pv.syringae |
| | | | | ZP_00123737.1,9498aa | 0.0 | 433/461 (93.93%) | 434/461 (94.14%) | hypothetical protein, |
| | | | | | | | | Pseudomonas syringae |
| | | | | | | | | pv.syringae B728a |
| | | | | AAL57600.1,3316aa | 1e − 114 | 216/468 (46.15%) | 282/468 (60.26%) | peptide synthetase XpsB, |
| | | | | | | | | Xenorhabdus bovienii |
| 21 | AAC80285nCD04 | SYRI | 461 | T14593,9376aa | 0.0 | 438/461 (95.01%) | 438/461 (95.01%) | syringomycin synthetase, |
| | | | | | | | | Pseudomonas syringae |
| | | | | | | | | pv.syringae |
| | | | | ZP_00123737.1,9498aa | 0.0 | 437/461 (94.79%) | 438/461 (95.01%) | hypothetical protein, |
| | | | | | | | | Pseudomonas syringae |
| | | | | | | | | pv.syringae B728a |
| | | | | AAL57600.1,3316aa | 1e − 117 | 219/468 (46.79%) | 293/468 (62.61%) | peptide synthetase XpsB, |
| | | | | | | | | Xenorhabdus bovienii |
| 23 | AAC80285nCD08 | SYRI | 470 | T14593,9376aa | 0.0 | 470/470 (100%) | 470/470 (100%) | syringomycin synthetase, |
| | | | | | | | | Pseudomonas syringae |
| | | | | | | | | pv.syringae |
| | | | | ZP_00123737.1,9498aa | 0.0 | 468/470 (99.57%) | 469/470 (99.79%) | hypothetical protein, |
| | | | | | | | | Pseudomonas syringae |
| | | | | | | | | pv.syringae B728a |
| | | | | AAL57600.1,3316aa | 1e − 125 | 234/473 (49.47%) | 310/473 (65.54%) | peptide synthetase XpsB, |
| | | | | | | | | Xenorhabdus bovienii |
| 25 | AAF99707nCD02 | SYRP | 471 | AAF99707.2,5379aa | 0.0 | 446/471 (94.69%) | 446/471 (94.69%) | syringopeptin synthetase, |
| | | | | | | | | Pseudomonas syringae |
| | | | | | | | | pv.syringae |
| | | | | ZP_00123731.1,5345aa | 0.0 | 427/471 (90.66%) | 433/471 (91.93%) | hypothetical protein, |
| | | | | | | | | Pseudomonas syringae |
| | | | | | | | | pv.syringae B728a |
| | | | | ZP_00126973.1,3020aa | 1e − 151 | 273/471 (57.96%) | 331/471 (70.28%) | hypothetical protein, |
| | | | | | | | | Pseudomonas syringae |
| | | | | | | | | pv.syringae B728a |
| 27 | AAF99707nCD03 | SYRP | 466 | AAF99707.2,5379aa | 0.0 | 466/466 (100%) | 466/466 (100%) | syringopeptin synthetase, |
| | | | | | | | | Pseudomonas syringae |
| | | | | | | | | pv.syringae |
| | | | | ZP_00123731.1,5345aa | 0.0 | 460/466 (98.71%) | 463/466 (99.36%) | hypothetical protein, |
| | | | | | | | | Pseudomonas syringae |
| | | | | | | | | pv.syringae B728a |
| | | | | AAO72424.1,5457aa | 1e − 174 | 315/468 (67.31%) | 356/468 (76.07%) | syringopeptin |
| | | | | | | | | synthetase B, |
| | | | | | | | | Pseudomonas syringae |
| | | | | | | | | pv.syringae |
| 29 | AAF99707nCD04 | SYRP | 468 | AAF99707.2,5379aa | 0.0 | 468/468 (100%) | 468/468 (100%) | syringopeptin synthetase, |
| | | | | | | | | Pseudomonas syringae |
| | | | | | | | | pv.syringae |
| | | | | ZP_00123737.1,9498aa | 0.0 | 368/468 (78.63%) | 402/468 (85.9%) | hypothetical protein, |
| | | | | | | | | Pseudomonas syringae |
| | | | | | | | | pv.syringae B728a |
| | | | | T14593,9376aa | 0.0 | 353/468 (75.43%) | 386/468 (82.48%) | syringomycin synthetase, |
| | | | | | | | | Pseudomonas syringae |
| | | | | | | | | pv.syringae |
| 31 | AAF99707nCD05 | SYRP | 468 | AAF99707.2,5379aa | 0.0 | 452/468 (96.58%) | 452/468 (96.58%) | syringopeptin synthetase, |
| | | | | | | | | Pseudomonas syringae |
| | | | | | | | | pv.syringae |
| | | | | ZP_00123731.1,5345aa | 0.0 | 398/468 (85.04%) | 422/468 (90.17%) | hypothetical protein, |
| | | | | | | | | Pseudomonas syringae |
| | | | | | | | | pv.syringae B728a |
| | | | | AAO72425.1,13536aa | 0.0 | 340/470 (72.34%) | 385/470 (81.91%) | syringopeptin |
| | | | | | | | | synthetase C, |
| | | | | | | | | Pseudomonas syringae |
| | | | | | | | | pv.syringae |
| 33 | AAO72424nCD01 | SYRP | 468 | AAO72424.1,5457aa | 0.0 | 451/468 (96.37%) | 451/468 (96.37%) | syringopeptin |
| | | | | | | | | synthetase B, |
| | | | | | | | | Pseudomonas syringae |
| | | | | | | | | pv. Syringae |
| | | | | ZP_00123730.1,1142aa | 0.0 | 419/468 (89.53%) | 430/468 (91.88%) | hypothetical protein, |
| | | | | | | | | Pseudomonas syringae |
| | | | | | | | | pv.syringae B728a |
| | | | | AAO72425.1,13536aa | 0.0 | 323/470 (68.72%) | 365/470 (77.66%) | syringopeptin |
| | | | | | | | | synthetase C, |
| | | | | | | | | Pseudomonas syringae |
| | | | | | | | | pv. Syringae |
| 35 | AAO72424nCD02 | SYRP | 468 | AAO72424.1,5457aa | 0.0 | 458/468 (97.86%) | 458/468 (97.86%) | syringopeptin |
| | | | | | | | | synthetase B, |
| | | | | | | | | Pseudomonas syringae |
| | | | | | | | | pv.syringae |
| | | | | ZP_00128201.1,1970aa | 0.0 | 319/468 (68.16%) | 374/468 (79.91%) | hypothetical protein, |
| | | | | | | | | Pseudomonas syringae |
| | | | | | | | | pv.syringae B728a |
| | | | | ZP_00126883.1,2901aa | 0.0 | 322/468 (68.8%) | 372/468 (79.49%) | hypothetical protein, |
| | | | | | | | | Pseudomonas syringae |
| | | | | | | | | pv.syringae B728a |
| 37 | AAO72424nCD03 | SYRP | 468 | AAO72424.1,5457aa | 0.0 | 401/468 (85.68%) | 401/468 (85.68%) | syringopeptin |
| | | | | | | | | synthetase B, |
| | | | | | | | | Pseudomonas syringae |
| | | | | | | | | pv.syringae |
| | | | | ZP_00128201.1,1970aa | 0.0 | 396/468 (84.62%) | 399/468 (85.26%) | hypothetical protein, |
| | | | | | | | | Pseudomonas syringae |
| | | | | | | | | pv.syringae B728a |
| | | | | AAO72425.1,13536aa | 1e − 164 | 297/470 (63.19%) | 332/470 (70.64%) | syringopeptin |
| | | | | | | | | synthetase C, |
| | | | | | | | | Pseudomonas syringae |
| | | | | | | | | pv.syringae |
| 39 | AAO72424nCD04 | SYRP | 467 | AAO72424.1,5457aa | 0.0 | 467/467 (100%) | 467/467 (100%) | syringopeptin |
| | | | | | | | | synthetase B, |
| | | | | | | | | Pseudomonas syringae |
| | | | | | | | | pv.syringae |
| | | | | ZP_00125933.1,6656aa | 0.0 | 384/469 (81.88%) | 422/469 (89.98%) | hypothetical protein, |
| | | | | | | | | Pseudomonas syringae |
| | | | | | | | | pv.syringae B728a |
| | | | | AAO72425.1,13536aa | 0.0 | 381/466 (81.76%) | 420/466 (90.13%) | syringopeptin |
| | | | | | | | | synthetase C, |
| | | | | | | | | Pseudomonas syringae |
| | | | | | | | | pv.syringae |
| 41 | AAO72424nCD05 | SYRP | 467 | AAO72424.1,5457aa | 0.0 | 467/467 (100%) | 467/467 (100%) | syringopeptin |
| | | | | | | | | synthetase B, |
| | | | | | | | | Pseudomonas syringae |
| | | | | | | | | pv.syringae |
| | | | | ZP_00126884.1,1953aa | 0.0 | 460/467 (98.5%) | 465/467 (99.57%) | hypothetical protein, |
| | | | | | | | | Pseudomonas syringae |
| | | | | | | | | pv.syringae B728a |
| | | | | ZP_00125933.1,6656aa | 0.0 | 418/467 (89.51%) | 440/467 (94.22%) | hypothetical protein, |
| | | | | | | | | Pseudomonas syringae |
| | | | | | | | | pv.syringae B728a |
| 43 | AAO72425nCD01 | SYRP | 469 | AAO72425.1,13536aa | 0.0 | 458/469 (97.65%) | 458/469 (97.65%) | syringopeptin |
| | | | | | | | | synthetase C, |
| | | | | | | | | Pseudomonas syringae |
| | | | | | | | | pv.syringae |
| | | | | ZP_00126883.1,2901aa | 0.0 | 457/469 (97.44%) | 457/469 (97.44%) | hypothetical protein, |
| | | | | | | | | Pseudomonas syringae |
| | | | | | | | | pv.syringae B728a |
| | | | | AAO72424.1,5457aa | 1e − 168 | 294/469 (62.69%) | 357/469 (76.12%) | syringopeptin |
| | | | | | | | | synthetase B, |
| | | | | | | | | Pseudomonas syringae |
| | | | | | | | | pv.syringae |
| 45 | AAO72425nCD02 | SYRP | 468 | AAO72425.1,13536aa | 0.0 | 435/468 (92.95%) | 435/468 (92.95%) | syringopeptin |
| | | | | | | | | synthetase C, |
| | | | | | | | | Pseudomonas syringae |
| | | | | | | | | pv.syringae |
| | | | | ZP_00126883.1,2901aa | 0.0 | 432/468 (92.31%) | 433/468 (92.52%) | hypothetical protein, |
| | | | | | | | | Pseudomonas syringae |
| | | | | | | | | pv.syringae B728a |
| | | | | AAF99707.2,5379aa | 0.0 | 321/468 (68.59%) | 370/468 (79.06%) | syringopeptin synthetase, |
| | | | | | | | | Pseudomonas syringae |
| | | | | | | | | pv.syringae |
| 47 | AAO72425nCD03 | SYRP | 468 | AAO72425.1,13536aa | 0.0 | 448/468 (96.73%) | 448/468 (95.73%) | syringopeptin |
| | | | | | | | | synthetase C, |
| | | | | | | | | Pseudomonas syringae |
| | | | | | | | | pv.syringae |
| | | | | ZP_00126883.1,2901aa | 0.0 | 372/392 (94.9%) | 372/392 (94.9%) | hypothetical protein, |
| | | | | | | | | Pseudomonas syringae |
| | | | | | | | | pv.syringae B728a |
| | | | | AAF99707.2,5379aa | 0.0 | 321/468 (68.59%) | 369/468 (78.85%) | syringopeptin synthetase, |
| | | | | | | | | Pseudomonas syringae |
| | | | | | | | | pv.syringae |
| 49 | AAO72425nCD04 | SYRP | 469 | AAO72425.1,13536aa | 0.0 | 469/469 (100%) | 469/469 (100%) | syringopeptin |
| | | | | | | | | synthetase C, |
| | | | | | | | | Pseudomonas syringae |
| | | | | | | | | pv.syringae |
| | | | | ZP_00126973.1,3020aa | 0.0 | 460/469 (98.08%) | 464/469 (98.93%) | hypothetical protein, |
| | | | | | | | | Pseudomonas syringae |
| | | | | | | | | pv.syringae B728a |
| | | | | ZP_00128393.1,508aa | 0.0 | 355/385 (92.21%) | 371/385 (96.36%) | hypothetical protein, |
| | | | | | | | | Pseudomonas syringae |
| | | | | | | | | pv.syringae B728a |
| 51 | AAO72425nCD05 | SYRP | 466 | AAO72425.1,13536aa | 0.0 | 466/466 (100%) | 466/466(100%) | syringopeptin |
| | | | | | | | | synthetase C, |
| | | | | | | | | Pseudomonas syringae |
| | | | | | | | | pv.syringae |
| | | | | ZP_00126973.1,3020aa | 0.0 | 462/466 (99.14%) | 463/466 (99.36%) | hypothetical protein, |
| | | | | | | | | Pseudomonas syringae |
| | | | | | | | | pv.syringae B728a |
| | | | | AAO72424.1,5457aa | 0.0 | 388/464 (83.62%) | 412/464 (88.79%) | syringopeptin |
| | | | | | | | | synthetase B, |
| | | | | | | | | Pseudomonas syringae |
| | | | | | | | | pv.syringae |
| 53 | AAO72425nCD06 | SYRP | 468 | AAO72425.1,13536aa | 0.0 | 468/468 (100%) | 468/468 (100%) | syringopeptin |
| | | | | | | | | synthetase C, |
| | | | | | | | | Pseudomonas syringae |
| | | | | | | | | pv.syringae |
| | | | | ZP_00126902.1,1463aa | 0.0 | 431/439 (98.18%) | 434/439 (98.86%) | hypothetical protein, |
| | | | | | | | | Pseudomonas syringae |
| | | | | | | | | pv.syringae B728a |
| | | | | ZP_00123737.1,9498aa | 1e − 180 | 319/468 (68.16%) | 372/468 (79.49%) | hypothetical protein, |
| | | | | | | | | Pseudomonas syringae |
| | | | | | | | | pv.syringae B728a |
| 55 | AAO72425nCD07 | SYRP | 470 | AAO72425.1,13536aa | 0.0 | 470/470 (100%) | 470/470 (100%) | syringopeptin |
| | | | | | | | | synthetase C, |
| | | | | | | | | Pseudomonas syringae |
| | | | | | | | | pv.syringae |
| | | | | ZP_00126883.1,2901aa | 0.0 | 397/470 (84.47%) | 433/470 (92.13%) | hypothetical protein, |
| | | | | | | | | Pseudomonas syringae |
| | | | | | | | | pv.syringae B728a |
| | | | | ZP_00126902.1,1463aa | 0.0 | 384/393 (97.71%) | 387/393 (98.47%) | hypothetical protein, |
| | | | | | | | | Pseudomonas syringae |
| | | | | | | | | pv.syringae B728a |
| 57 | AAO72425nCD08 | SYRP | 467 | AAO72425.1,13536aa | 0.0 | 440/467 (94.22%) | 440/467 (94.22%) | syringopeptin |
| | | | | | | | | synthetase C, |
| | | | | | | | | Pseudomonas syringae |
| | | | | | | | | pv.syringae |
| | | | | ZP_00125933.1,6656aa | 0.0 | 437/467 (93.58%) | 440/467 (94.22%) | hypothetical protein, |
| | | | | | | | | Pseudomonas syringae |
| | | | | | | | | pv.syringae B728a |
| | | | | AAO72424.1,5457aa | 0.0 | 361/466 (77.47%) | 398/466 (85.41%) | syringopeptin |
| | | | | | | | | synthetase B, |
| | | | | | | | | Pseudomonas syringae |
| | | | | | | | | pv.syringae |
| 59 | AAO72425nCD09 | SYRP | 467 | AAO72425.1,13536aa | 0.0 | 467/467 (100%) | 467/467 (100%) | syringopeptin |
| | | | | | | | | synthetase C, |
| | | | | | | | | Pseudomonas syringae |
| | | | | | | | | pv.syringae |
| | | | | ZP_00125933.1,6656aa | 0.0 | 452/467 (96.79%) | 458/467 (98.07%) | hypothetical protein, |
| | | | | | | | | Pseudomonas syringae |
| | | | | | | | | pv.syringae B728a |
| | | | | ZP_00126884.1,1953aa | 0.0 | 420/467 (89.94%) | 437/467 (93.58%) | hypothetical protein, |
| | | | | | | | | Pseudomonas syringae |
| | | | | | | | | pv.syringae B728a |
| 61 | CAD17793nCD01 | URSO | 468 | NP_522203.1,5953aa | 0.0 | 432/468 (92.31%) | 432/468 (92.31%) | probable peptide |
| | | | | | | | | synthase, |
| | | | | | | | | Ralstonia solanacearum |
| | | | | ZP_00123730.1,1142aa | 1e − 148 | 276/469 (58.85%) | 325/469 (69.3%) | hypothetical protein, |
| | | | | | | | | Pseudomonas syringae |
| | | | | | | | | pv.syringae B728a |
| | | | | AAO72424.1,5457aa | 1e − 147 | 272/469 (58%) | 326/469 (69.51%) | syringopeptin |
| | | | | | | | | synthetase B, |
| | | | | | | | | Pseudomonas syringae |
| | | | | | | | | pv.syringae |
| 63 | CAD17793nCD03 | URSO | 469 | NP_522203.1,5953aa | 0.0 | 451/469 (96.16%) | 451/469 (96.16%) | probable peptide |
| | | | | | | | | synthase, |
| | | | | | | | | Ralstonia solanacearum |
| | | | | AAO72424.1,5457aa | 1e − 133 | 249/469 (53.09%) | 316/469 (67.38%) | syringopeptin |
| | | | | | | | | synthetase B, |
| | | | | | | | | Pseudomonas syringae |
| | | | | | | | | pv.syringae |
| | | | | ZP_00126883.1,2901aa | 1e − 133 | 248/469 (52.88%) | 314/469 (66.95%) | hypothetical protein, |
| | | | | | | | | Pseudomonas syringae |
| | | | | | | | | pv.syringae B728a |
| 65 | 023CCP_01nCD02 | 023C | 469 | AAF99707.2,5379aa | 4e − 94 | 195/472 (41.31%) | 269/472 (56.99%) | syringopeptin synthetase, |
| | | | | | | | | Pseudomonas syringae |
| | | | | | | | | pv.syringae |
| | | | | ZP_00086025.1,5422aa | 2e − 93 | 202/472 (42.8%) | 266/472 (56.36%) | hypothetical protein, |
| | | | | | | | | Pseudomonas fluorescens |
| | | | | | | | | PfO-1 |
| | | | | ZP_00123731.1,5345aa | 5e − 92 | 197/472 (41.74%) | 266/472 (56.36%) | hypothetical protein, |
| | | | | | | | | Pseudomonas syringae |
| | | | | | | | | pv.syringae B728a |
| 67 | 023CCP_01nCD03 | 023C | 472 | ZP_00126883.1,2901aa | 1e − 102 | 209/475 (44%) | 279/475 (58.74%) | hypothetical protein, |
| | | | | | | | | Pseudomonas syringae |
| | | | | | | | | pv.syringae B728a |
| | | | | AAO72425.1,13536aa | 1e − 101 | 208/475 (43.79%) | 278/475 (58.53%) | syringopeptin |
| | | | | | | | | synthetase C, |
| | | | | | | | | Pseudomonas syringae |
| | | | | | | | | pv.syringae |
| | | | | AAF99707.2,5379aa | 7e − 97 | 202/475 (42.53%) | 277/475 (58.32%) | syringopeptin synthetase, |
| | | | | | | | | Pseudomonas syringae |
| | | | | | | | | pv.syringae |
| 69 | 023CCP_06nCD02 | 023C | 480 | AAF99707.2,5379aa | 9e − 94 | 197/481 (40.96%) | 277/481 (57.59%) | syringopeptin synthetase, |
| | | | | | | | | Pseudomonas syringae |
| | | | | | | | | pv.syringae |
| | | | | AAO72424.1,5457aa | 4e − 93 | 198/481 (41.16%) | 274/481 (56.96%) | syringopeptin |
| | | | | | | | | synthetase B, |
| | | | | | | | | Pseudomonas syringae |
| | | | | | | | | pv.syringae |
| | | | | AAO72425.1,13536aa | 3e − 90 | 192/481 (39.92%) | 271/481 (56.34%) | syringopeptin |
| | | | | | | | | synthetase C, |
| | | | | | | | | Pseudomonas syringae |
| | | | | | | | | pv.syringae |
| 71 | 023CSPF10nCD01 | 023C | 466 | NP_522203.1,5953aa | 1e − 95 | 201/477 (42.14%) | 277/477 (58.07%) | probable peptide |
| | | | | | | | | synthase, |
| | | | | | | | | Ralstonia solanacearum |
| | | | | ZP_00086025.1,5422aa | 2e − 95 | 202/472 (42.8%) | 276/472 (58.47%) | hypothetical protein, |
| | | | | | | | | Pseudomonas fluorescens |
| | | | | | | | | PfO-1 |
| | | | | AAO72425.1,13536aa | 6e − 95 | 198/472 (41.95%) | 276/472 (58.47%) | syringopeptin |
| | | | | | | | | synthetase C, |
| | | | | | | | | Pseudomonas syringae |
| | | | | | | | | pv.syringae |
| 73 | 023CSPF10nCD02 | 023C | 468 | AAF99707.2,5379aa | 1e − 107 | 211/472 (44.7%) | 291/472 (61.65%) | syringopeptin synthetase |
| | | | | | | | | Pseudomonas syringae |
| | | | | | | | | pv.syringae |
| | | | | ZP_00126883.1,2901aa | 1e − 104 | 219/472 (46.4%) | 287/472 (60.81%) | hypothetical protein, |
| | | | | | | | | Pseudomonas syringae |
| | | | | | | | | pv.syringae B728a |
| | | | | AAO72425.1,13536aa | 1e − 104 | 217/472 (45.97%) | 284/472 (60.17%) | syringopeptin |
| | | | | | | | | synthetase C, |
| | | | | | | | | Pseudomonas syringae |
| | | | | | | | | pv.syringae |
| 75 | 023CSPF10nCD03 | 023C | 469 | AAO72425.1,13536aa | 1e − 96 | 198/472 (41.95%) | 287/472 (60.81%) | syringopeptin |
| | | | | | | | | synthetase C, |
| | | | | | | | | Pseudomonas syringae |
| | | | | | | | | pv.syringae |
| | | | | AAF99707.2,5379aa | 4e − 94 | 201/472 (42.58%) | 283/472 (59.96%) | syringopeptin synthetase, |
| | | | | | | | | Pseudomonas syringae |
| | | | | | | | | pv.syringae |
| | | | | AAO72424.1,5457aa | 2e − 93 | 198/475 (41.68%) | 279/475 (58.74%) | syringopeptin |
| | | | | | | | | synthetase B, |
| | | | | | | | | Pseudomonas syringae |
| | | | | | | | | pv.syringae |
| 77 | 023CSP_11nCD03 | 023C | 469 | ZP_00086025.1,5422aa | 7e − 96 | 204/474 (43.04%) | 276/474 (58.23%) | hypothetical protein, |
| | | | | | | | | Pseudomonas fluorescens |
| | | | | | | | | PfO-1 |
| | | | | ZP_00126883.1,2901aa | 2e − 95 | 195/472 (41.31%) | 279/472 (59.11%) | hypothetical protein, |
| | | | | | | | | Pseudomonas syringae |
| | | | | | | | | pv.syringae B728a |
| | | | | AAO72425.1,13536aa | 3e − 95 | 194/474 (40.93%) | 279/474 (58.86%) | syringopeptin |
| | | | | | | | | synthetase C, |
| | | | | | | | | Pseudomonas syringae |
| | | | | | | | | pv.syringae |
| 79 | 023CSP_11nCD04 | 023C | 472 | AAO72425.1,13536aa | 1e − 97 | 200/475 (42.11%) | 276/475 (58.11%) | syringopeptin |
| | | | | | | | | synthetase C, |
| | | | | | | | | Pseudomonas syringae |
| | | | | | | | | pv.syringae |
| | | | | ZP_00126883.1,2901aa | 2e − 97 | 200/475 (42.11%) | 275/475 (57.89%) | hypothetical protein, |
| | | | | | | | | Pseudomonas syringae |
| | | | | | | | | pv.syringae B728a |
| | | | | AAO72424.1,5457aa | 3e − 96 | 202/475 (42.53%) | 277/475 (58.32%) | syringopeptin |
| | | | | | | | | synthetase B, |
| | | | | | | | | Pseudomonas syringae |
| | | | | | | | | pv.syringae |
| 81 | 023CSP_11nCD05 | 023C | 473 | AAO72424.1,5457aa | 1e − 103 | 211/475 (44.42%) | 279/475 (58.74%) | syringopeptin |
| | | | | | | | | synthetase B, |
| | | | | | | | | Pseudomonas syringae |
| | | | | | | | | pv.syringae |
| | | | | AAF99707.2,5379aa | 1e − 103 | 211/475 (44.42%) | 286/475 (60.21%) | syringopeptin synthetase, |
| | | | | | | | | Pseudomonas syringae |
| | | | | | | | | pv.syringae |
| | | | | AAO72425.1,13536aa | 1e − 102 | 206/475 (43.37%) | 283/475 (59.58%) | syringopeptin |
| | | | | | | | | synthetase C, |
| | | | | | | | | Pseudomonas syringae |
| | | | | | | | | pv.syringae |
| 83 | 023CYP_01nCD02 | 023C | 476 | NP_522203.1,5953aa | 2e − 93 | 199/480 (41.46%) | 270/480 (56.25%) | probable peptide |
| | | | | | | | | synthase, |
| | | | | | | | | Ralstonia solanacearum |
| | | | | AAO72424.1,5457aa | 3e − 93 | 200/484 (41.32%) | 275/484 (56.82%) | syringopeptin |
| | | | | | | | | synthetase B, |
| | | | | | | | | Pseudomonas syringae |
| | | | | | | | | pv.syringae |
| | | | | AAO72425.1,13536aa | 3e − 91 | 200/483 (41.41%) | 269/483 (55.69%) | syringopeptin |
| | | | | | | | | synthetase C, |
| | | | | | | | | Pseudomonas syringae |
| | | | | | | | | pv.syringae |
| 85 | 023CYP_01nCD03 | 023C | 479 | AAO72425.1,13536aa | 1e − 110 | 220/483 (45.55%) | 306/483 (63.35%) | syringopeptin |
| | | | | | | | | synthetase C, |
| | | | | | | | | Pseudomonas syringae |
| | | | | | | | | pv.syringae |
| | | | | ZP_00086025.1,5422aa | 1e − 109 | 226/483 (46.79%) | 307/483 (63.56%) | hypothetical protein, |
| | | | | | | | | Pseudomonas fluorescens |
| | | | | | | | | PfO-1 |
| | | | | ZP_00126884.1,1953aa | 1e − 109 | 229/483 (47.41%) | 303/483 (62.73%) | hypothetical protein, |
| | | | | | | | | Pseudomonas syringae |
| | | | | | | | | pv.syringae B728a |
| 87 | 023CYP_02nCD02 | 023C | 482 | AAF99707.2,5379aa | 3e − 99 | 204/485 (42.06%) | 284/485 (58.56%) | syringopeptin synthetase, |
| | | | | | | | | Pseudomonas syringae |
| | | | | | | | | pv.syringae |
| | | | | AAO72425.1,13536aa | 1e − 97 | 211/487 (43.33%) | 280/487 (57.49%) | syringopeptin |
| | | | | | | | | synthetase C, |
| | | | | | | | | Pseudomonas syringae |
| | | | | | | | | pv.syringae |
| | | | | BAC67535.1,4338aa | 9e − 97 | 207/485 (42.68%) | 277/485 (57.11%) | arthrofactin |
| | | | | | | | | synthetase B, |
| | | | | | | | | Pseudomonas sp. MIS38 |
| 89 | 023CYP_02nCD03 | 023C | 477 | AAF99707.2,5379aa | 2e − 99 | 201/480 (41.88%) | 285/480 (59.38%) | syringopeptin synthetase |
| | | | | | | | | Pseudomonas syringae |
| | | | | | | | | pv.syringae |
| | | | | AAO72424.1,5457aa | 3e − 99 | 207/480 (43.13%) | 280/480 (58.33%) | syringopeptin |
| | | | | | | | | synthetase B, |
| | | | | | | | | Pseudomonas syringae |
| | | | | | | | | pv.syringae |
| | | | | AAO72425,1,13536aa | 2e − 98 | 196/480 (40.83%) | 285/480 (59.38%) | syringopeptin |
| | | | | | | | | synthetase C, |
| | | | | | | | | Pseudomonas syringae |
| | | | | | | | | pv.syringae |
| 91 | 034CMP_77nCD05 | 034F | 467 | AAO72424.1,5457aa | 1e − 107 | 212/449 (47.22%) | 281/449 (62.58%) | syringopeptin |
| | | | | | | | | synthetase B, |
| | | | | | | | | Pseudomonas syringae |
| | | | | | | | | pv.syringae |
| | | | | ZP_00086025.1,5422aa | 1e − 106 | 217/470 (46.17%) | 288/470 (61.28%) | hypothetical protein, |
| | | | | | | | | Pseudomonas fluorescens |
| | | | | | | | | PfO-1 |
| | | | | ZP_00123731.1,5345aa | 1e − 106 | 211/470 (44.89%) | 281/470 (59.79%) | hypothetical protein, |
| | | | | | | | | Pseudomonas syringae |
| | | | | | | | | pv.syringae B728a |
| 93 | 034CMP_77nCD06 | 034F | 467 | ZP_00086025.1,5422aa | 1e − 116 | 228/470 (48.51%) | 309/470 (65.74%) | hypothetical protein, |
| | | | | | | | | Pseudomonas fluorescens |
| | | | | | | | | PfO-1 |
| | | | | NP_522203.1,5953aa | 1e − 113 | 231/472 (48.94%) | 299/472 (63.35%) | probable peptide |
| | | | | | | | | synthase, |
| | | | | | | | | Ralstonia solanacearum |
| | | | | AAF99707.2,5379aa | 1e − 112 | 222/470 (47.23%) | 305/470 (64.89%) | syringopeptin synthetase, |
| | | | | | | | | Pseudomonas syringae |
| | | | | | | | | pv.syringae |
| 95 | 034CMP_76nCD02 | 034F | 467 | ZP_00086025.1,5422aa | 1e − 115 | 229/471 (48.62%) | 306/471 (64.97%) | hypothetical protein, |
| | | | | | | | | Pseudomonas fluorescens |
| | | | | | | | | PfO-1 |
| | | | | AAF99707.2,5379aa | 1e − 114 | 223/470 (47.45%) | 302/470 (64.26%) | syringopeptin synthetase, |
| | | | | | | | | Pseudomonas syringae |
| | | | | | | | | pv.syringae |
| | | | | ZP_00126388.1,3699aa | 1e − 113 | 232/471 (49.26%) | 295/471 (62.63%) | hypothetical protein, |
| | | | | | | | | Pseudomonas syringae |
| | | | | | | | | pv.syringae B728a |
| 97 | 034CMP_76nCD04 | 034F | 467 | AAF99707.2,5379aa | 1e − 116 | 227/470 (48.3%) | 295/470 (62.77%) | syringopeptin synthetase, |
| | | | | | | | | Pseudomonas syringae |
| | | | | | | | | pv.syringae |
| | | | | ZP_00123731.1,5345aa | 1e − 113 | 220/470 (46.81%) | 292/470 (62.13%) | hypothetical protein, |
| | | | | | | | | Pseudomonas syringae |
| | | | | | | | | pv.syringae B728a |
| | | | | NP_522203.1,5953aa | 1e − 111 | 231/472 (48.94%) | 294/472 (62.29%) | probable peptide |
| | | | | | | | | synthase, |
| | | | | | | | | Ralstonia solanacearum |
| 99 | 040CRP_21nCD03 | 040G | 462 | AAF99707.2,5379aa | 1e − 112 | 224/468 (47.86%) | 291/468 (62.18%) | syringopeptin synthetase, |
| | | | | | | | | Pseudomonas syringae |
| | | | | | | | | pv.syringae |
| | | | | ZP_00123731.1,5345aa | 1e − 110 | 226/468 (48.29%) | 289/468 (61.75%) | hypothetical protein, |
| | | | | | | | | Pseudomonas syringae |
| | | | | | | | | pv.syringae B728a |
| | | | | ZP_00086025.1,5422aa | 1e − 110 | 227/470 (48.3%) | 296/470 (62.98%) | hypothetical protein, |
| | | | | | | | | Pseudomonas fluorescens |
| | | | | | | | | PfO-1 |
| 101 | 040CRP_21nCD07 | 040G | 463 | AAF99707.2,5379aa | 1e − 121 | 239/470 (50.85%) | 304/470 (64.68%) | syringopeptin synthetase, |
| | | | | | | | | Pseudomonas syringae |
| | | | | | | | | pv.syringae |
| | | | | ZP_00123731.1,5345aa | 1e − 118 | 236/470 (50.21%) | 303/470 (64.47%) | hypothetical protein, |
| | | | | | | | | Pseudomonas syringae |
| | | | | | | | | pv.syringae B728a |
| | | | | ZP_00086026.1,4882aa | 1e − 117 | 229/469 (48.83%) | 296/469 (63.11%) | hypothetical protein, |
| | | | | | | | | Pseudomonas fluorescens |
| | | | | | | | | PfO-1 |
| 103 | 084CBP_48nCD02 | 084B | 464 | ZP_00126973.1,3020aa | 1e − 109 | 219/469 (46.7%) | 290/469 (61.83%) | hypothetical protein, |
| | | | | | | | | Pseudomonas syringae |
| | | | | | | | | pv.syringae B728a |
| | | | | AAO72425.1,13536aa | 1e − 109 | 218/469 (46.48%) | 289/469 (61.62%) | syringopeptin |
| | | | | | | | | synthetase C, |
| | | | | | | | | Pseudomonas syringae |
| | | | | | | | | pv.syringae |
| | | | | AAO72424.1,5457aa | 1e − 107 | 217/468 (46.37%) | 292/468 (62.39%) | syringopeptin |
| | | | | | | | | synthetase B, |
| | | | | | | | | Pseudomonas syringae |
| | | | | | | | | pv.syringae |
| 105 | 084CBP_48nCD03 | 084B | 470 | ZP_00126973.1,3020aa | 1e − 101 | 200/443 (45.15%) | 273/443 (61.63%) | hypothetical protein, |
| | | | | | | | | Pseudomonas syringae |
| | | | | | | | | pv.syringae B728a |
| | | | | ZP_00126884.1,1953aa | 1e − 101 | 199/440 (45.23%) | 277/440 (62.95%) | hypothetical protein, |
| | | | | | | | | Pseudomonas syringae |
| | | | | | | | | pv.syringae B728a |
| | | | | ZP_00086025.1,5422aa | 1e − 101 | 198/443 (44.7%) | 273/443 (61.63%) | hypothetical protein, |
| | | | | | | | | Pseudomonas fluorescens |
| | | | | | | | | PfO-1 |
| 107 | 084CBP_48nCD04 | 084B | 465 | AAO72424.1,5457aa | 1e − 106 | 218/468 (46.58%) | 285/468 (60.9%) | syringopeptin |
| | | | | | | | | synthetase B, |
| | | | | | | | | Pseudomonas syringae |
| | | | | | | | | pv.syringae |
| | | | | AAO72425.1,13536aa | 1e − 104 | 212/470 (45.11%) | 284/470 (60.43%) | syringopeptin |
| | | | | | | | | synthetase C, |
| | | | | | | | | Pseudomonas syringae |
| | | | | | | | | pv.syringae |
| | | | | ZP_00126883.1,2901aa | 1e − 104 | 212/470 (45.11%) | 284/470 (60.43%) | hypothetical protein, |
| | | | | | | | | Pseudomonas syringae |
| | | | | | | | | pv.syringae B728a |
| 109 | 084CBP_48nCD05 | 084B | 463 | AAF99707.2,5379aa | 1e − 102 | 194/468 (41.45%) | 276/468 (58.97%) | syringopeptin synthetase, |
| | | | | | | | | Pseudomonas syringae |
| | | | | | | | | pv.syringae |
| | | | | ZP_00126884.1,1953aa | 1e − 102 | 205/467 (43.9%) | 280/467 (59.96%) | hypothetical protein, |
| | | | | | | | | Pseudomonas syringae |
| | | | | | | | | pv.syringae B728a |
| | | | | AAO72424.1,5457aa | 1e − 100 | 203/467 (43.47%) | 280/467 (59.96%) | syringopeptin |
| | | | | | | | | synthetase B, |
| | | | | | | | | Pseudomonas syringae |
| | | | | | | | | pv.syringae |
| 111 | 107CAPC02nCD01 | 107A | 466 | NP_522203.1,5953aa | 5e − 98 | 208/473 (43.97%) | 274/473 (57.93%) | probable peptide |
| | | | | | | | | synthase, |
| | | | | | | | | Ralstonia solanacearum |
| | | | | ZP_00086026.1,4882aa | 1e − 97 | 203/470 (43.19%) | 267/470 (56.81%) | hypothetical protein, |
| | | | | | | | | Pseudomonas fluorescens |
| | | | | | | | | PfO-1 |
| | | | | AAF99707.2,5379aa | 1e − 96 | 199/472 (42.16%) | 273/472 (57.84%) | syringopeptin synthetase, |
| | | | | | | | | Pseudomonas syringae |
| | | | | | | | | pv.syringae |
| 113 | 107CAP_12nCD01 | 107A | 466 | BAC67534.1,2040aa | 2e − 96 | 205/472 (43.43%) | 277/472 (58.69%) | arthrofactin synthetase A |
| | | | | | | | | Pseudomonas sp. MIS38 |
| | | | | ZP_00086026.1,4882aa | 6e − 96 | 203/470 (43.19%) | 267/470 (56.81%) | hypothetical protein, |
| | | | | | | | | Pseudomonas fluorescens |
| | | | | | | | | PfO-1 |
| | | | | AAF99707.2,5379aa | 4e − 95 | 198/472 (41.95%) | 272/472 (57.63%) | syringopeptin synthetase, |
| | | | | | | | | Pseudomonas syringae |
| | | | | | | | | pv.syringae |
| 115 | 143KKP_39nCD02 | 143F | 464 | AAO72425.1,13536aa | 4e − 91 | 187/441 (42.4%) | 259/441 (58.73%) | syringopeptin |
| | | | | | | | | synthetase C, |
| | | | | | | | | Pseudomonas syringae |
| | | | | | | | | pv.syringae |
| | | | | ZP_00086025.1,5422aa | 4e − 89 | 194/440 (44.09%) | 247/440 (56.14%) | hypothetical protein, |
| | | | | | | | | Pseudomonas fluorescens |
| | | | | | | | | PfO-1 |
| | | | | ZP_00123737.1,9498aa | 2e − 87 | 190/441 (43.08%) | 244/441 (55.33%) | hypothetical protein, |
| | | | | | | | | Pseudomonas syringae |
| | | | | | | | | pv.syringae B728a |
| 117 | 143KKP_39nCD06 | 143F | 467 | ZP_00123731.1,5345aa | 1e − 115 | 233/471 (49.47%) | 304/471 (64.54%) | hypothetical protein, |
| | | | | | | | | Pseudomonas syringae |
| | | | | | | | | pv.syringae B728a |
| | | | | AAF99707.2,5379aa | 1e − 113 | 227/473 (47.99%) | 301/473 (63.64%) | syringopeptin synthetase, |
| | | | | | | | | Pseudomonas syringae |
| | | | | | | | | pv.syringae |
| | | | | AAO72425.1,13536aa | 1e − 112 | 230/470 (48.94%) | 296/470 (62.98%) | syringopeptin |
| | | | | | | | | synthetase C, |
| | | | | | | | | Pseudomonas syringae |
| | | | | | | | | pv.syringae |
| 119 | 143KKP_40nCD03 | 143F | 461 | AAF99707.2,5379aa | 1e − 114 | 223/468 (47.65%) | 299/468 (63.89%) | syringopeptin synthetase, |
| | | | | | | | | Pseudomonas syringae |
| | | | | | | | | pv.syringae |
| | | | | ZP_00123731.1,5345aa | 1e − 114 | 227/470 (48.3%) | 302/470 (64.26%) | hypothetical protein, |
| | | | | | | | | Pseudomonas syringae |
| | | | | | | | | pv.syringae B728a |
| | | | | ZP_00086025.1,5422aa | 1e − 113 | 230/468 (49.15%) | 303/468 (64.74%) | hypothetical protein, |
| | | | | | | | | Pseudomonas fluorescens |
| | | | | | | | | PfO-1 |
| 121 | 143KKP_40nCD04 | 143F | 464 | ZP_0012373.1,15345aa | 1e − 122 | 245/473 (51.8%) | 309/473 (65.33%) | hypothetical protein, |
| | | | | | | | | Pseudomonas syringae |
| | | | | | | | | pv.syringae B728a |
| | | | | ZP_00086025.1,5422aa | 1e − 120 | 245/469 (52.24%) | 312/469 (66.52%) | hypothetical protein, |
| | | | | | | | | Pseudomonas fluorescens |
| | | | | | | | | PfO-1 |
| | | | | AAF99707.2,5379aa | 1e − 119 | 241/473 (50.95%) | 305/473 (64.48%) | syringopeptin synthetase, |
| | | | | | | | | Pseudomonas syringae |
| | | | | | | | | pv.syringae |
| 123 | 153CAP_10nCD02 | 153A | 478 | ZP_00086025.1,5422aa | 8e − 95 | 202/481 (42%) | 272/481 (56.55%) | hypothetical protein, |
| | | | | | | | | Pseudomonas fluorescens |
| | | | | | | | | PfO-1 |
| | | | | ZP_00126883.1,2901aa | 9e − 94 | 190/419 (45.35%) | 249/419 (59.43%) | hypothetical protein, |
| | | | | | | | | Pseudomonas syringae |
| | | | | | | | | pv.syringae B728a |
| | | | | AAO72425.1,13536aa | 3e − 93 | 189/419 (45.11%) | 248/419 (59.19%) | syringopeptin |
| | | | | | | | | synthetase C, |
| | | | | | | | | Pseudomonas syringae |
| | | | | | | | | pv.syringae |
| 125 | 153CAP_10nCD03 | 153A | 468 | AAO72425.1,13536aa | 1e − 102 | 212/471 (45.01%) | 280/471 (59.45%) | syringopeptin |
| | | | | | | | | synthetase C, |
| | | | | | | | | Pseudomonas syringae |
| | | | | | | | | pv.syringae |
| | | | | ZP_00126883.1,2901aa | 1e − 102 | 212/471 (45.01%) | 280/471 (59.45%) | hypothetical protein, |
| | | | | | | | | Pseudomonas syringae |
| | | | | | | | | pv.syringae B728a |
| | | | | AAF99707.2,5379aa | 1e − 98 | 198/471 (42.04%) | 279/471 (59.24%) | syringopeptin synthetase, |
| | | | | | | | | Pseudomonas syringae |
| | | | | | | | | pv.syringae |
| 127 | 153CAP_10nCD04 | 153A | 473 | ZP_00126884.1,1953aa | 4e − 92 | 197/477 (41.3%) | 266/477 (55.77%) | hypothetical protein, |
| | | | | | | | | Pseudomonas syringae |
| | | | | | | | | pv.syringae B728a |
| | | | | ZP_00086025.1,5422aa | 4e − 92 | 191/476 (40.13%) | 275/476 (57.77%) | hypothetical protein, |
| | | | | | | | | Pseudomonas fluorescens |
| | | | | | | | | PfO-1 |
| | | | | AAO72424.1,5457aa | 5e − 92 | 196/477 (41.09%) | 267/477 (55.97%) | syringopeptin |
| | | | | | | | | synthetase B, |
| | | | | | | | | Pseudomonas syringae |
| | | | | | | | | pv.syringae |
| 129 | 153CAP_13nCD02 | 153A | 471 | ZP_00126883.1,2901aa | 1e − 101 | 206/477 (43.19%) | 279/477 (58.49%) | hypothetical protein, |
| | | | | | | | | Pseudomonas syringae |
| | | | | | | | | pv.syringae B728a |
| | | | | AAO72425.1,13536aa | 1e − 100 | 205/477 (42.98%) | 278/477 (58.28%) | syringopeptin |
| | | | | | | | | synthetase C, |
| | | | | | | | | Pseudomonas syringae |
| | | | | | | | | pv.syringae |
| | | | | ZP_00086025.1,5422aa | 2e − 98 | 208/477 (43.61%) | 290/477 (60.8%) | hypothetical protein, |
| | | | | | | | | Pseudomonas fluorescens |
| | | | | | | | | PfO-1 |
| 131 | 263CRPN18nCD01 | 263B | 459 | AAF99707.2,5379aa | 1e − 107 | 214/469 (45.63%) | 296/469 (63.11%) | syringopeptin synthetase, |
| | | | | | | | | Pseudomonas syringae |
| | | | | | | | | pv.syringae |
| | | | | ZP_00123731.1,5345aa | 1e − 103 | 210/469 (44.78%) | 287/469 (61.19%) | hypothetical protein, |
| | | | | | | | | Pseudomonas syringae |
| | | | | | | | | pv.syringae B728a |
| | | | | AAO72424.1,5457aa | 1e − 100 | 206/469 (43.92%) | 283/469 (60.34%) | syringopeptin |
| | | | | | | | | synthetase B, |
| | | | | | | | | Pseudomonas syringae |
| | | | | | | | | pv.syringae |
| 133 | 263CRPN18nCD02 | 263B | 460 | AAO72424.1,5457aa | 1e − 108 | 209/442 (47.29%) | 280/442 (63.35%) | syringopeptin |
| | | | | | | | | synthetase B, |
| | | | | | | | | Pseudomonas syringae |
| | | | | | | | | pv.syringae |
| | | | | ZP_00126883.1,2901aa | 1e − 102 | 204/446 (45.74%) | 272/446 (60.99%) | hypothetical protein, |
| | | | | | | | | Pseudomonas syringae |
| | | | | | | | | pv.syringae B728a |
| | | | | AAO72425.1,13536aa | 1e − 102 | 204/446 (45.74%) | 271/446 (60.76%) | syringopeptin |
| | | | | | | | | synthetase C, |
| | | | | | | | | Pseudomonas syringae |
| | | | | | | | | pv.syringae |
| 135 | 263CRPN44nCD02 | 263B | 466 | AAF99707.2,5379aa | 5e − 99 | 201/473 (42.49%) | 286/473 (60.47%) | syringopeptin synthetase, |
| | | | | | | | | Pseudomonas syringae |
| | | | | | | | | pv.syringae |
| | | | | ZP_00126388.1,3699aa | 2e − 96 | 206/472 (43.64%) | 283/472 (59.96%) | hypothetical protein, |
| | | | | | | | | Pseudomonas syringae |
| | | | | | | | | pv.syringae B728a |
| | | | | ZP_00086025.1,5422aa | 3e − 96 | 198/474 (41.77%) | 280/474 (59.07%) | hypothetical protein, |
| | | | | | | | | Pseudomonas fluorescens |
| | | | | | | | | PfO-1 |
| 137 | 263CRP_46nCD02 | 263B | 460 | AAF99707.2,5379aa | 1e − 87 | 190/464 (40.95%) | 256/464 (55.17%) | syringopeptin synthetase, |
| | | | | | | | | Pseudomonas syringae |
| | | | | | | | | pv.syringae |
| | | | | AAO72425.1,13536aa | 2e − 87 | 190/464 (40.95%) | 252/464 (54.31%) | syringopeptin |
| | | | | | | | | synthetase C, |
| | | | | | | | | Pseudomonas syringae |
| | | | | | | | | pv.syringae |
| | | | | ZP_00125933.1,6656aa | 3e − 86 | 189/464 (40.73%) | 250/464 (53.88%) | hypothetical protein, |
| | | | | | | | | Pseudomonas syringae |
| | | | | | | | | pv.syringae B728a |
| 139 | consensus | | 474 | AAF99707.2,5379aa | 1e − 131 | 251/474 (52.95%) | 304/474 (64.14%) | syringopeptin synthetase, |
| | | | | | | | | Pseudomonas syringae |
| | | | | | | | | pv.syringae |
| | | | | AAO72424.1,5457aa | 1e − 128 | 249/474 (52.53%) | 300/474 (63.29%) | syringopeptin |
| | | | | | | | | synthetase B, |
| | | | | | | | | Pseudomonas syringae |
| | | | | | | | | pv.syringae |
| | | | | AAO72425.1,13536aa | 1e − 127 | 248/476 (52.1%) | 303/476 (63.66%) | syringopeptin |
| | | | | | | | | synthetase C, |
| | | | | | | | | Pseudomonas syringae |
| | | | | | | | | pv.syringae |

The availability of such specialized C domains will prove to be valuable genetic engineering tools for combinatorial construction of hybrid NRPS modules. These hybrid NRPS modules may then be used to modify the stereochemistry of a synthesized peptide compound. For example, a regular condensation domain of a given NRPS system may be replaced with a dual condensation/epimerization NRPS domain selected so as to alter the stereochemistry of polypeptides at specific amino acid sites.

Recombinant NRPS systems may be employed either in vivo, using an appropriate recombinant host, or in vitro, using purified enzymes supplemented with the appropriate substrates.

The use of dual condensation/epimerization NRPS domains for modifying the stereochemistry of peptide compounds presents major advantages over insertion of canonical epimerization domains in specific NRPS locations. Insertion of foreign domains in the NRPS complex may interfere with the activities of existing domains. However, use of dual condensation/epimerization NRPS domains instead of epimerization domains to specifically alter the stereochemistry of peptide compounds is expected to alleviate this problem as both regular condensation domains and dual condensation/epimerization NRPS domains are structurally similar.

### EXAMPLE 1

### Presence of Dual Condensation/Epimerization Domains in the Ramoplanin Non-ribosomal Peptide Synthetase (NRPS) Biosynthetic System

Ramoplanin is a lipopeptide produced by Actinoplanes sp. ATCC 33076 (see U.S. Pat. No. 4,303,646). Ramoplanin is a glycosylated lipodepsipeptide of known structure (see, for example, U.S. Pat. No. 4,427,656). The full-length biosynthetic locus for ramoplanin from Actinoplanes sp., referred to herein as RAMO, was cloned and sequenced using the genome scanning method as described by Zazopoulos et al. (2003), Nature Biotechnol, 21, 187-190. The open reading frames in RAMO were identified and a function was attributed to each protein encoded by the open reading frames. RAMO is described in detail in co-pending US application U.S. Ser. No. 09/976,059 and in PCT international application PCT/CA01/01462, published as WO 02/31155, both of which are incorporated herein by reference.

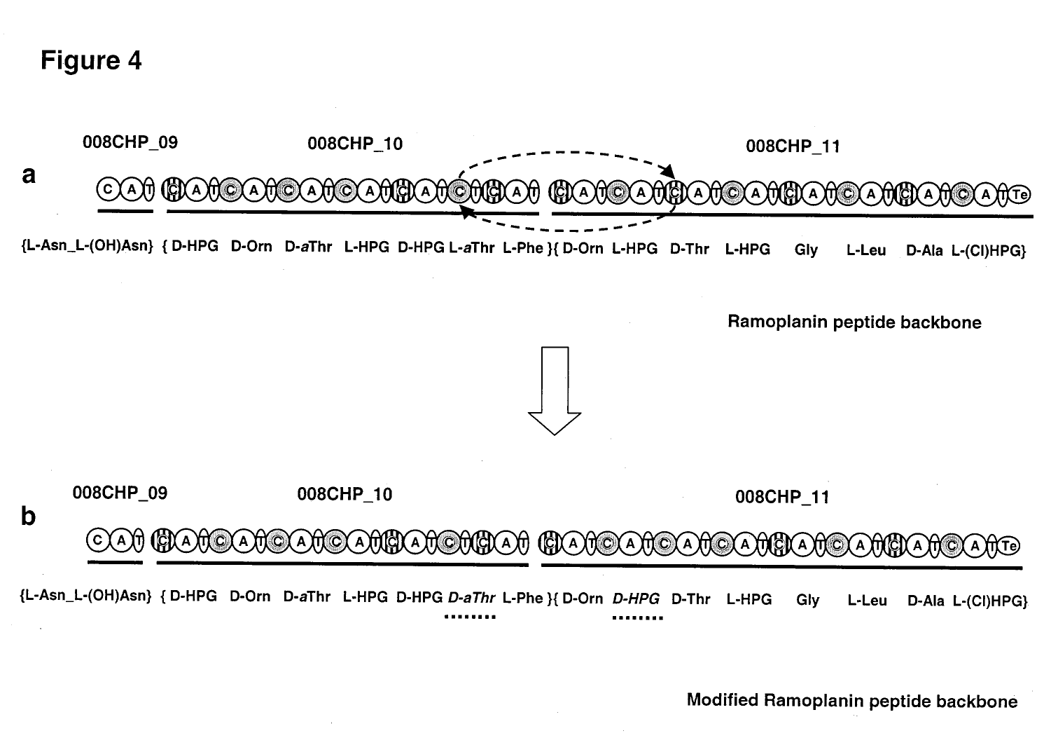

Ramoplanin is composed of 17 amino acid residues out of which 8 amino acid residues are D-enantiomers (Ciabatti et al.(1989), J. Antibiotics, 42, 254-267). Analysis of the RAMO locus revealed the presence of an NRPS system composed of 4 ORFs specifying a total of 16 modules involved in amino acid activation and condensation resulting in the synthesis of the ramoplanin peptide backbone (FIGS. 1c and 4a). Iterative use of ORF 008CHP_{—}09 insures N-acylation and condensation of the first two amino acid residues, namely L-asparagine (L-Asn) and L-hydroxyasparagine (L-(OH)Asn). ORF 008CKP_{—}04, composed of an adenylation domain fused to a thiolation domain, activates L-alloThreonine (L-aThr) and interacts in trans with the sixth module of 088CHP_{—}04. All NRPS modules are exclusively composed of a condensation-adenylation-thiolation tripartite module (C-A-T) that represents the minimal domain organization found in NRPS systems.

Determination of the stereochemistry of all amino acid residues found in the ramoplanin molecule implies the presence of epimerization domains (E-domains) in modules 1, 2, 3 and 5 of ORF 008CHP_{—}10 as well as in modules 1, 3 and 7 of ORF 008CHP_{—}11 that incorporate amino acid residues having a D-stereochemistry in the ramoplanin compound. However, the obvious lack of E-domains in the RAMO NRPS system suggests that alternative mechanisms for the generation and incorporation of D-amino acids may occur in the biosynthesis of ramoplanin. Sequence similarities between condensation (C-domains) and epimerization domains (Marahiel et al. (1997), Chem. Rev., 97, 2651-2673) prompted a detailed comparative analysis of all condensation domains present in the ramoplanin NRPS system.

The RAMO C-domains were compared to a collection of condensation domains derived from various peptide NRPSs obtained from GenBank or disclosed herein. FIG. 2 shows the evolutionary relatedness of all RAMO C- domains that clearly indicates the presence of three distinct classes of condensation domains. The first class comprises the unique acyl-specific C-domain that is found in ORF 008CHP-09 (FIG. 2, empty circle). This domain catalyzes the condensation of an acyl unit to the first amino acid incorporated in ramoplanin (L-Asn) as well as the condensation of the second amino acid residue, L-(OH)-Asn, found in the ramoplanin molecule. This type of condensation domain clusters with related domains found in lipopeptide-specifying NRPS systems and is described in detail in co-pending US application U.S. Ser. No. 10/329,027. A second class of C-domains is defined by domains that follow modules incorporating L-amino acids (FIG. 2, hatched circles). Condensation domains found in modules 1, 5 and 7 of ORF 008CHP_{—}10 as well as in modules 1, 3, 5 and 7 of ORF 008CHP_{—}11 belong to this class.

Surprisingly, a third class of C-domains composed of domains that follow modules incorporating D- or non-chiral amino acids is also present in the ramoplanin NRPS complex (FIG. 2, gray circles). These domains are found in modules 2, 3, 4 and 6 of ORF 008CHP_{—}10 (SEQ ID NOS: 01, 03, 05 and 07, respectively) as well as in modules 2, 4, 6 and 8 of ORF 008CHP_{—}11 (SEQ ID NOS: 09, 11, 13 and 15, respectively). The relative location of these domains following thiolation domains of modules that incorporate D-amino acids is reminiscent of that of epimerization domains. Clustering of these C-domains further supports the idea that they may have a dual function and catalyze both epimerization and condensation reactions. Indeed, these dual condensation/epimerization NRPS domains would be involved in the epimerization reaction, from L- to D-stereochemistry, of the amino acid activated by the preceding module and the condensation of this amino acid to the one specified by the cognate module. The location of all dual condensation/epimerization NRPS domains in the ramoplanin NRPS system concords in all cases with the expected location of D- or non-chiral amino acids in the ramoplanin molecule (FIG. 1c).

### EXAMPLE 2

### Presence of Dual Condensation/Epimerization NRPS Domains in the Syringomycin NRPS System

Syringomycin, produced by phytopathogenic strains of Pseudomonas syringae pv. syringae, is a cyclic lipodepsipeptide that exhibits phytotoxic activity and a wide spectrum of antimicrobial and antifungal properties (Bender et al. (1999), Microbiol. Mol. Biol. Rev., 63, 266-292). The syringomycin non-ribosomal peptide synthetase biosynthetic gene cluster of P. syringae pv. syringae strain B301D was sequenced and characterized (Guenzi et al. (1998), J. Biol. Chem., 273, 32857-32683). This led to the unexpected finding that the syringomycin NRPS system did not contain any epimerization domains. Syringomycin contains two D-amino acids (D-Ser in position 2 and D-2,4-diaminobutyric acid in position 3) and, thus, the authors were expecting to find epimerization domains in the corresponding modules 2 and 3 of the syringomycin NRPS complex. These same authors ruled out the possibility that the adenylation domain of module 2 might specifically recognize and activate D-Ser. Consequently, the means by which D-amino acids were incorporated into syringomycin remained a mystery.

Analysis of C-domains present in the syringomycin NRPS system confirms that 4 out 9 domains belong to the class of dual condensation/epimerization NRPS domains (FIGS. 1c and 2). The relative location of these dual condensation/epimerization NRPS domains is in agreement with the stereochemistry of the amino acid that is activated by the preceding module. For example, dual condensation/epimerization NRPS domain of SEQ ID NO: 19 in module 3 of ORF AAC80285 is located downstream of module 2 that incorporates D-Serine (D-Ser); dual condensation/epimerization NRPS domain of SEQ ID NO: 21 in module 4 of ORF AAC80285 is located downstream of module 3 that incorporates D-2,4-diaminobutyric acid (D_DAB); dual condensation/epimerization NRPS domain of SEQ ID NO: 23 in module 8 of ORF AAC80285 is located downstream of module 7 that incorporates non-chiral 2,3-dehydroaminobutyric acid (DHAB).

It is noteworthy that the C domain from module 2 of the syringomycin synthetase clusters among the dual condensation/epimerization NRPS domains, predicting that the amino acid incorporated by the upstream module is in the D- configuration. This is inconsistent with the reported stereochemistry of the amino acid in position 1 of syringomycin, L-Ser (Fukuchi et al. (1992), J. Chem. Soc. Perkin, 1, 1149-1157). One possible explanation for this discrepancy is that the assessment of the stereochemistry for the amino acid in position 1 of syringomycin is incorrect. Alternatively, this discrepancy may have arisen from the fact that different P. syringae pv. syringae strains were used for structure determination (strain SC1) and for sequencing of the biosynthetic cluster (strain B301 D). As the structure of the syringomycin produced by strain B301 D has not been confirmed to contain an L-Ser in position 1, it is possible that this strain produces a syringomycin that is distinct from that produced by strain SC1. Alternatively, a free-standing racemase that is able to catalyze epimerization of amino acids could be involved in the conversion of D-Ser to the L- enantiomer. This mechanism occurs in Microcystis aeruginosa, where the racemase McyF is involved in the conversion of L- to D-Glutamate in microcystin biosynthesis (Nishizawa et al. (2001), Microbiology, 147, 1235-1241). Yet another possibility is that, although the C domain in module 2 of the syringomycin synthetase displays significant overall homology to the specialized dual condensation/epimerization NRPS domains of E-less NRPSs, it may have acquired fine mutations that inactivate the epimerization function. If this is true, it may be possible to delineate the residues or motifs that confer upon these specialized C domains the ability to carry out a dual function.

### EXAMPLE 3

### Presence of Condensation/Epimerization Dual Domains in the Syringopeptin NRPS System

In silico sequence comparison of dual condensation/epimerization NRPS domains specified by the ramoplanin NRPS system with protein sequences contained in the GenBank protein database revealed sequence similarities between these domains and condensation domains from the syringopeptin NRPS system. Syringopeptins are lipodepsipeptide phytotoxins and are produced by strains of P.syringae pv. Syringae. The syringopeptin cluster present in strain B301D has been sequenced and encodes 22 NRPS modules involved in syringopeptin peptide synthesis (Scholz-Schroeder et al. (2003), Mol. Plant Microbe Interact, 16, 271-280).

Analysis of a clustal sequence alignment of C-domains from the syringopeptin NRPS cluster shows that all C-domains, with the exception of C-domain in module 1 of ORF AAF99707, and C-domains in modules 10, 11 and 12 in ORF AAO72424, belong to the class of dual condensation/epimerization NRPS domains (FIGS. 1d and 2). This observation leads to the prediction that amino acid residues 1 to 18 are in the D-specific configuration whereas amino acid residues 19 to 22 are in the L-form (FIG. 2). Experimental analysis of the stereochemistry of the amino acid components of syringopeptin 22-A confirms that 17 out of 22 amino acids are in the D-configuration (Ballio et aL (1995), Eur. J. Biochem., 234, 747-758). Examination of the relative position of C/E dual domains confirms the prediction of stereochemistry for the syringopeptin amino acid components as all NRPS modules preceding a dual condensation/epimerization NRPS domain incorporate D-amino acids with the exception of three modules: module 4 of ORF AAF99707 incorporates L-Valine (L-Val) instead of the predicted D-Val residue; module 2 of ORF AAO72424 incorporates L-Alanine (L-Ala) instead of the predicted D-Ala amino acid component and module 11 of ORF AAO72424 incorporates D-2,4-diaminobutyric acid (D-DAB) instead of the predicted L-form of the amino acid (FIG. 1d). Observed discrepancies may be due to incorrect assignments of stereochemistry and/or possible presence of mutations in the dual condensation/epimerization NRPS domains inactivating the epimerization function of the domains. Alternatively, free-standing racemases could modify the stereochemistry of specific amino acids and thus contribute to the observed differences.

### EXAMPLE 4

### Detection of Dual Condensation/Epimerization NRPS Domains NRPS Systems Present in Diverse Microorganisms

In silico sequence analysis of dual condensation/epimerization NRPS domains specified by the ramoplanin NRPS system with protein sequences contained in the GenBank database revealed sequence similarities between these domains and C-domains from an unknown NRPS gene cluster present in Ralstonia solanacearum strain GMI1000 (Salanoubat et al. (2002), Nature, 415, 497-502) referred to herein as URSO (FIG. 1d). Clustal sequence alignment of C-domains specified by the URSO gene cluster indicates the presence of two C/E dual domains in modules 1 and 3 of NRPS ORF CAD17793 (FIG. 2). Based on the relative location of the two dual condensation/epimerization NRPS domains, the peptide encoded by URSO is predicted to contain D-amino acid components (D-aa4 and D-aa6) incorporated by NRPS modules 4 and 6 (FIG. 1d).

Using the same approach, the proprietary Ecopia BioSciences DECIPHER® database that is populated with a variety of gene clusters involved in microbial secondary metabolism was assessed for the presence of NRPS gene clusters containing dual condensation/epimerization NRPS domains. In this way, several NRPS clusters were detected from a great variety of microorganisms. Representative examples include the following gene clusters.

Unreported NRPS cluster found in Streptomyces aizunensis strain NRRL B11277: Locus 023C is predicted to encode a lipopeptide as it is composed of a NRPS multienzymatic system starting with an N-acyl-specific condensation domain that was previously shown to condense acyl groups to the amino group of amino acids (U.S. Ser. No. 10/329,027; FIG. 2). This gene cluster contains 28 NRPS modules all composed of minimal tripartite modules (C-A-T). The sequence of the first component of the 023C. NRPS complex is broken into two portions, an N-terminal fragment (023CSPF09) and a C-terminal fragment (023CSPF10) due to an apparent frameshift in the region corresponding to the first A domain. ORF 023CYP_{—}11 corresponds to a free-standing adenylation domain that interacts in trans with module 21 located in ORF 023CYP_{—}01 of the NRPS system (FIG. 1e). Clustal sequence alignment analysis of C-domains indicates the presence of 14 C/E dual domains that are predicted to epimerize and condense amino acid components D-aa1, D-aa2, D-aa3, D-aa6, D-aa7, D-aa8, D-aa10, D-aa11, D-aa15, D-aa21, D-aa22, D-aa24, D-aa25 and D-aa26 (FIGS. 1e and 2).

Unknown NRPS cluster found in Streptomyces griseofuscus strain NRRL B-5429: Locus 034F is predicted to encode a lipopeptide as it is composed of a NRPS multienzymatic system starting with an N-acyl-specific condensation domain that condenses acyl groups to the amino group of amino acids (FIGS. 1f and 2)). This gene cluster contains 10 NRPS modules composed of minimal tripartite modules (C-A-T) and one module present in ORF 034CMP_{—}78 that contains an epimerization domain (FIG. 1f). Clustal sequence alignment analysis of C-domains indicates the presence of four dual condensation/epimerization NRPS domains that are predicted to epimerize and condense amino acid components D-aa5, D-aa6, D-aa8 and D-aa10 and the presence of a C-domain that belongs to the class of C-domains found downstream of epimerization domains (FIGS. 1f and 2).

Unknown NRPS cluster found in Kitasatosporia sp. strain ECO-03: 040G is a partial gene cluster contained in an actinomycetes strain present in the Ecopia culture collection and predicted to encode a lipopeptide as judged by the presence of an acyl-specific condensation domain in the starter module of the NRPS system (FIGS. 1f and 2). The incomplete gene cluster contains at least nine NRPS modules all composed of minimal tripartite modules (C-A-T). Clustal sequence alignment analysis of C-domains indicates the presence of two dual condensation/epimerization NRPS domains that are predicted to epimerize and condense amino acid components D-aa2 and D-aa6 (FIGS. 1f and 2).

Unknown NRPS cluster found in Streptomyces sp. strain ECO-38: 084B is a gene cluster contained in an actinomycetes strain present in the Ecopia culture collection and predicted to encode a lipopeptide as judged by the presence of an acyl-specific condensation domain in the starter module of the NRPS system (FIGS. 1g and 2). This gene cluster contains nine NRPS modules all composed of minimal tripartite modules (C-A-T). The sequence of the first component of the 084B NRPS complex is broken into two portions, an N-terminal fragment (084CBP_{—}46) and a C-terminal fragment (084CBP_{—}47) due to an apparent frameshift in the region corresponding to the second C domain (FIG. 1g). Clustal sequence alignment analysis of C-domains indicates the presence of four dual condensation/epimerization NRPS domains that are predicted to epimerize and condense amino acid components D-aa3, D-aa4, D-aa5 and D-aa6 (FIGS 1g and 2).

Unknown NRPS cluster found in Streptomyces sp. strain ECO-59: 107A is a partial gene cluster contained in an actinomycetes strain present in the Ecopia culture collection and predicted to encode a lipopeptide as judged by the presence of an acyl-specific condensation domain in the starter module of the NRPS system (FIGS. 1g and 2). The incomplete gene cluster contains so far 5 NRPS modules all composed of minimal tripartite modules (C-A-T). The sequence of the first component of the 107A NRPS complex is broken into three portions: an N-terminal fragment (107CAP_{—}08), a middle fragment (107CAPC02) and a C-terminal fragment (107CAP_{—}12) due to two small sequencing gaps of approximately 100 basepairs or less in the region corresponding to the C-A junctions in modules 1 and 2 (FIG. 1g). Clustal sequence alignment analysis of C-domains indicates the presence of two dual condensation/epimerization NRPS domains that are predicted to epimerize and condense amino acid components D-aa1 and D-aa2 (FIGS. 1g and 2).

Unknown NRPS cluster found in Streotomyces viridifaciens strain NRRL ISP-5239: Locus 143F is predicted to encode a lipopeptide as it is composed of a NRPS multienzymatic system starting with an N-acyl-specific condensation domain that condenses acyl groups to the amino group of amino acids (FIGS. 1h and 2). This gene cluster contains ten NRPS modules all composed of minimal tripartite modules (C-A-T) (FIG. 1h). Clustal sequence alignment analysis of all C-domains indicates the presence of four dual condensation/epimerization NRPS domains that are predicted to epimerize and condense amino acid components D-aa1, D-aa5, D-aa8 and D-aa9 (FIGS. 1h and 2).

Unknown NRPS cluster found in Actinomadura sp. strain ATCC. 39334: Locus 153A is predicted to encode a lipopeptide as it is composed of a NRPS multienzymatic system starting with an N-acyl-specific condensation domain that was previously shown to condense acyl groups to the amino group of amino acids (U.S. Ser. No. 10/329,027; FIGS. 1h and 2). This gene cluster contains sixteen NRPS modules all composed of minimal tripartite modules (C-A-T) (FIG. 1h). Clustal sequence alignment analysis of C-domains indicates the presence of four dual condensation/epimerization NRPS domains that are predicted to epimerize and condense amino acid components D-aa4, D-aa12, D-aa13 and D-aa14 (FIGS. 1h and 2).

Ramoplanin-like NRPS cluster found in Micromonospora chersina strain ATCC. 53710: Locus 263B is a partially characterized NRPS locus that is predicted to encode a ramoplanin-like lipopeptide. Analysis of the specificities of the adenylation domains (Challis and Ravel, supra) present in the NRPS complex indicates that the nature and relative order of the amino acid components specified by 263B are identical to those of ramoplanin (FIG. 1i). The incomplete gene cluster contains so far five NRPS modules all composed of minimal tripartite modules (C-A-T) and a partial module comprising a C-domain. The sequence of the second component of the 263B NRPS complex is broken into two portions, an N-terminal fragment (263CRPN44) and a C-terminal fragment (263CRPN18) due to a small sequencing gap of approximately 100 basepairs or less in the region corresponding to the C-A junctions in modules 1 and 2 (FIG. 1i). Clustal sequence alignment analysis of C-domains indicates the presence of four dual condensation/epimerization NRPS domains that are predicted to epimerize and condense amino acid components D-Asparagine (D-Asn), D-Hydroxyphenylglycine (D-HPG), D-Ornithine (D-Orn) and D-Threonine (D-Thr) (FIGS. 1i and 2). Even though the respective order and nature of the amino acid components of 263B are predicted to be identical to those determined in ramoplanin, the stereochemistry of the compound is predicted to be different as module 1 of the 263B NRPS complex that activates asparagine precedes a dual condensation/epimerization NRPS domain that will epimerize and condense this amino acid residue to its cognate amino acid residue, L-Hydroxyasparagine (FIG. 1i).

### EXAMPLE 5

### Exchange of a Dual Condensation/Epimerization NRPS Domain and a Regular Condensation Domain in the Ramoplanin NRPS System Alters the Strereochemistry of Ramoplanin at two Different Amino Acid Components

The availability of dual condensation/epimerization NRPS domains increases the potential of redesigning (un)natural products by engineered peptide synthetases. Functional hybride peptide synthetases may be engineered to producing rationally designed peptide products using known molecular biological techniques (see, for example, Mootz et al. (2000), Proc. Natl. Acad. Sci. USA., 97, 5848-5853). Domain swapping, change-of-substrate specificity by mutagenesis, and induced termination to achieve release of a defined shortened product are used to generate a recombinant NRPS system producing antipain, a potent cathepsin inhibitor produced by Streptomyces roseus and whose biosynthetic machinery is unknown (Doekel and Marahiel (2001), Metab. Eng., 3, 64-77). Mootz et al., supra, describes genetic engineering using an NRPS system to produce a peptide product that is not a naturally occurring product, and Doekel and Marahiel supra describes engineering an NRPS system to make the known natural product antipain.

The NRPS biosynthetic machinery of peptide natural product ramoplanin can be modified so as to produce modified versions of ramoplanin having altered stereochemistries.

Domain organization of the NRPS complex involved in ramoplanin biosynthesis is described in Example 1. Eight dual condensation/epimerization NRPS domains catalyzing the condensation of D-amino acid residues and eight regular C-domains that condense L- amino acid residues are present (FIG. 4a, panel a). Through domain swapping, a regular C-domain is replaced by a dual condensation/epimerization NRPS domain. Preferably, the dual condensation/epimerization NRPS domain located in module 6 of ORF 008CHP_{—}10 involved in epimerization and condensation of L-hydroxyphenylglycine (L-HPG) to L-threonine (L-Thr) is used to replace the C-domain in module 3 of ORF 008CHP_{—}11 as the former domain is naturally specific for condensing L-HPG and L-Thr. The modified ramoplanin locus contains a dual condensation/epimerization NRPS domain in module 3 of ORF 008CHP_{—}11 resulting in a change of stereochemistry of the preceding amino acid residue from L- to D-HPG (FIG. 4a, panel b). Additional changes in the stereochemistry of the modified compound may be accomplished by further exchanging additional C-domains and dual condensation/epimerization NRPS domains from various NRPS systems.

The recombinant NRPS system depicted in FIG. 4a is employed either in vivo, using an appropriate recombinant host, or in vitro, using purified enzymes supplemented with the appropriate substrates. Stereochemically modified ramoplanin analogues are generated in vivo using of Actinoplanes sp. ATCC. 33076, the ramoplanin producer, as the host strain. The C-domain is physically replaced by double recombination (Kieser et al. (2000), Practical Streptomyces Genetics (The John Innes Foundation, Norwich, UK)). Stereochemically modified ramoplanin analogues are generated in vitro using over-expression of the recombinant 008CHP_{—}10 and 008CHP_{—}11 polypeptides in an appropriate host, for example E. coli, followed by the preparation of an extract or purified fraction thereof and use of said preparation together with appropriate substrates as outlined in Mootz et al. supra. In the absence of accessory proteins the product produced by this in vitro system is not expected to contain modifications such as N-acylation or glycosylation present in the natural ramoplanin molecule.

In this example, the use of dual condensation/epimerization NRPS domains for modifying the stereochemistry of peptide compounds presents major advantages over insertion of canonical epimerization domains in specific NRPS locations. Insertion of foreign domains in the NRPS complex may interfere with the activities of existing domains. For example, Schauwecker et al. (2000), Chem. Biol., 7, 287-297 demonstrated that replacement of an adenylation domain by an adenylation-methylation domain that is twice as long as the replaced domain, interferes with the activity of the cognate epimerization domain present in the specific module. However, use of dual condensation/epimerization NRPS domains instead of epimerization domains to specifically alter the stereochemistry of a peptide compound is expected to alleviate this problem as both regular condensation domains and dual condensation/epimerization NRPS domains are structurally similar.

### EXAMPLE 6

### Dual Condensation/Epimerization NRPS Domain From the Ramoplanin NRPS System is Used to Modify the Stereochemistry of the Complestatin Molecule at a Specific Amino Acid Component

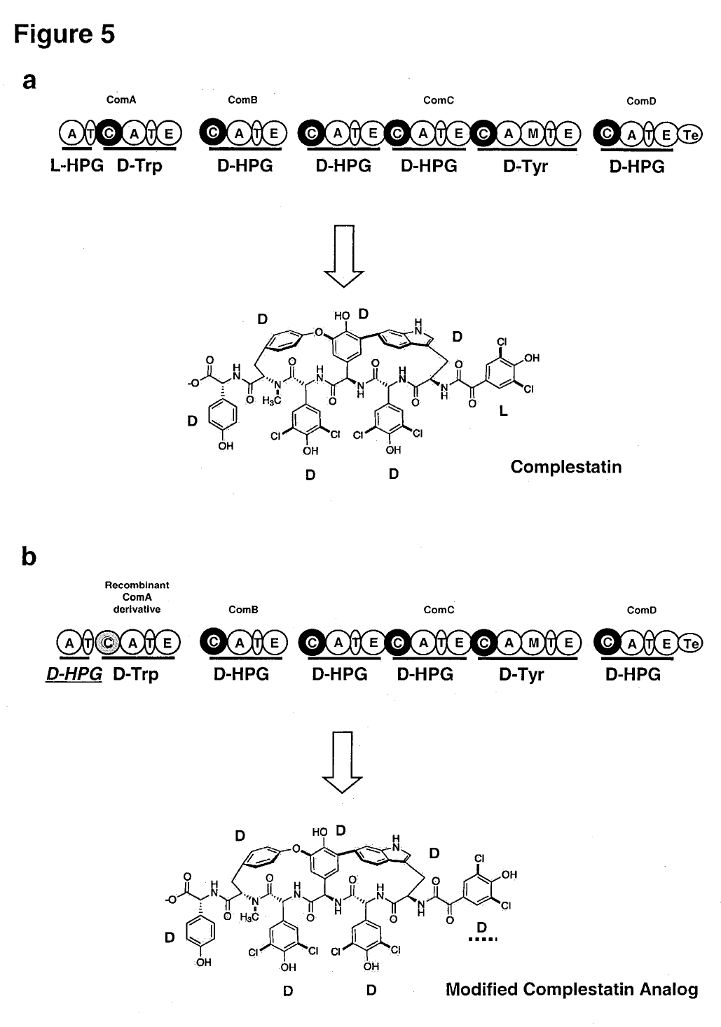

The NRPS biosynthetic machinery of peptide natural product complestatin can be modified so as to produce modified versions of complestatin having altered stereochemistries, as shown diagrammatically in FIG. 5. Complestatin, a member of the vancomycin group of natural products produced by Streptomyces lavendulae, consists of an alpha-ketoacyl hexapeptide backbone modified by oxidative phenolic couplings and halogenations. The entire complestatin biosynthetic and regulatory gene cluster spanning ca. 50 kb was cloned and sequenced (Chiu et al. (2001), Proc. Natl. Acad. Sci. USA, 98, 8548-8553). It includes four NRPS genes, comA, comB, comC, and comD (FIG. 4b, panel a). The comA gene encodes an NRPS that is composed of a loading module (A-T) and a complete module (C-A-T-E) containing an epimerization domain. Module 2 catalyzes conversion of L- to D-Tryptophan (Trp) and condensation to the preceding amino acid, L-hydroxyphenylglycine (L-HPG). Complestatin is almost entirely composed of D-amino acids with the exception of the first L-HPG residue. The stereochemistry of complestatin is altered by domain swapping between the C-domain of ComA and a dual condensation/epimerization NRPS domain. The dual condensation/epimerization NRPS domain located in module 6 of ORF 008CHP_{—}10 involved in epimerization of L-hydroxyphenylglycine (L-HPG) and condensation of this amino acid to L-threonine (L-Thr) (FIG. 4a, panel a) is used to replace the C-domain of ComA, as the former domain is naturally specific for condensing L-HPG found at this position in the complestatin structure. This modification results in a complestatin derivative having an amino terminal amino acid in the D- configuration (D-HPG) (FIG. 4b, panel b). Additional changes in the stereochemistry of the modified compound may be accomplished by further exchanging additional C-domains and dual condensation/epimerization NRPS domains from various NRPS systems.

The recombinant NRPS system depicted in FIG. 4b is employed either in vivo, using an appropriate recombinant host or in vitro using purified enzymes supplemented with the appropriate substrates. Stereochemically modified complestatin analogues are generated in vivo using Streptomyces lavendulae, the complestatin producer, as the host strain. This is accomplished by physically replacing C-domains by way of double recombination (Keiser et aL, supra). Stereochemically modified complestatin analogues are generated in vitro by over-expression of the recombinant ComA, ComB, ComC. and ComD polypeptides in an appropriate host, for example E. coli, followed by the preparation of an extract or purified fraction thereof and use of said preparation together with appropriate substrates as outlined in Mootz et al. supra. In the absence of accessory proteins the product produced by this in vitro system are not expected to contain modifications such as the cross-linking of residues that is catalyzed by specific complestatin cytochrome P450 enzymes.

In this example, the use of dual condensation/epimerization NRPS domains for modifying the stereochemistry of peptide compounds presents major advantages over insertion of canonical epimerization domains in specific NRPS locations. Insertion of foreign domains in the NRPS complex may interfere with the activities of existing domains. For example, Schauwecker et al. (2000), Chem. Biol., 7, 287-297 demonstrated that replacement of an adenylation domain by an adenylation-methylation domain that is twice as long as the replaced domain, interferes with the activity of the cognate epimerization domain present in the specific module. However, use of dual condensation/epimerization NRPS domains instead of epimerization domains to specifically alter the stereochemistry of a peptide compound is expected to alleviate this problem as both regular condensation domains and dual condensation/epimerization NRPS domains are structurally similar.

Although the present invention has been described hereinabove by way of preferred embodiments thereof, it can be modified without departing from the spirit, scope and nature of the subject invention, as defined in the appended claims. All of the published patent applications and issued patents mentioned in this application are hereby incorporated by reference.

What is claimed is:

1. An isolated polynucleotide encoding a dual condensation/epimerization NRPS domain, wherein said polynucleotide encodes a polypeptide having at least 45% sequence identity to SEQ ID NO: 139.

2. An isolated polynucleotide as defined in claim 1 comprising a sequence selected from the group consisting of:
(a) a sequence selected from the group consisting of SEQ ID NOS: 2, 4, 6, 8,10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136 and 138;
(b) a sequence that is complementary to (a);
(c) a sequence which hybridizes to said sequence of (a) or (b) under conditions of high stringency; and
(d) a sequence which has at least 60% or higher identity to said sequence of (a), (b), or (c) as measured with BLASTN version 2.0 using the default parameters.

3. An isolated polynucleotide as defined in claim 2 comprising a sequence selected from the group consisting of:
(a) a sequence selected from the group consisting of SEQ ID NOS: 2, 4, 6, 8,10, 12, 14, 16, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136 and 138;
(b) a sequence that is complementary to (a);
(c) a sequence which hybridizes to said sequence of (a) or (b) under conditions of high stringency; and
(d) a sequence which has at least 60% or higher identity to said sequence of (a), (b), or (c) as measured with BLASTN version 2.0 using the default parameters.

4. An isolated polynucleotide as defined in claim 1 which is derived from an organism of the actinomycetes taxon.

5. An isolated polynucleotide as defined in claim 1, wherein said dual condensation/epimerization NRPS domain resides in a gene locus selected from the group consisting of:
RAMO, SYRI, SYRP, URSO, 023C, 034F, 040G, 084B, 107A, 143F, 153A and 263B.

6. An isolated polynucleotide as defined in claim 5, wherein said dual condensation/epimerization NRPS domain resides in a gene locus selected from the group consisting of:
RAMO, SYRI, SYRP and URSO.

7. An isolated polynucleotide as defined in claim 5, wherein said dual condensation/epimerization NRPS domain resides in a gene locus selected from the group consisting of:
023C, 034F, 040G, 084B, 107A, 143F, 153A and 263B.

8. An isolated polynucleotide as defined in claim 7, wherein said dual condensation/epimerization NRPS domain resides in a gene locus selected from the group consisting of:
023C, 034F, 143F, 153A and 263B.

9. An isolated polynucleotide as defined in claim 1 encoding a polypeptide sequence selected from the group consisting of:
(a) a sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135 and 137; and
(b) a sequence which has at least 60% or higher sequence identity or similarity to said sequence of (a) as determined using the BLASTP 2.2.5 algorithm.

10. An isolated polynucleotide as defined in claim 9 encoding a polypeptide sequence selected from the group consisting of:
(a) a sequence selected from the group consisting of SEQ ID NOS: 1, 3, 5, 7, 9, 11, 13, 15, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135 and 137; and
(b) a sequence which has at least 60% or higher sequence identity or similarity to said sequence of (a) as determined using the BLASTP 2.2.5 algorithm.

11. An isolated polynucleotide encoding a dual condensation/epimerization NRPS domain that produces an alignment with at least 45% identity with the following consensus sequence using the BLASTP 2.2.5 algorithm, with the filter option -F set to false, the gap opening penalty-G set to 11, the gap extension penalty -E set to 1, and all remaining options set to default values:
ADIYPLAPLQEGILFHHLladggedDaYVIpavIeFDSReRLdaFIgALQ qViDRHDlLRTavvWeGLrEPVQvvwRhAeLpVeevtLdpagiaadpvaq LdaaaglrmDLgrAPLlrlhvAadpgggrWLaLLrfHHLVqDHTALevLI aEiqAfLaGrgdeLPePIPFRnFVAQARIGvsraEHErFFaeLLGDVtEP TAPFGLIDVrGDGsgveearlpldaeLaaRLReqARrLGVSpATlfHLAW
ARVLgavSGRdDvvFGTVLfGRMqaGaGADRvpGIFINTLPVRVrIggqg VIdAVramRaqLAeLLeHEHAPLALAQRASGVpaptPLFTsLLNYRHsav aavsaealaawagaeleGirlLssrERTNYPLtVsVDDIGdgFsLtVqAv aplDaerVcallhTAlenLVdALEqaPdtpLsavdVLpaaERrrlLveWN dtaadyvpaatvpeLFeAQVartP.

12. An isolated polynucleotide as defined in claim 1 wherein said dual condensation/epimerization NRPS domain is involved in the incorporation of a D-amino acid or non-chiral amino acid into a peptide product.

13. An expression vector comprising an isolated polynucleotide as defined in claim 1 operably linked to an expression control sequence.

14. A cultured cell comprising a vector as defined in claim 13.

15. A cultured cell comprising an isolated polynucleotide as defined in claim 1 operably linked to an expression control sequence.

16. A cultured cell transfected with a vector as defined in claim 13, or a progeny of said cell, wherein the cell expresses a dual condensation/epimerization NRPS domain.

17. A cultured cell as defined in claim 16 selected from the group consisting of Actinoplanes sp. ATCC. 33076 and Streptomyces lavendulae.

18. A method for producing a dual condensation/epimerization NRPS domain, the method comprising culturing a cell as defined in claim 16 under conditions permitting expression of the dual condensation/epimerization NRPS domain.

19. A method for producing a dual condensation/epimerization NRPS domain, the method comprising culturing a cell as defined in claim 16 under conditions permitting expression under the control of the expression control sequence, and purifying the dual condensation/epimerization NRPS domain from the cell or the medium of the cell.

20. A method for epimerization and condensation of amino acids in E-less NRPS systems which comprises use of a dual condensation/epimerization NRPS domain encoded by an isolated polynucleotide as defined in claim 1.

21. A method for modifying the stereochemistry of a synthesized peptide compound in vivo in an appropriate recombinant host which comprises use of a dual condensation/epimerization NRPS domain encoded by an isolated polynucleotide as defined in claim 1.

22. A method as defined in claim 21, wherein the host is selected from the group consisting of Actinoplanes sp. ATCC. 33076 and Streptomyces lavendulae.

23. A method for modifying the stereochemistry of a synthesized peptide compound in vitro which comprises use of a dual condensation/epimerization NRPS domain encoded by an isolated polynucleotide as defined in claim 1.

24. A method as defined in claim 21, wherein a dual condensation/epimerization NRPS domain is genetically substituted for a regular condensation domain.

25. A method as defined in claim 21 to modify the strereochemistry of ramoplanin.

26. A method as defined in claim 21 to modify the stereochemistry of the complestatin molecule at a specific amino acid component.

27. A method for incorporating a D-amino acid into a peptide product which comprises use of a dual condensation/epimerization NRPS domain encoded by an isolated polynucleotide as defined in claim 1.

28. An isolated dual condensation/epimerization NRPS domain having at least 45% sequence identity to SEQ ID NO: 139.

29. An isolated domain as defined in claim 28 comprising a sequence selected from the group consisting of:
(a) a sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135 and 137;
(b) a sequence that is complementary to (a);
(c) a sequence which hybridizes to said sequence of (a) or (b) under conditions of high stringency; and
(d) a sequence which has at least 60% or higher identity or similarity to said sequence of (a), (b), or (c) as measured with BLASTP version 2.2.5 using the default parameters.

30. An isolated domain as defined in claim 29 comprising a sequence selected from the group consisting of:
(a) a sequence selected from the group consisting of SEQ ID NOS: 1, 3, 5, 7, 9, 11, 13, 15, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135 and 137;
(b) a sequence that is complementary to (a);
(c) a sequence which hybridizes to said sequence of (a) or (b) under conditions of high stringency; and
(d) a sequence which has at least 60% or higher identity or similarity to said sequence of (a), (b), or (c) as measured with BLASTP version 2.2.5 using the default parameters.

31. An isolated domain as defined in claim 28 comprising a sequence selected from the group consisting of:
(a) a sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135 and 137;
(b) a sequence that is complementary to (a);
(c) a sequence which hybridizes to said sequence of (a) or (b) under conditions of high stringency; and
(d) a sequence which has at least 70% or higher identity or similarity to said sequence of (a), (b), or (c) as measured with BLASTP version 2.2.5 using the default parameters.

32. An isolated domain as defined in claim 31 comprising a sequence selected from the group consisting of:
(a) a sequence selected from the group consisting of SEQ ID NOS: 1, 3, 5, 7, 9, 11, 13, 15, 65, 67, 69, 71, 73, 75, 77, 79, 81, 83, 85, 87, 89, 91, 93, 95, 97, 99, 101, 103, 105, 107, 109, 111, 113, 115, 117,119, 121, 123, 125, 127, 129, 131, 133, 135 and 137;
(b) a sequence that is complementary to (a);
(c) a sequence which hybridizes to said sequence of (a) or (b) under conditions of high stringency; and
(d) a sequence which has at least 70% or higher identity or similarity to said sequence of (a), (b), or (c) as measured with BLASTP version 2.2.5 using the default parameters.

33. An isolated domain as defined in claim 28, wherein said domain is involved in the incorporation of a D-amino acid or non-chiral amino acid into a peptide product.

34. A dual condensation/epimerization NRPS domain that produces an alignment with at least 45% identity with the following consensus sequence using the BLASTP 2.2.5 algorithm, with the filter option -F set to false, the gap opening penalty -G set to 11, the gap extension penalty -E set to 1, and all remaining options set to default values:
ADIYPLAPLQEGILFHHLIadggedDaYVIpavIeFDSReRLdaFIgALQ qViDRHDlLRTavvWeGLrEPVQvvwRhAeLpVeevtLdpagiaadpvaq LdaaagLrmDLgrAPLlrlhvAadpgggrWLaLLrfHHLVqDHTALevLI aEiqAfLaGrgdeLPePIPFRnFVAQARIGvsraEHErFFaeLLGDVtEP TAPFGLIDVrGDGsgveearIpIdaeLaaRLReqARrLGVSpATIfHLAW ARVLgavSGRdDvvFGTVLfGRMqaGaGADRvpGIFINTLPVRVrIggqg VIdAVramRaqLAeLLeHEHAPLALAQRASGVpaptPLFTsLLNYRHsav aavsaealaawagaeleGirlLssrERTNYPLtVsVDDIGdgFsLtVqAv apIDaerVcallhTAlenLVdALEqaPdtpLsavdVLpaaERrrlLveWN dtaadyvpaatvpeLFeAQVartP.

35. A dual condensation/epimerization domain contained in cosmid 008CH having accession number IDAC 190901-3 or cosmid 008CK having accession number IDAC 190901-1.


SEQUENCE LISTING (139 sequences)

SEQ ID NO: 1 - protein, 456 aa, Actinoplanes sp.
ADIYPLAPLQEGIFFHHLMTEGDTADVYALPYLLRVGTREQLDAFLGALQQVVDRHDVYRTAIAWQNLREPVQVVHRHATLPVTEVTPDQLHAAATGGRLPLDHAPLLSVHIAPEPDGGWLALLRMHHLVQDHTALDIVLDEIRTILAGATDHLPPPVPFRNFVARSRRGAAEAAHRDYFTGLLGDVTETTAPYGLTDVHGEHSGVRRGRLAVSAGLAGRVRETARDRGVSPATLFHLAWARVLAAVSGRDDVVFGTVLLGRMDAGPGADRVPGLFMNTLPVRVRLGGRTVDEALHGMRAQLADLLTHEHAPLVLAQQSAGLPGGSPLFTSLFNYRHNATDIERSGTGIDGVEALPTGDPSNYPLDVSVNQSPLGFELVVEATEPADPDQLCRLLHACLDDLIAALDEQPGRALGTLDVVAGRERDLLLDGWNATAVPAQPALVPELFTAQAARTP

SEQ ID NO: 2 - DNA, 1368 bp, Actinoplanes sp.
gccgacatctaccccctagccccgctgcaagagggcatcttcttccaccacctcatgaccgagggcgacaccgccgacgtctacgcgctgccgtacctgctgcgcgttggcacgcgtgagcagctcgacgccttcctcggggctttgcagcaggtggtggaccgccacgacgtctaccgcacggccatcgcctggcagaacctgcgcgagcccgtccaggtcgtgcaccgccacgccaccctgcccgtcaccgaagtcacccccgaccaactgcacgccgccgccaccggcggccggctcccgctcgaccacgcacccctgctcagcgtccacatcgcacccgaacccgacggcggctggctcgccctactgcgcatgcaccacctcgtgcaggaccacaccgccctcgacatcgtcctcgacgagatccgcaccatcctcgccggcgcaaccgaccacctccccccgcccgtaccgttccgcaatttcgtggcgcgctcgcggcgtggtgccgccgaggccgcgcaccgcgactacttcaccggcctgctcggcgacgtcaccgagaccaccgccccgtacggcctcaccgacgtgcacggtgagcactccggcgtgcgccggggccggctcgccgtgtccgccgggctcgccggccgggtgcgggagaccgctcgcgaccggggcgtcagccccgccaccctcttccacctggcctgggcgcgcgtgctcgccgccgtctcgggccgcgacgacgtcgtcttcggcaccgtgctgctgggccggatggatgccggcccgggcgccgaccgggtgccgggccttttcatgaacaccctgccggttcgcgtacgcctcggcggccgcaccgtcgacgaggcgctgcacgggatgcgcgcccagctcgccgacctgctcacccacgagcacgccccgctggtgctcgcgcagcagtcggccggcctgcccggcggcagccccctgttcacctcgctgttcaactaccggcacaacgcgaccgacatcgagcgctccggaacgggcatcgacggcgtcgaggcgctgcccaccggcgacccgtcgaactatccgctcgacgtctcggtcaaccagagcccgctcggcttcgagctcgtcgtcgaggccaccgagccggccgacccggaccagctctgccggctcctgcacgcctgcctcgacgacctgatcgccgccctcgacgagcagcccggccgcgcgctcggcaccctcgacgtcgtcgccggacgggagcgcgacctcctcctggacggctggaacgccacggcggtcccggcccagccggccctggtgccggagctcttcacggcgcaggcggcgcggacaccc

SEQ ID NO: 3 - protein, 451 aa, Actinoplanes sp.
ADVYPLAPLQEGIFFPVATGPQCYATVGSSLPDDGGSAPCSRFRRRCVSTSVVWQGLREPVQVVWRHARLPVEEVVLHEGADPVEQMMALAGGWMDLTRAPLIDVHIAAGPGGDRWLAVLRIHHLVQDHTALETLLDELQSFLEGRGGELAEPVPFREFVAQARLGVPREEHERYFAELLGDITETTAPYDLTDVHGDGTGYDHGALPLDATVAARVREAARTLGVSPATLFHLAWARVLGTLAGRDDVVFGTVLFGRMNSGAGADRVSGLFINTLPVRVRLGAPTGDALGDLRDQLAELLVHEHASLASAQKASGLPGGSPLFTSIFNYRHNQVSAERETAALPGIRVLAARDSTNYPLTVAVDDDGHGFTLVVEVASTVDAAGVCELLHTAVDNLIAALTDRPGGPLAEVDILERGLRDRLLTAWNEAREPAPPVTLPDLFDRQARRTP

SEQ ID NO: 4 - DNA, 1353 bp, Actinoplanes sp.
gccgatgtgtatccgttggcgccgttgcaggagggcatcttcttccccgtagccacaggcccccagtgctacgccacggtggggagttcactcccggacgatggcggttctgccccttgcagcaggtttcgccgtcgatgtgtatcgacgagtgtggtgtggcaggggctgcgtgagccggtgcaggtggtgtggcggcacgcgcggctgcccgtcgaggaggtcgtgctgcacgagggggccgacccggtcgagcagatgatggcgctcgccggcggttggatggacctcacccgggcgccgctcatcgacgtccacatcgccgccggccccggcggcgaccgctggctggccgtgctgcgcatccaccacctcgtgcaggaccacaccgccctggagacgctgctcgacgagctgcagtcctttctggagggccgcggtggcgagcttgccgagccggtgccgttccgcgagttcgtggcgcaggcgcggctcggtgtgccgcgcgaggagcacgagcggtatttcgcggagttgctcggcgacatcaccgagaccaccgcgccgtacgacctgaccgacgtgcacggcgacggcaccggatacgaccacggcgcgctgccgctggacgccaccgtcgcggcccgcgtccgggaggcggcccgaaccctcggcgtcagcccggcgacgctcttccacctcgcgtgggcgcgggtgctcggcacgctggccgggcgcgacgacgtcgtcttcggcaccgtcctgttcggacggatgaactcgggtgccggcgccgaccgggtctccggcctgttcatcaacacgctgccggtgcgggtgcggctcggcgcgcccaccggcgacgccctcggcgacctccgcgaccagctcgccgagctgctcgtgcacgagcacgcctccctcgcctccgcgcagaaggcgagcggcctgcccggcgggagccccctgttcacgtcgatcttcaactaccggcacaaccaggtgagcgccgaacgggagaccgccgcgctgcccggcatccgcgtcctcgcggcccgcgactccacgaactatccgctcaccgtcgcggtggacgacgacgggcacggcttcacgctcgtggtcgaggtcgcgtccacagtcgacgccgcgggcgtctgcgaactgctgcacaccgccgtggacaacctcatcgcggccctcaccgaccggccgggcgggccgctggccgaggtcgacatcctcgaacgcggtctgcgggaccgcctgctgaccgcctggaacgaggcccgggagcccgcaccgccggtgaccctgcccgacctgttcgaccggcaggcccgccgcacgccc

SEQ ID NO: 5 - protein, 461 aa, Actinoplanes sp.
ADIYPLAPLQEGIFFHHLMADAGRADAYAMPYVLHLDTAERLDVLLGALQRVIDRNDIYRTGVVSAGLREPVQVVWRSAVLPVEEVALDGGHDPVEQLLAAAGEEFDLTRAPLIRAHVAAHPDGGRLLLLRIHHLVQDHTTFDVVLGELRAFLEGRGGELAEPVPFREFVAQARLGVPREEHERYFAELLGDVTETTAPYGLTDVHGDGSRAVQVSLPVAEALAVRVREVARTLGVSPATVFHLAWARVLSVIAGRDDVVFGTILFGRMNSGAAAERVPGLFINTLPVRVRLNGTSVGEALTALRDQMAELMAHEHAPLALAQRAGGVPAGSPLFTSLFNYRHNVAGGGDGGALEGVTPVLHRDTTNYPVVVSVDDDGTSFDLVVEAVAPAEAGRVGRLMHECLAELVGALAGAPETPLSRVRVIDEAEIERVVHSWNDTARPVVESSVPALFAEQVAAAP

SEQ ID NO: 6 - DNA, 1383 bp, Actinoplanes sp.
gccgacatctatccgctggccccgctccaggagggcatcttcttccaccacctcatggcggacgccggccgggccgacgcctacgcgatgccgtacgtgctgcacctggacacggcggagcggctggacgtcctcctcggcgccctccagcgggtgatcgaccgtaacgacatctaccgcaccggcgtggtctcggccggcctgcgcgaaccggtgcaggtggtgtggcggtcggccgtcctgcccgtcgaggaggtggcgctggacggcggccacgacccggtcgagcagttgctcgccgccgccggcgaggagttcgacctgacccgggcgccgctgatccgggcgcacgtggcggcgcatccggacggcggccggctgctcctgctgcgcatccaccacctcgtgcaggaccacacgacgttcgacgtggtgctgggcgagctgcgggccttcctcgagggccgcggcggcgagcttgccgagccggtgccgttccgcgagttcgtggcgcaggcgcggctcggtgtgccgcgcgaggagcacgagcggtatttcgcggagttgctcggcgacgtcaccgagaccaccgcgccgtacggcctgaccgacgtgcacggcgacggctcccgggccgtccaggtctcgctgccggtcgccgaggccctcgccgtccgcgtccgcgaggtggcccggacactcggcgtcagcccggccaccgtcttccacctggcctgggcgcgcgtgctgagcgtcatcgcgggccgcgacgacgtggtgttcggaaccatcctcttcggacggatgaactcgggcgccgccgccgaacgcgtgcccggcctgttcatcaacacgctgccggtgcgggtgcgactgaacggcacgagcgtgggggaggcgctgaccgccctgcgcgaccagatggccgagctgatggcgcacgagcacgcgccgctcgcgctggcgcagcgggccggcggcgtgcccgcgggcagtccgctgttcacgtcgctgttcaactatcggcacaacgtcgcgggcggcggcgacggcggagcgctcgagggcgtcacgccggtgctgcaccgcgacaccacgaactatcccgtggtggtctcggtcgacgacgacggcacgagcttcgacctggtggtggaggcggtcgcgcccgcggaggcgggtcgcgtcgggcggctcatgcacgaatgcctggccgagctggtgggcgccctggccggtgcgccggagacgcccctgtcccgcgtgcgggtgatcgacgaggccgagatcgaacgggtcgttcacagctggaacgacacggctcgcccggtagtggagtcgtcggtgccggcgttgttcgccgagcaggtggcggctgcgccg

SEQ ID NO: 7 - protein, 445 aa, Actinoplanes sp.
ADIYPLAPLQEGIFFHHLMTEGDATDVYLLPRILGFGGRPELDAFLGALQQVVDRHDVYRTAIAWQNLREPVQVVHRHATLPVTEVTPDQLHAAATGGRLPLDHAPLLSVHIAPEPDGGWLALLRMHHLVQDHTALDIVLDEIRTILAGATDHLPPPVPFRDFVAQARLGVSRAEQERYFAGLLGDVTETTAPYGLADVTNDGTASVRAEVELDAALAARLRDLARDRGVSPATVFHLAWARVLAAVADREDVVFGTVLFGRMASGARRVPGLFMNTLPVRVRLSGTAAEALGQVRDRLAELMAHEHAPLALAQQASGLPAGSPLFTSLFNYRYARPPAATPDDPLAGVRTLFAWERNNYPVTVSIDDDGTGFAVTVDVVAPADADEVVRLLRTTLTRLAAALERTPEMPVADVRPGRVSRPAAGRAVLVPVPAGERATGAGRAP

SEQ ID NO: 8 - DNA, 1335 bp, Actinoplanes sp.
gccgacatctaccccctagccccgctgcaagagggcatcttcttccaccacctcatgaccgagggcgatgccaccgacgtctacctcctgccgcggattctcggcttcggcggccgtcccgagctggacgccttcctcggggccctgcagcaggtggtggaccgccacgacgtctatcgcacggccatcgcctggcagaacctgcgcgagcccgtccaggtcgtgcaccgccacgccaccctgcccgtcaccgaagtcacccccgaccagctgcacgccgccgccaccggcggccggctcccgctcgaccacgcacccctgctcagcgtccacatcgcacccgaacccgacggcggctggctcgccctgctccgcatgcaccacctcgtgcaggaccacaccgccctcgacatcgtcctcgacgagatccgcaccatcctcgccggcgcaaccgaccacctccccccgcccgtaccgttccgcgacttcgtggcgcaggcccgcctcggcgtctcccgcgcggagcaggagcgctacttcgccggcctgctcggcgacgtcaccgagaccaccgccccgtacggcctggccgacgtgacgaacgacggcaccgcatcggtgcgggccgaggtcgagctcgacgcggccctggcggcccggctgcgcgacctcgcccgcgaccggggcgtcagcccggcgacggtcttccatctggcctgggcgcgcgtgctggcggcggtggccgaccgggaggacgtcgtcttcggcaccgtcctgttcggacggatggcctccggcgcccggcgggtgcccggcctcttcatgaacaccctgccggttcgcgtacgcctgtccggaaccgccgccgaggctctgggacaggtgcgcgaccggctcgccgagctgatggcgcacgagcacgcgccgctcgcgctggcccagcaggcgagcggcctgcccgccgggagcccgctgttcacgtcgttgttcaactaccgctatgcccggccgccggccgccacgccggacgatccgctggcgggggtgcgcacgctgttcgcgtgggaacgcaacaactacccggtcaccgtgtcgatcgacgacgacggcaccggattcgcggtcacggtcgacgtcgtggcgccggccgacgccgacgaggtcgtccgcctgctccgcacgaccctgacccgcctggccgccgccctcgaacgcactcccgagatgccggtggccgacgtgcggcccggccgcgtgtcccggcccgccgccggccgcgcggtgctcgtgccggtcccggccggcgagcgggcgaccggcgcgggccgggctccg

SEQ ID NO: 9 - protein, 461 aa, Actinoplanes sp.
ADVYPLAPLQEGIFFHHMMADRDSADVYVTPTVVEFDSRDRLDGFLAALQQVVDRTDVYRTSVVWQGLREPVQVVWRHARLPVDEVVLRDDLDPVEQLNALGTAWMDLSEAPLVQAVVAARPGDPQRWLAVLRIHHLVQDHTALDILLEELAAYLAGRGGDLPEPVPFREFVAHTRLGVPREEHERYFAGLLGDVTETTAPYGLLDVHSGGLASAQAHLRLDGPLGRRVAAFAREHGVSPATLFHLAWARVLGTLAGRDDVVFGTVLFGRMNSGAGADRVPGLFINTLPVRVRLGAPVGDALDGLRDQLIELIAHEHAPLAVAQQAANLFGRPLFTSIFNYRYARGAEPAGAALDGIRLLSARDLTNYPLAVAVDAEGDTFSLTVDAVAPADPVQVGELLVTALRNLTRTAENAPGTPLAAVGVLGEDELSRVVSGWNDTARRVRQASVPELFAERVAAAP

SEQ ID NO: 10 - DNA, 1383 bp, Actinoplanes sp.
gcggatgtgtatccgttggcgccgttgcaggagggcattttcttccatcacatgatggccgaccgggattcggcggacgtgtatgtgacgccgacggtggtggagttcgactcccgggaccggttggacggcttcctggccgccttgcagcaggtcgtcgaccgtacggatgtgtatcggacgagtgtggtgtggcaggggctgcgcgagccggtgcaggtggtgtggcggcacgcgcgcctgcccgtcgacgaggtggtgctgcgggacgacctcgacccggtcgagcagctgaacgcgctcggcacggcctggatggacctgtccgaggcgccgctggtgcaggccgtcgtcgccgcccgccccggcgatccgcagcgctggctcgccgtgctgcgcatccaccacctcgtgcaggaccacaccgccctcgacatcctcctcgaggagctggcggcgtacctggccggccgcggcggcgacctgcccgagccggtgccgttccgcgagttcgtcgcgcacacccgcctcggcgtgccccgcgaggagcacgagcgctacttcgccgggttgctcggcgacgtcaccgagaccaccgcgccgtacgggctcctcgacgtgcacagcggcggtctcgcctcggcgcaggcccacctgcggctggacggcccgctcggccggcgcgtggccgccttcgcccgggaacacggcgtcagcccggcgacgctcttccacctcgcgtgggcgcgggtgctcggcacgctggccgggcgtgacgacgtcgtcttcggcacggtcctgttcgggcggatgaactcgggcgccggcgccgaccgcgttcccggcctgttcatcaacacgctgccggtgcgggtgcggctcggcgcgcccgtcggcgacgccctcgacggcctgcgtgaccagctcatcgagctcatcgcccacgagcacgcgccgctggccgtggcccagcaggccgcgaacctcttcggccggccgctcttcacctccatcttcaactatcggtacgcccggggggccgagccggccggcgccgcgctcgacggcatccgcctgctctccgcccgcgacctcaccaactatccgctggcggtggccgtcgacgcggagggcgacacgttctcgctcaccgtcgacgcggtggcgccggccgaccccgtgcaggtcggcgagctgctcgtcaccgcgctgcgcaacctgacccggaccgccgagaacgcgcccggaacgccgctggccgcggtcggcgtgctgggcgaggacgagctgagccgggtcgtctccggctggaacgacaccgcccgccgggtccggcaggcgtcggtgcccgagctcttcgcggagcgggtggcggccgcgccc

SEQ ID NO: 11 - protein, 462 aa, Actinoplanes sp.
ADVYPLAPLQEGIFFHHMMADRDSADVYVTPTVVEFDSRDRLDGFLAALQQVVDRTDVYRTSVVWQGLREPVQVVWRHARLPIDEVELHEGTDPAEQLIALATERVDLDRAPLIRTTTAAVPGSGRWLALLRIHHLVQDHTTLDVLLGELRAFLEGRGDELPEPVPFREFVAQARLGVPREEHERYFAELLGDVTETTAPYGLTEVHGDGSAAVHSRREVDDDLAARLHRLARSLGVSPAALFHLAWARVLGAVSGRDDVVFGTVLFGRMNSGAAADRVQGLFINTLPVRVRLAAGSTRDALTGLRDQLAGLLVHEHAPLALAQRAAGITDGSPLFASIFNYRHNQDDPAASAGLEGIRTVYSAEHTNYPLDASIDVTGDRFAITVNAVAPADAARIAELMHTCLGHLADVLEDAPETPLSWVSPLSAEDLGRIVGDWNETRRAVTRASVPELFAKQVAATP

SEQ ID NO: 12 - DNA, 1386 bp, Actinoplanes sp.
gcggatgtgtatccgttggcgccgttgcaggagggcattttcttccatcacatgatggccgaccgggattcggcggacgtgtatgtgacgccgacggtggtcgagttcgactcccgggaccggttggacggcttcctggccgccttgcagcaggtcgtcgaccgcaccgatgtctaccgcaccagcgtggtgtggcaggggctgcgcgagccggtgcaggtggtgtggcggcacgcgcgcctgccgatcgacgaggtcgagctgcacgagggcaccgatccggccgagcagctgatcgcgctcgccaccgagcgggtggacctcgaccgcgcgccgctgatccgcacgacgaccgcggccgtgcccggatccggccggtggctcgcgcttctgcgcatccaccacctcgtgcaggaccacaccaccctggacgtgctgctcggcgagctgcgggccttcctcgagggccgcggcgacgagcttcccgagccggtgccgttccgcgagttcgtggcgcaggcgcggctcggtgtgccgcgcgaggagcacgagcggtacttcgcggagttgctcggcgacgtcaccgagaccaccgcgccgtacggcctgaccgaggtgcacggcgacggttcggccgccgtgcacagccggcgcgaggtggacgacgacctcgccgcgcgcctccaccggctggcccggtcgctcggcgtcagcccggcggcgctcttccacctcgcgtgggcgcgggtgctcggcgccgtgtcgggccgggacgacgtcgtcttcggcacggtcctgttcgggcggatgaactccggcgccgccgccgaccgcgtgcagggcttgttcatcaacacgctcccggtgcgcgtgcggctcgccgccggcagcacccgcgacgccctgaccgggctgcgggaccagctggccgggctgctggtgcacgagcacgcgccgctcgcgctggcgcagcgcgcggccggcatcaccgacggcagcccgctgttcgcgtcgatcttcaactaccgccacaaccaggacgacccggcggcgtcggccgggctcgagggcatccgcacggtctacagcgccgagcacaccaactacccgctcgacgcctcgatcgacgtcaccggcgaccgcttcgccatcaccgtgaacgcggtggcgcccgcggacgccgcgcggatcgctgagctgatgcacacctgcctcggccacctcgcggacgtgctcgaagacgcgccggagacgccgctgtcgtgggtcagcccgctgagcgcggaggatctcggccgcatcgtgggcgactggaacgagacgcggcgcgcggtcacccgcgcgtccgtgccggagctgttcgccaagcaggtggccgccacgccg

SEQ ID NO: 13 - protein, 461 aa, Actinoplanes sp.
ADVYPLAPLQEGLLFHSLMDGGDDVYVLPAVLGFDSRSRLDAFLAALQHVIDRHDTYRTAVVHDGLREPVQVVWRRATLPVEEVTLTAGADPVQELLATAPVEFALDRAPLLRVRCAARPDGGGWLALLQIHHLVQDHATLDAMLAEIQAFLAGRGGELAAPEPFRGYVARARLAGAPAEHRAYFSRLLGDVTESTAPYGLTDARDARPTGKAHREVDRRLAARVRATASELGVSPATVFHLAWARVLGTLAGRDDVVFGTVLLGRLGAGARSGRALGPFINTLPVRVRLAAAGSRETLAGLRAQLAELIGHEHAPLTLAQAASGVPGGTPLFTSILNYRQGPPAGDDTGDEEIEGIELLSTEERSNYPVAVSVDDDGSGFRLTVDAAQPAAPDRVAELLHTCLHRLTDALAGTPDVEPARIDVLGEAERREVLRTPNATARDVAAATLPAIVGEWARTTP

SEQ ID NO: 14 - DNA, 1383 bp, Actinoplanes sp.
gccgacgtctatccgctcgcgccgctgcaggaggggctgctcttccacagcctgatggacggcggcgacgacgtgtacgtgctgccggccgtcctcggattcgattcgcggtcccgcctcgacgcgttcctggccgcgctgcaacacgtgatcgaccggcacgacacgtaccggaccgcggtggtgcacgacggcctgcgcgagccggtgcaggtggtctggcgccgggccacgctgccggtcgaggaggtgaccctgaccgcgggcgccgacccggtgcaggaactgctcgccaccgcgccggtcgagttcgcgctcgaccgggccccgctgctgcgggtgcgctgcgcggcccggccggacggcggcggatggctggcgctgctccagatccaccacctcgtccaggaccacgccacgctcgacgcgatgctcgccgagatccaggccttcctcgccggccgcggcggcgagctcgccgcgcccgagccgttccgcggctacgtcgcccgggcccggctcgccggcgcgccggccgagcaccgggcgtacttctcccggctgctcggtgacgtcaccgagagcaccgccccgtacgggctgaccgacgcgcgggacgcgcggccgaccggaaaggcccatcgcgaggtcgaccggcggctggccgcccgcgtgcgggccacggcgagcgagctgggcgtgagcccggcgaccgtgttccatctcgcctgggcgcgggtgctgggcacgcttgccggccgcgacgacgtcgtcttcggcaccgtcctgctgggacggctcggcgccggcgcccggtccgggcgagccctcggcccgttcatcaacaccctgccggtgcgggtgcgcctcgccgccgccggcagccgcgagacgctggccgggctgcgcgcccagctggccgagctgatcggtcacgagcacgccccgctgacgctggcacaggccgcgagcggcgtgcccggcgggacgccgctgttcacctcgatcctcaactaccggcaggggccgcccgccggcgacgacaccggcgacgaggagatcgagggcatcgagctgctctccaccgaggaacgcagcaactacccggtggccgtctccgtcgacgacgacggttcgggcttccggctcaccgtcgacgcggcccagccggccgcaccggaccgcgtcgccgagctgctgcacacctgcctgcaccggctcaccgacgcgctcgcgggcacgcccgacgtggagccggcgcggatcgacgtgctcggcgaggcggagcgccgggaggttctccggacgccgaacgccacggcccgcgacgtggcggcggcgacgctgcccgcgatcgtcggcgagtgggcgcggaccacgccc

SEQ ID NO: 15 - protein, 469 aa, Actinoplanes sp.
ADIYPLAPLQQGMLFHHLMAGDDGEDVYIMPAVVEFDSADRFGAFVDALQHVIDRNDVYRTGVVWDGLREPVQVVWRRAPLPVTEVTLDPAGGDPAAQLHAAAGARMDLNRAPLLDLHVAARPEDGQRLALLRVHHMVQDHMGLEVLLGEVQAFLAGRGDELPDPLPFRDFVAQTRGGVPEAEHRRFFAGLLGDVTEPTAPYGLLDVHRDGVGLVRQERPLDGEVVARLRAVARRLGVSPATVMHVAWARVLGVISGRDDVVFGTLLLGRFSTGADRVPGPFINTLPVRARLGGTGAAAAVAEMRRLLAELLEHEHAPLTTAQQASGLSGNLPLFTALFNYRHNTSPGADPSPAAGPTEGIRPVSMRERTNYPISVAVDDDGEGLGVAVNAIPPVRPEAVCELVATATESLTSALELFLDGGPDTAVGELDVLPPGERSRLLVEWNDTARPVVESSVPALFAERVAAAP

SEQ ID NO: 16 - DNA, 1407 bp, Actinoplanes sp.
gccgacatctatccgctcgcgccgctccagcagggcatgctcttccaccacctcatggccggcgacgacggcgaggacgtctacatcatgcccgcggtcgtggagttcgactcggcggaccgcttcggcgccttcgtcgacgccctccagcacgtgatcgaccgcaacgacgtctaccgcaccggcgtggtctgggacggcctgcgcgagccggtgcaggtggtctggcgccgggcgcccctgcccgtgaccgaggtgacgctcgatccggccggcggcgatcccgccgcccagctgcacgccgccgccggcgcccggatggacctgaaccgggcgcccctgctcgacctccacgtggccgcccggcccgaggacggccaacggctggccctgctgcgggttcaccacatggtgcaggaccacatggggctcgaggtgctcctcggcgaggtgcaggcgttcctggccggccgcggcgacgagcttcccgatccgctgccgttccgcgacttcgtggcgcagacccgcggcggggtgccggaggccgagcaccggcggttcttcgccgggctgctgggcgacgtcaccgagcccaccgcgccgtacggcctgctcgacgtgcaccgcgacggcgtcggcctggtgcgccaggaacgcccgctcgacggtgaggtggtggcccggctccgcgccgtggcccgccggctcggggtgagcccggcgaccgtcatgcacgtcgcctgggcgcgcgtgctcggcgtgatctccggccgcgacgacgtggtcttcggcacgctgctgctgggccggttcagcaccggcgccgaccgggtgcccggcccgttcatcaacacgcttccggtgcgggcccggctcggcggcacgggcgccgcggcggcggtggcggagatgcgccggctgctggccgagctgctcgagcacgagcacgcgccgctgaccacggcgcagcaggccagcggactctccggaaacctcccgctgttcacggcgctgttcaactatcggcacaacacgtcgccgggtgcggacccgtcgcccgcggccggcccgaccgagggcatccgcccggtctccatgcgggagcgcaccaactatccgatctcggtggcggtggacgacgacggcgagggcctcggcgtggcggtcaacgcgatcccgccggtgcggccggaggcggtgtgcgagctcgtggcgaccgcgaccgagagcctgacctcggcgctggagctgttcctcgacggcggtccggacaccgcggtcggcgagctcgacgtgctgccgccgggggagcggtcgcggctgctggtggagtggaacgacacggctcgtccggtggtggagtcgtcggtgccggcgttgttcgccgagcgggtggcggccgcgccg

SEQ ID NO: 17 - protein, 466 aa, Pseudomonas syringae pv. syringae strain B301D
QDIYPLAPLQAGILYHHISAEQGDPYTLKALFALSDRAQLDDFSGALQGVINRHDILRTAVLWEDLDEPVQVVLRQAELHVTELLLDPVDGPVDEQLHQRFDRRHYRLDVRTAPLMRIVFSHDPVNDRWLAMLLCHHLVSDATSLSVILQEIQAHLLGQGNALGEAVPYRNYVAQARLGVSEAQHEAFFRDMLGDIDEPTLPFGLQDVQDRGRNLEEASVTLAAELNLRLRAQARQAGVSVASLMHLAWARVLGNVSACEQVVFGTVLLGRMQAGDGADRALGMFINTLPLRVDIGATTVAEGLKATHERLTALLGHEHAPLVLAQRCSGVAAPTPLFSALLNYRHSVVSDVRQKLPGLGGSSLRHRRPYQLPVDLNVDDLGDAFTLTIQAVQQISATRIGEYMQVALRNLVDALEHTPQAALNSLSILPDDERELLLTGFNDTAHPYPRDVLIHQLIEQQAAQRP

SEQ ID NO: 18 - DNA, 1398 bp, Pseudomonas syringae pv. syringae strain B301D
caggacatctacccgctggcaccgttgcaggcggggattctttaccaccatatcagtgccgaacagggcgacccctacaccctgaaagcactgttcgcgctatctgaccgggcgcaactggacgatttcagtggtgctttgcaaggcgtgatcaaccgccacgatatcctgcgtaccgcggtgctgtgggaagatctcgatgagccggtgcaagtcgtgctgcgtcaagctgaactgcacgtcaccgagttgttactcgaccccgtagacggcccggttgacgagcaattacaccaacgcttcgaccgtcggcattaccgcctcgatgtgcgtactgcgccattgatgcgcattgtctttagccatgacccggtcaacgaccgctggctggcgatgctgctgtgccaccacctggtcagcgacgccacgtccctgtccgtgatactgcaggaaattcaggcgcacctgctcggtcagggcaatgcgttgggtgaggcagtgccatatcgcaactatgtcgctcaggcacgcctgggtgtcagcgaggcacagcacgaagcgttcttccgcgacatgctcggcgatatcgacgagccgacgctgccgttcggtttgcaggatgtgcaggaccgtggccgcaacctcgaagaggccagcgtgaccctggcggccgaactgaacctgcgtttgcgggctcaggctcgacaggcaggcgtcagcgtcgcgagcctgatgcatttggcctgggcacgggtgctgggcaatgtctcggcctgcgaacaggtggtgttcggcacggtactgctgggccgtatgcaggccggtgatggcgccgaccgtgcgttgggcatgtttatcaacaccttgccgttgcgcgtcgatatcggcgcaacgacagtggccgagggattgaaggcgacccacgagcggctgacggctttgctggggcatgagcatgctccgctggtactcgcccagcgttgcagcggcgtggccgcaccgacgccgctattcagtgcattgctaaactaccgacacagtgtcgtgagcgatgttcggcagaagctgccagggctgggcggatcgagtcttcggcaccgaagaccataccaactacccgttgacctgaatgtcgatgatctgggcgatgcttttacgctcaccattcaggccgtccagcaaatcagtgccacgcgcattggcgagtacatgcaggttgcactgcgcaacctggtggacgcactggaacacacgccgcaagcagcgctgaacagcctgtcgatactgccggacgatgagcgcgagctgttgctgacgggcttcaacgacacagcccacccttatccgcgtgatgtactgatccaccaactgatcgaacaacaggccgcccagcgtccg

SEQ ID NO: 19 - protein, 461 aa, Pseudomonas syringae pv. syringae strain 301D
QHIYPLAPLQEGILFHYLLNLERDAYLVRSTIEFDSRARLDAFLEGLQTVIDRHDVLRSSVHWVGLPQAVQVVHRQAQSPIHTLTLRPDEDALSQLDRLSDPGRLRLDLRQAPLLLAYIARDPDSERWLLALIDHHMISDHVTLELILEEIRLLMRGQSAELLPPQPYREFVAQTLASPSSAHEAYFTGRLADVDSPTAPFELLEVQGDGNDVEESELALSSDLCARIRTQARERGMSPAVLFHVAWAQVLARCTGRDDVVFGTAVTGRLQGTLGAERAMGMFMNTLPVRVQLATQSVQELVMATHRDLSELLSHEQASLALAQRCSSVATGVPLFSSLLNYRHQNEDSQLQWPGLRLLDSAERTNYPLCLSVNDYGSDLGLLIHSVQPADPQRLCAMMQCALEQLTDALAHTPQKEVTQLDVLPAAERNLLLETFNQTRQDYPTDLCIQHLFEAQVRTQP

SEQ ID NO: 20 - DNA, 1383 bp, Pseudomonas syringae pv. syringae strain B301D
cagcacatctacccgctggcaccgctgcaagaaggcattctgttccattacctgctgaatcttgaacgcgacgcctatctggtgcgctcgacgatcgagttcgacagccgtgcgcgccttgacgcctttcttgagggcctgcaaacggttatcgaccgccatgatgtcctgcgcagttcagtgcattgggtcggcctgccgcaagcggtgcaagtggtgcaccgccaggcgcaatcgccgatccataccctcaccctgaggccggatgaagacgcactcagccagctggatcgtctaagtgatccaggtcgtctgcgccttgatctgcgacaggccccgctgctgctggcctacattgctcgcgacccagactcggaacgttggttactggcgctgattgatcaccacatgatcagcgaccatgtcaccctggaactgatactcgaagaaattcgcctgttgatgcgaggccagagcgcagaactgctgccaccgcagccttatcgggagtttgtcgcacagaccctcgccagcccttccagtgctcacgaagcttacttcaccggccgcctggccgatgtcgattcacccaccgctccgttcgaactgctggaagttcagggcgacggcaatgacgtcgaagagtccgaactggcattgagcagtgacctctgcgcacgtatccggactcaggcccgggagcgcggcatgtccccggcggtgctgttccatgtcgcctgggctcaggtgctggcacgttgtaccggtcgcgatgatgtggtgttcggcaccgcggtcaccggccgtctgcaaggtacgttaggcgccgagcgggccatgggcatgttcatgaacaccctgccggtccgcgtgcaactggccacgcaaagcgtccaggaactggtcatggccacccaccgtgatctcagcgagttactgagccacgagcaggcatcattggccctggcccagcgttgcagtagcgtggccacgggtgtgccgttgttctccagcctgctcaactaccggcaccagaacgaggacagccagttgcaatggccgggcctgcgcctgctcgacagcgccgagcgcaccaactatccgctgtgcttgtcggtcaacgattacggcagtgacctgggcctgttgatacacagtgtgcaacctgcggatccgcaacgcctgtgcgccatgatgcagtgtgcgttggagcaattgaccgacgcgctggcgcacacaccacagaaggaagttacgcagttggatgtgctgcccgccgccgagcgcaacctgctgctggagaccttcaaccagacccgccaggactacccgacggacctgtgcattcagcacctgttcgaagcacaggtccgcacgcaaccg

SEQ ID NO: 21 - protein, 461 aa, Pseudomonas syringae pv. syringae strain B301D
QDIYPLAPLQDGILFHYLLNRERDAYLMRSMIEFDSRARLDAFLEGLQTVIDRHDILRSSVHWIGLPQAVQVVHRQAQLPVHTLTLTPEEDALSQLDRLSDPGRLRLDLRQAPLLLAYIARDPNSERWLLALIDHHMISDHVTVELILEEIRLLMRGQSADLLPPQPYRDFVAQTLASPSSAHEAYFTRRLADVDSPTAPFELLEVQGDGNDVEEAKLALNSDLCIRIRTQARERGMSPAVLFHVAWAQVMARCTGRDDVVFGTVVTGRLQGTAGAERAMGMFMNTLPVRVQLTTQGAQELVMATHRDLSELLSHEQASLALAQRCSSVATGVPLFSSLLNYRHQGEDNRLQWPGMRLLDGTERTNYPLCLSVNDYGSELDLIIHSMQPANPQRLCAMMQCALEQLTDALAHTPQMAVTQLDVLPAAERNLLLETFNQTRQDYPTDLCIQHLFEAQVRTQP

SEQ ID NO: 22 - DNA, 1383 bp, Pseudomonas syringae pv. syringae strain B301D
caggacatctacccgctggcaccgcttcaggacggcatcctgttccattacctgctgaatcgtgagcgcgacgcctacctgatgcgctcgatgatcgagttcgacagccgtgcgcgccttgacgcctttcttgagggcctgcaaacggttatcgaccgccatgacatcctgcgcagctcggtgcattggatcggcctgccgcaagcggtgcaagtggtgcaccgccaggcgcaattgcctgtccataccctcaccctgacgccggaggaagacgcactcagtcagctggaccggctaagtgatccaggtcgtctgcgcctggatctgcgacaggccccgctgctgctggcctacattgctcgcgacccaaactcggaacgttggctactggcgctgattgatcatcacatgatcagcgaccacgtcaccgtagaactcatactcgaagaaattcgcctgttgatgcgaggccagagcgcagacctgctgccaccgcagccttaccgggattttgtcgcacagaccctcgccagcccttcaagtgcccacgaagcttacttcacacgccgcctggctgatgttgattcacccaccgctcctttcgaactgctggaagttcagggcgacggcaatgacgtcgaagaggccaaactggcattgaacagtgacctttgcatacgtattcggactcaggcccgggagcgcggcatgtccccggcggttctgttccatgtcgcctgggcccaggtgatggcccgttgtaccggtcgcgatgacgtggtgttcggcaccgtggtcaccggccgcctgcaaggtacggccggcgccgagcgggccatgggcatgttcatgaacaccctgccggtccgcgtgcaactgaccacacaaggcgcccaggaactggtcatggccacacaccgtgatctcagcgaattactgagccacgagcaggcgtcactggccctggcccaacgttgcagtagcgtggccacaggggtgccgttgttctccagcctgctcaattaccggcaccagggcgaggacaaccggttgcaatggccgggcatgcgcctgctcgacggcaccgagcggaccaactatccgctgtgcttgtcggtcaacgattacggcagtgagctcgacctgataatacacagcatgcagcccgcaaatccgcaacgcctgtgcgccatgatgcagtgcgcgctggagcaactgaccgacgcgctggcgcacacaccgcagatggccgttacgcagctggatgtgctgcctgccgccgagcgcaacctgctgctggagaccttcaaccagacgcgccaggactacccgacggacctgtgcattcagcacctgttcgaagcacaggtccgcacacaacct

SEQ ID NO: 23 - protein, 470 aa, Pseudomonas syringae pv. syringae strain B301D
QDIYPLSSLQEGILFHHLLQSEGDAYLMRTLATFDSRALLDKFLGALQVVINRHDIMRSSLRWQGLPQPVQVVHRQAQLPVIQLDTAPGEDALQMLRERTNTYHMRLDLQQAPLIAAYITYDTRQEKWLMALLDHHLISDNVTLRLIMGEIQAVMDGRADALPPSQPYRNFIARAACVSQAEHEAYFRQLLGDVDTTTAPYGVLDVRGGDATILRSVQDLSDDLSARIHSTARAQGVPTSVLFHAAWGLVVAATSGRDDGIFGTVLSGRSQGTSGANHALGMFINTLPMRIRLQQNSVRDIVQDAYQQLSGLLTHERAPLALAQRCSAVDASLPMFTVILNCRHGDLVNTAGENIEDMGEEQEGVHFLGSETRTNYPIEIAVANEANGFSLTAQSIDGIDPHRIAAYLGQAVAELVNALEQDPARLASSLEVIPQAERQLLLNDFNDTASDFAPAVPIHALFEDQVRRNP

SEQ ID NO: 24 - DNA, 1410 bp, Pseudomonas syringae pv. syringae strain B301D
caggatatctatccactgtcatcgttgcaggaaggcatcctgtttcaccatctgttgcagtcagaaggcgacgcctacctgatgcgcaccctagcgacgttcgacagccgtgcgttgctcgacaaattcctcggcgcgctgcaagtggtgatcaaccgccacgacatcatgcgcagctccctgcgctggcagggtctgccgcagccggtgcaggtggtgcatcgtcaggcacagttgccagtcattcagctcgacaccgcgccgggtgaagacgccttgcagatgctgcgcgaacgcaccaatacctaccatatgcgcctggacctgcaacaggcaccgctgatagccgcgtatatcacttacgatacccggcaggaaaagtggctgatggcactgctggaccatcacctgatcagtgacaacgttaccctgcgcctgatcatgggcgagatccaggcagtgatggacggacgcgccgacgccctgccgccatcccagccgtaccgcaacttcatcgcccgggccgcgtgtgtttcccaggccgaacatgaagcctacttccgtcaactgctgggcgatgtcgataccaccactgcgccctacggcgtgctcgatgtgcgtggcggcgatgcaacaatcctgcgttcggtgcaggatctgagcgacgatctcagcgcgcggatccacagcaccgcacgggcccagggcgtaccaacctcggtgctgttccacgcggcatgggggttggtggtggccgccaccagcggccgtgacgacgggattttcggtaccgtgctttcgggacgttcccagggcacctccggcgccaaccatgcgctggggatgttcatcaataccttgccgatgcgtattcgcctgcaacaaaacagcgtgcgcgatatcgttcaggatgcctaccaacagctgagcgggctgctgacccacgagcgggcgcctctggccctggcgcagcgctgcagcgcagtggatgcatcgctgccgatgttcaccgtgatcctcaattgccgtcacggtgatctggtgaacactgccggggaaaacatcgaggacatgggcgaggagcaggaaggtgttcatttcctgggctccgaaacccgtaccaactacccgatcgaaattgccgtagccaatgaggccaatggcttttcgctgaccgcgcagtctatagatggcatcgatccgcaccggattgccgcctacctcggtcaggcggttgccgagctggtcaacgcccttgagcaggatccggcacgcctggcgagcagtcttgaagtgattccgcaagccgagcggcagctgctgctcaacgacttcaacgacaccgccagcgacttcgcaccggccgtaccgatccatgccttgttcgaagaccaggtccgccgcaatcct

SEQ ID NO: 25 - protein, 471 aa, Pseudomonas syringae pv. syringae strain B301D
QDIYGLVPLQEGILYHHLSSGEGDPYLLQALLRFNSFEQLLDFTEALQQVIDRHDILRTAVAWEELDEPVQVVWRQARLRVEAFLPHPDQGDVAAQLQAQFDPRRIRMDLHQAPMMRLHYAEDPVNQSWVAVLLFHHLIDDATSLALLGAEIEAFRQGRGNHLPASVPYRNHVAQARLSMSREEHEAFFRDMLADVDEPTLPFGLQDVQGDGSGVDEALLPVDPELAGRLRAHARRLGVSNASLHHLAWAQVVGRLSGRQDVVFGTVLMGRLLSGQGAERALGMFINTLPLRVAAGEQGVEAAVRTTHARLAALVSHEHAPLSLAQGCSGVAAPTPLFSALMNYRHVAVGAQTPQQAGQTRTWAGVEVLGGEERTNYPLSLSVDDLGDSFGLTVQAVAGIDARRICGYMHTVLEQLADALDSRPDAPLHSLDWLPAHERRQLLEDFNAFDRDYPQDLLLHQLFEAQAAAQP

SEQ ID NO: 26 - DNA, 1413 bp, Pseudomonas syringae pv. syringae strain B301D
caggatatctatgggctggtgccgttgcaggagggcatcctctaccaccatttgtccagtggagagggtgatccgtacctgttgcaggcgttgttgcggttcaatagcttcgagcagctgctggacttcacggaggccttgcagcaagtcattgaccgtcacgacattctgcgcacggcggtagcctgggaagagctcgacgagccagtgcaggtggtctggcgtcaggcacggctgcgggtcgaggcgtttttgccgcacccggaccagggtgatgtcgcggcccagttgcaggcacagttcgatccacgacggatacgcatggacctgcatcaggcaccgatgatgcgcctgcattacgccgaagatcctgtcaaccaaagctgggtggcggtgttgctgttccatcacctgatcgatgacgccacctcgctggccctgctgggcgcagagatcgaggcgttcagacaaggacgcggcaaccatctgccagcgtcggtaccgtatcgcaatcatgtggctcaggcgcgattgagcatgagccgcgaggagcacgaagcgtttttccgcgacatgctcgccgatgttgacgagccgaccctgccgttcggcctgcaagacgtgcaaggcgacggcagcggagtggacgaagccctgttgccggttgacccggagctggccggacgcctgcgcgcccatgcccggcgcctcggcgtcagcaatgccagcctgcatcaccttgcgtgggcacaggtggtcgggcgcctgtcagggcgtcaggatgtggtcttcggtaccgtgctgatgggccgcctgctcagtggtcaaggcgccgagcgggccttgggcatgtttatcaataccttgccattgcgcgtcgctgccggagagcagggcgttgaggccgctgtgcgcaccacccacgcacggttggcggcgttggtcagtcatgagcatgcaccgttgtccctggcccagggatgcagcggcgtggcagcaccgacacctttattcagtgcgctgatgaactaccgccatgtcgcggtcggcgcccagacacctcaacaggcggggcagaccagaacctgggccggtgtcgaggtactcggcggtgaagagcgcaccaactacccattgtcgctatcagtggacgacctcggcgattccttcggcctgaccgttcaggcggtggccggaatcgatgcccggcgcatctgcggttacatgcacaccgtactggagcaactggccgacgcgctggacagtcggcccgacgctcccttgcacagcctcgactggttgcccgcgcacgagcgtcgtcaattgctggaggacttcaacgcgttcgatcgcgactacccgcaagaccttctgctgcaccaactgttcgaggcccaggcggctgcgcaaccg

SEQ ID NO: 27 - protein, 466 aa, Pseudomonas syringae pv. syringae strain B301D
QDIYPLAPLQEGILYHHIAAEQGDPYLLQATFALRDPAHVRRFAEGLQAMIDRHDILRTALVWEGLEQPLQVALRRVELSMEQITLAQDGGDIVAQLQRRFDPREYRLDLNHAPLIHLAFAEDSENQRWVAILLFHHIALDHTALDVMSVEMLAHLQGTAAQLPAAMPYRNYVGQARLGVSRKQHEAFFREMLGDVDEPTLPFGLLEVQGDGQGIKEVRHALPADLCQRLRTQARQLGVSAASLHHLAWARVLGSASGRDDVVFGTVLLGRMQSGLGADRALGMFINTLPLRVDVGNCAVRQAVRQTHARLTALLGHEHAPLVLAQRCSAVAAASPLFSALLNYRHSPAREASGGERWADTQVLGVRERTNYPLTLSVDDQGDGFLLSVQVAAGFDGQRICGYMQTALSHLVEALESAPDSAVRDLSVVPETERRQLLVAFNDTARDYPQQQTVHGLFEAQVRAHP

SEQ ID NO: 28 - DNA, 1398 bp, Pseudomonas syringae pv. syringae strain B301D
caggacatctacccacttgcaccgttgcaggaaggcattctttatcaccatatcgcggctgaacagggcgacccgtatctgctgcaagcgaccttcgccctgcgcgacccggcacacgtacggcgctttgccgaaggcttgcaggcgatgatcgatcgccatgacatcctgcgcacggcgctggtctgggagggtctggaacaaccgctgcaagtggctctgcgccgggtcgaactgtcgatggagcagatcaccctggcccaggacggcggcgatatcgtcgctcagttgcaacgccgttttgatccacgcgagtaccgcctcgatctcaatcatgccccgctgattcacctggcctttgccgaggactcggagaaccagcgctgggtggcaatcctgctgttccaccacattgcccttgaccacaccgcgctggacgtgatgagcgtagaaatgctcgcccacttgcaaggcaccgcagcgcaactgccggcggcgatgccttaccggaattacgtgggtcaggctcgtctgggcgtaagtcgcaagcaacacgaagcgtttttccgcgagatgctcggtgatgtcgatgaaccgaccctgccattcggcctgcttgaagtgcagggtgacgggcagggtatcaaggaggttcgccacgctcttccggctgatctgtgtcagcgtctgcgtactcaggcgcggcaactgggggtgagtgcagccagcctgcatcacctggcctgggcgcgagtgctgggcagcgccagcggacgtgatgacgtggtcttcggcactgtgttgctgggacgtatgcaaagcggcctgggtgctgaccgcgccctggggatgttcatcaacaccttgccgctgcgggtcgatgtcggtaactgcgccgtgcgtcaggccgtgcgtcagacccacgcccgattgaccgcattgctcggccacgaacatgcgccgttggtgttggctcaacgttgcagcgcggtggccgcagcctcgccgctgttcagcgcgctactcaactaccgccacagcccggcacgcgaagccagcggtggcgagcgttgggcggatacccaggtgctcggcgtacgcgaaaggaccaactacccgttgaccctgtcggtggacgatcagggcgacggtttcctgttgagcgtacaggtggctgccggattcgatggccagcggatctgcggttatatgcagaccgccttgagccatctggtcgaggcccttgaatccgcgccggactcagccgtgcgtgacctgtcggtggtgccggaaacagaacgccggcaactgctggtggcgttcaacgacaccgcgcgggattacccgcagcaacagaccgtgcatggcctgttcgaagctcaggtgcgtgcgcaccca

SEQ ID NO: 29 - protein, 468 aa, Pseudomonas syringae pv. syringae strain B301D
QDIYPLAPLQAGILYHHISAEEGDPYTLKALFGLCDRARLDDFSGALQGVINRHDILRTAVLWEGLDEPVQVVLRRAELQVTELFLDPADGPVDEQLHQRFDPRHYRLDVRQAPLMQIVFSHDPLNDRWLAMLLFHHMVNDATSLQVVLHEVQAHLLGQSAVLGEPVPYRNYVAQARLGVSDARHEAFFRDMLGDIDEPTLPFGLQDVQDGGRDIEEACVTLTTELNQRLRAQARQAGVSAASLMHLAWARVLGSVSAREQVVFGTVLLGRMQAGDGADRALGMFINTLPLRVDVGAATVVEGLKATHEQLTALLGHEHAPLVLAQRCSGVAAPAPLFSALLNYRHMAQTRMTAQYEAAWQGIETFNAKDNTNYPLTLNVDDTGDGFRLSVLITADIGAQRLCDYMQAAIEQLVQALEQTPAAPLNSLTILPAAERQKVLVDFNATATDSPRQMTVHALFEAQAERTP

SEQ ID NO: 30 - DNA, 1404 bp, Pseudomonas syringae pv. syringae strain B301D
caggatatttatccactggcgcctttgcaggccgggattctctatcaccacatcagtgccgaagagggtgacccctacaccttgaaagcgttgttcgggttatgtgaccgggcgcgactggacgatttcagcggcgctctgcaaggcgtgatcaatcgccacgatatcctgcgcaccgccgtgctgtgggaaggtctcgacgaaccggtgcaggtggtgctgcgcagggccgaactgcaagtcaccgagctgttcctcgacccggcggacggcccggtcgatgagcaattgcaccagcgcttcgatcctcggcattaccgcctcgatgtgcgtcaggcgccactgatgcaaattgtcttcagccacgacccgctcaacgaccgctggctggcaatgctgctgtttcaccacatggtcaacgacgccacctcgctgcaggtggtgctgcatgaagttcaagcgcacctgcttggccagagcgcagtcttgggcgagccagtgccgtatcgcaattatgttgcccaggcccgcttgggtgtcagcgatgcccgacacgaagcgtttttccgcgacatgctcggcgatatcgacgaaccgacgctgccattcgggttacaggatgttcaggacggcggccgcgacatcgaagaggcctgtgtgaccctgaccacagaactgaaccagcgtctgcgggctcaggccaggcaggcgggcgtcagcgccgcgagcctgatgcacctggcctgggcgcgggtgctgggcagtgtctcagcccgcgagcaggtggtgttcggtacggtattgttgggccgtatgcaggccggtgatggcgccgaccgtgccctgggcatgtttatcaacaccttgccgctgcgcgtcgatgtcggcgcagcaacggtggtcgagggattaaaagcaactcacgagcaactgaccgccttgctgggccatgaacatgcgccgctggtgcttgcccaacgctgcagcggtgtcgctgcaccggcacccctgttcagcgcgctgctcaactatcggcacatggcgcagaccaggatgacggcgcagtacgaagccgcctggcaagggatcgagaccttcaacgccaaggacaacaccaattacccgcttaccctgaatgtcgacgatacaggcgatggcttccgattgtcggtgctgatcactgccgatatcggtgcgcagcggctgtgcgattacatgcaggcggcgattgagcaactcgttcaggctctggagcaaacgcctgcggcgccgctgaacagcctgaccatcctgccagcggcggagcgtcagaaggtactggtcgacttcaacgccacggctaccgattcaccacggcagatgaccgtgcacgcgctattcgaggctcaggctgagcgcacgcct

SEQ ID NO: 31 - protein, 468 aa, Pseudomonas syringae pv. syringae strain B301D
KDIYPLAPLQEGILYHHLAAENGDPYVLQTLFEIADRQRLNAFVDALQSVIDRHDILRTAVLWQGLQTPMQVVWRQARLHLEQIELDPADGAVINQLQQRFDPRHSRLDMSQAPLLRVVFAEDSPNQRWVAILLFHHMVLDHTAMEVVLHDMQAHLLGQADQLDAAIPYRNYVAQARLGVSREAHEAFFRELLGDIDEPTLPFGLLDVQGDGRDIEDSSLTLDAQLNLRLRAQARQLGVSAASLVHLAWAQVLGKVSGRQTVIFGTVLMGRMQSGEGADRALGMFINTLPLRVDLAGQGARDGVRATHARLTALLGHEHAPLVLAQRCSGVAAPLPLFSSLLNYRHSAVAEPSDESIEAWRGIEVRSGEERTNYPLTLNVDDLGDGFRLSVLVTGKVGAGRVCGYMQTALENLLVALEQSPDTALDSLPILPAAEREQLLVRFNDTELDYPHEQTIHGLFEAQAERTP

SEQ ID NO: 32 - DNA, 1404 bp, Pseudomonas syringae pv. syringae strain B301D
aaggacatctacccgctggcaccgttgcaggaggggattctctaccaccatctggccgccgaaaacggcgatccgtacgttctgcaaacactcttcgagatcgctgatcgtcagcgtttgaacgcctttgtcgacgccttgcaaagtgtgatcgaccgtcacgacatcttgcgtaccgcagtgctgtggcaaggtctgcaaacaccgatgcaggtcgtctggcggcaggctcgcctgcatctggagcagatcgaactcgacccggccgacggcgcggtcatcaatcagttgcaacagcgcttcgaccctcgccattcgcggctggacatgagccaggcaccgctgctgcgtgtggtcttcgccgaagattcgcccaaccagcgctgggtggcaatcctgctgttccatcacatggtcctcgaccatacagcgatggaggttgtgctgcatgacatgcaggcacacttgctcggccaggctgaccaactcgacgccgccattccgtatcgcaattatgtggcccaggctcgcctgggcgtgagccgcgaggcccatgaagcgttcttccgcgaactgctgggggacatagacgaaccgacgctgccgttcggcctgctggatgtgcagggcgacggccgcgatatcgaagacagcagcctgacactggacgctcagctcaatctgcgcctgcgtgcccaggcccgccagttgggagtcagtgccgcgagcctggtacatctggcgtgggctcaggtgttgggcaaggtatcgggcaggcaaacagtgatcttcggcactgtgctgatgggccgcatgcagagtggcgaaggtgccgaccgggcgttggggatgttcatcaataccttgccgctgcgcgtcgatctggccgggcagggtgcacgggacggggtgcgggcgacccatgcccggctcaccgcgttgctcggccacgaacacgcgccgttggtgctcgcgcagcgttgcagcggcgtggcggcgccattgccactgttcagttcgctgctcaactaccgtcacagtgccgtggcagagccctcagacgagtcgattgaggcctggcgcggtatcgaggtgcgcagtggcgaggaacgcaccaactacccgctgaccttgaacgtcgatgacctgggcgatggtttccggttatcagtgctggtaaccggcaaggtcggtgccgggcgggtttgcgggtatatgcagaccgcgctggaaaacctgctcgttgcgctggaacagtcgccggacactgccctcgacagcctgccgatcctgccggctgccgagcgcgagcagttgctggtgcggttcaacgataccgagctggattacccgcacgaacagaccattcatggcttgttcgaagcacaggccgagcgcacgccg

SEQ ID NO: 33 - protein, 468 aa, Pseudomonas syringae pv. syringae strain B301D
QDIYPLAPLQEGILYHHIAAGQGDPYVLQAQFTIASRERFDEFTTALQQVVDRHDILRTSVIWEGLAEPVQVVWRKADLVLSEITIDPAAGTASEQLQQRFDPRHYRLDISQAPLLRLAFTHDVVNQCWVAMLLFHHIAIDHAALELVQHEIHAHLYGQAHTLGEPVPYRNYVAQARLGVTNKQHKGFFREMLGDVDEPTLPFGLHDVRGDGHGVEEAHQTLPIELSQRLRTQARLQGVSAASLHHLAWAQVLGRLSGRNDVVFGTVLLGRMRGGEGARRALGMFINTLPLRVAVGEQDVRAGVKATHAHLTALLGHEHASLALAQRCSGVSAPTPLFSALLNYRHSSAEAVTGQAVQLWEGIEVRGGEERTNYPLILSLDDLGEGFSLNVQAVAGIGAQRVCAYMQTALESLVHALEQTPQAPLNRLPILPADEREQLLVAFNDTALDYPQQQTIHGMFEAQVERTP

SEQ ID NO: 34 - DNA, 1404 bp, Pseudomonas syringae pv. syringae strain B301D
caggacatctacccgctggcgcccttgcaggaaggcattctttatcaccatatcgccgccgggcagggcgacccttacgtactgcaggcacagttcacgattgccagccgggaacgtttcgacgagttcaccacggccttgcaacaggtcgtcgatcgccacgacatcctgcgtaccagcgtcatatgggaaggcctggccgagccagtgcaagttgtctggcgcaaggctgatctggtgctcagcgaaatcaccattgaccctgccgcaggcaccgccagcgagcaattgcaacaacgcttcgacccacggcattaccgcctcgatatcagccaggcacccttgctgcgactggccttcacccacgacgtggtcaaccagtgctgggtagcgatgctgctgtttcaccacatcgccatcgaccatgcagccctggaactggtccagcacgaaatccatgcgcacctgtacggccaggcccacaccctgggcgagccggtgccttaccgtaactacgtggcccaggcccgactcggcgtcaccaacaaacagcacaaaggttttttccgcgagatgctcggcgacgtcgatgaaccgacgttgccgttcggcctgcacgatgtgcgcggcgacggccatggcgtcgaagaagcacaccagactttgcccatcgagctgagccagcgtttgcggactcaggcccggctgcaaggagtgagcgcggcgagcctgcatcacctcgcctgggcacaggtgctcgggcgccttagcgggcgtaatgatgttgtgttcggcacggttctgctggggcgcatgcgcggtggcgaaggggctcgccgggccttggggatgtttatcaataccttgccgctgcgggtggccgtgggagagcaggacgtgcgcgccggggtcaaggcgacccatgcgcatttgacagcgttgctcggccacgaacacgcctcgctggcgttggcccagcgttgcagcggcgtgtccgcaccgacgccactgttcagcgcgttgctcaactaccgccacagttccgctgaggcggtgactggccaggcggttcaattatgggaagggatcgaggtgaggggcggcgaagaacgcaccaactatccgttgattttgtccctcgatgatcttggcgaaggattcagcctcaatgttcaggcggtcgccggtatcggcgcccagcgagtttgcgcgtatatgcagacggcgctggaaagcctggtgcatgcgctggagcagacgccgcaggcgcctttgaaccgcttgccgatattgccagccgatgagcgtgagcagttgctggtggcgttcaacgacaccgcactggattacccgcagcagcagaccatccatgggatgttcgaggcccaggtcgagcgcacgccg

SEQ ID NO: 35 - protein, 468 aa, Pseudomonas syringae pv. syringae strain B301D
QDIYPLAPLQEGILYHHIAAEQGDPYVLHSQFTLSDRQRLEAFAKALQQVIDRHDILRTAVIWQGLDEPLQVVWRKAELSMEQVHPDPRNGDIARQLRERFNIRHSRLDLTRAPLMQLVFAEDEACQRWVVMLRFHHMALDHTTLEVVRHEIQAYLLDQPDTLGAAIPYRNYVAQARLSVSREEQEAFFRDMLGDVDEPTLPFGVSDVQGDGQPVKEATCVLPSTLSLRLRASARQLGVSAASLHHLAWAQVLGRVSGKQDVVFGTVLMGRMLGGEGTERALGMFINTLPLRVVVNEQGVRAGVRATHARLTELLGHEHASLALAQRCSGVAAPTPLFSALLNYRHSAPASVSEQARQAWQGIDALSSEERTNYPLTLNIDDLGDGFSLNVLAVADIDAQRVCDYMQTVLSHLVEALESAPDSAVCGLPIVPEAERQQLLVAFNDTARDYPQQQTVHGLFEAQVRAYP

SEQ ID NO: 36 - DNA, 1404 bp, Pseudomonas syringae pv. syringae strain B301D
caggacatctacccgctggcgccattgcaggaaggcattctctatcaccatatcgccgcagagcagggcgacccttacgtgctgcactcacagttcacactgtcggatcgtcagcgtctggaagcctttgccaaagcgctgcaacaggtcatcgaccgccatgacatcctgcgcacggcggtgatctggcaggggctggacgagccgctgcaagtggtctggcgcaaggccgagctgagtatggaacaggtccatccggacccgcgcaacggcgatatcgcacgccagttacgagagcgcttcaacattcgtcactcgcgccttgatctgaccagggcgccactgatgcaactggtgtttgccgaagacgaagcctgccagcgctgggtggtaatgctgcgcttccatcacatggcgctggaccacactacgctggaggtggtgcgccatgagattcaggcgtatttgctcgaccagcccgacaccctcggcgcggccataccgtatcgcaattacgtggcccaggcccgcctgagtgtgagccgcgaggagcaagaggcgttctttcgcgacatgctcggcgatgtcgatgaaccgacgctgccgtttggcgtaagcgatgtgcaaggcgacggtcagccggtcaaagaggccacctgtgtgctgccgtcgacgctgagcctgcgtctgcgcgccagtgcccgacaactgggggtgagtgccgccagcctgcatcacctggcctgggctcaggtgctgggcagggtatcgggcaaacaggatgtggtgttcggcaccgtactgatgggccgtatgctgggcggagagggcacggaacgggcactggggatgttcatcaacactctgccactgagggttgtcgtgaacgaacagggcgtgcgcgccggggtcagggcgacccacgcgcgcctgaccgaattgctcggccatgagcatgcatcccttgccctggcccagcgttgcagcggcgtagccgcgccgaccccgttgttcagcgccttgctcaactaccggcacagcgccccggccagtgtgtccgaacaggccaggcaagcgtggcagggtatcgatgccctgagcagcgaagaacgcacaaactacccgctgaccctgaacatcgatgatctgggtgacggcttcagcctcaatgtattggcggtcgcggacatcgatgcccagcgcgtctgcgattatatgcagacggtcttgagccatctggtcgaggctcttgaatccgcaccggattcagccgtatgcggcctgccgatagtgccggaagccgaacgccagcaactgctggtggcgttcaacgacaccgcgcgggattacccgcagcaacagaccgtgcatggcctgttcgaagctcaggtgcgtgcgtacccc

SEQ ID NO: 37 - protein, 468 aa, Pseudomonas syringae pv. syringae strain B301D
QDIYPLAPLQAGILYHHLSAEQGDPYVLQSQFAFASQERLEDFVVALRRVIQRHDILRTALAWEGLDEPVQVVWRHASLIRELLHPDPQGPDVSAQLHQRFDARHYRLDIRQAPMMRLIHAWDEPNQRWLALLLFHHLALDHTALDVLRHEMQACLLGQQAQLGAPVPYRNYVAQACLGLSREEHKAFFRDMLGDVDEPTLPFGLQDVQGDGHAIEEATRVLSAALNLRLRAQARQLGVSVASLAHLAWAQVLGKVSGKQDVVFGTVLMGRMQGGDGADRALGMFINTLPLRVAVGEQGVRAGVQATHARLTALLGHEHAFLALAQRCSGISAPTPLFSALLNYRHSAEARVSEQATLAWQGIETLGGEERTNYPLALNVDDLGDGFSLNVQVSGNVGAMRVCDYMETALEQLLLALEQDPEAPLNSIAILSAAEREQLLVGFNNTALDYPHQQTVHGLFEAQVRNNP

SEQ ID NO: 38 - DNA, 1404 bp, Pseudomonas syringae pv. syringae strain B301D
caggacatctacccgctggcgcccttgcaggcggggattctctaccaccacctcagtgcggaacagggcgacccctatgtactgcaatcgcagtttgcttttgcaagtcaggagcgtctggaggatttcgtcgtggccctgcgacgggttatccaacgccatgacatcctgcgtaccgcgctggcctgggaaggcctcgacgagccggtgcaggtggtctggcgtcacgccagcctgatccgcgagcttctgcatccggacccgcaaggccccgatgtgtctgcacagcttcatcagcgtttcgacgcccggcactatcgtctggacattcgtcaggcaccgatgatgcgtttgattcatgcctgggatgagcccaaccagcgctggctggccttgctgctgtttcatcacctggcgctggaccacacggcgctggatgtgctgcgacacgagatgcaggcctgcctgctgggccagcaagcgcaactgggcgcgccggtgccgtatcgcaattatgtggctcaggcgtgtctgggcctgagccgcgaggagcacaaggcattcttccgcgacatgctgggcgacgtcgacgagccgacgctaccatttggcctgcaggatgtgcagggcgacggtcacgctatcgaagaggcaacgcgggtcttgtctgcggcgctgaacctgcgcctgcgggctcaggcacggcaactgggcgtgagcgttgccagcctggcgcacctggcgtgggcgcaagtcctgggtaaggtgtcgggcaagcaggatgtggtgttcggcaccgtgctgatgggccggatgcagggtggtgatggcgctgaccgggccttggggatgtttatcaacaccttgccgctgcgggttgccgtaggtgagcagggcgtgcgggccggggtgcaggcgacgcatgcgcgactcacggcattgctgggccatgaacacgcgtttctggcactggcccagcgttgcagtggcatatccgcgccgacaccgttgttcagcgccttgctcaactaccggcacagcgccgaggcccgggtgtccgagcaggccacactggcctggcagggtatcgaaaccctcggtggagaggagcgcaccaactacccgctggccttgaatgtcgacgacctgggcgatggtttcagcctgaatgtgcaggtcagcggcaacgtaggcgccatgcgggtgtgtgactatatggaaaccgcgctggagcagctgcttctggcgctggagcaggaccccgaagcgccgctgaacagcattgcgattctttcggctgccgaacgggagcagctactggtcgggttcaataacaccgcgctggattacccgcaccagcagaccgtgcatggcctgttcgaagcacaggtccgtaacaacccg

SEQ ID NO: 39 - protein, 467 aa, Pseudomonas syringae pv. syringae strain B301D
QDIYPLAPLQEGILYHHLSAERGDPYLLQSLFSFDSQEHVDDFARALQFVIQRHDILRTALVWEGLDEPMQVVWREALLVRESFDADPHGSDMAAQLHQRFDARHHRLDTRQAPMMRLIHAWDEPNQRWLALLQFHHLVMDHTTLDVVRYEMQASLLGQEAQLGAAVPYRNYVAQARLGVSQDEHEVFFSDMLGDVDEPTLPFGLQQVHGDGHGIEEDQLAVGADLSRRLRVQARQLGVSAASLVHLAWARLLGQVSGRDDVVFGTVLLGRMQGGDGADRALGMFINTLPLRVTLGSRSVCESVREVHDKLTALLGHEHASLALAQRCSGVVAPAPLFSALLNYRHSSVAVTDEALTAWNGMQSLGDEERTNYPLTLNVDDQGEDFLLTVQTVPLIDAARICAYMQQTLNSLVDALEQAPQTPLHAISILPRKERTQLLEQWNEPGQHYANDTPIHQQFEARAAARP

SEQ ID NO: 40 - DNA, 1401 bp, Pseudomonas syringae pv. syringae strain B301D
caggacatctacccgctggcgccgctgcaggaaggtatcctctatcaccacctcagcgctgaacgcggtgatccgtatttactgcaatcgctgttcagcttcgacagccaggaacatgtcgacgattttgcccgcgccttgcagttcgtgatccagcgtcatgacatcctgcgcaccgcgctggtctgggagggcctggacgaaccgatgcaagtggtctggcgtgaggcgttgctggtgcgcgaaagctttgatgccgacccgcacggtagcgacatggcggcacagttgcaccagcgtttcgatgccaggcaccatcgcctggacactcgtcaggcgccgatgatgcggttgattcacgcctgggacgagccaaaccagcgctggctggccttgctgcagttccatcacctggtcatggaccacaccacgctggacgtggtgcgctacgagatgcaggccagcctgctggggcaggaggcgcaactgggcgcggcggttccgtatcgcaactatgtcgcccaggcacgactgggcgtgagccaggacgagcatgaagtattcttcagcgacatgctgggcgacgtcgacgagccgacgctgccgttcggtttgcagcaggtgcacggtgacggccatggcatcgaagaggaccaactggcggtaggtgcagacctcagccgccgcttgcgggtccaggcccgtcaactgggagtgagtgccgcgagcctggtccacctggcctgggcgcgattgttgggtcaggtctcgggccgtgacgacgtggtgtttggcaccgtgctgctgggccggatgcaaggcggcgacggcgccgatcgggcgctggggatgtttatcaacaccttgccgctgcgtgtgacgctgggcagtcgcagtgtctgtgaaagtgtcagagaggtgcatgacaaactgaccgcgttactcggccatgagcacgcctcgctggcactggcccagcgttgcagcggcgtggtcgcaccggcgccgttgttcagcgccttgctcaactatcgccacagcagcgttgccgtgaccgatgaggcgctgacggcctggaatggcatgcaatcgctgggcgatgaagaacgcaccaactatccgctgaccctgaacgtcgatgaccagggagaagacttcctgttgacggtccagaccgtgccgctgatcgatgccgcacggatctgcgcctatatgcagcaaaccctgaacagtctggtcgatgcactggaacaggcgccgcaaactccgttgcacgccatttcgatactgccgcgcaaagagcgtacccagttgctcgaacagtggaacgagcctggccagcactacgcgaatgacacaccgatccaccagcagttcgaagcccgtgccgcagcacggccc

SEQ ID NO: 41 - protein, 467 aa, Pseudomonas syringae pv. syringae strain B301D
QDIYALAPLQEGILYHHLAAAEGDPYLQYALFAFDSLERLHSFAQALQGVIARHDILRTAVLWERLDAAVQVVWREAPLCLDEQLLDPADGDIAEQLLKRLDPRNTRLDIRQAPMLRIGYAQDTVNNRWLGMLLFHHLVDDATSLRILSSEIEAHMLGQQASLPPSVPYRNYVAQAMLGVSREEHEAFFRDMLGDIDEPTLPFGLQDVQGDGRGIEEVRQLVDVDLSRRLRVQARQLGVSSASLYHLAWARVLGAVSGKEDVVFGTVLLGRLQGGAGSDRALGMFINTLPLRVTLGEQGVRSGLKATHARLSGLLAHEHASLVLAQRCSGVPASTPLFSALLNYRHVAAEVNQQTFDAWQGIDNLHGEERTNYPLTLSVNDDSVGFSLTVQAIACIDAQRVCAYVQTALENLVSALEQSGEVPLAGLSVLPAAEREQLVLGLNATELDYPHEQTIHGLFEAQVQRTP

SEQ ID NO: 42 - DNA, 1401 bp, Pseudomonas syringae pv. syringae strain B301D
caggacatctacgcgctggcgccgttgcaggaggggattttgtatcaccaccttgccgcggccgagggtgatccatacctgcaatacgcactgtttgcctttgacagcctcgagcgtctgcacagtttcgcccaggcgctgcaaggtgtcatagcgcggcacgacatcctgcgcaccgcggtgctttgggagcgcctcgatgcagcggtgcaagtggtctggcgtgaagccccactgtgcctggatgaacagctgctggacccggccgatggcgacatcgccgaacagttgctcaagcgcctggacccgcgtaacacgcggctggacatccgtcaggcgccgatgctgcgcatcggctatgcacaggacacggtgaacaaccgctggctgggcatgctgttgttccaccacctggtcgacgatgccacctcgctgcgcattctgagcagtgaaatcgaagcgcacatgctcggtcagcaagcctcgctgccgccgtccgtgccttatcgcaactacgtggcccaggccatgctcggcgtcagccgcgaggagcacgaggcgtttttccgcgacatgctgggtgacatcgatgaaccgacgctgccattcggtctgcaggacgtgcagggcgacggacgtggtatcgaagaagtgcgccagttggtcgacgtcgacctgagccgacgcctgcgcgtgcaggcccgccaactcggggtcagctccgccagcctgtatcacctggcctgggctcgtgtgctgggtgcggtatcgggcaaagaggacgtggtgttcggcaccgtgttgctcggtcgcctgcagggcggcgccgggtccgaccgtgcactgggaatgttcatcaataccttgccgctgcgtgtcacgctgggcgagcagggcgtgcgcagcggtctgaaagccactcacgcacgactcagcgggctgctggcccatgaacacgcctcgctggtactggcccaacgttgcagcggtgtacccgcttcgacgccactgttcagtgccttgcttaactaccggcatgtcgccgccgaggtcaatcagcagacgttcgatgcctggcagggcatcgacaacctgcacggcgaggaacgcaccaactatccgctgaccctttcagtaaacgatgacagtgtcggcttcagcctgacggtccaggccattgcctgcatcgacgcgcaacgcgtctgcgcctatgtgcagactgcgctggaaaatctagtcagcgcgctggaacagtccggcgaagtgccgttggccggtttgtcggttctgccggcagcagagcgtgagcaactggtgctcgggctcaacgctaccgagctggattatccgcatgagcaaaccatccatgggctgttcgaagcgcaggtgcagcgtactccg

SEQ ID NO: 43 - protein, 469 aa, Pseudomonas syringae pv. syringae strain B301D
QDIYPLAPLQEGILYHHLTAGQGDPYVLRALFGAESRERLDDFAQALQAVIERHDILRTAMVWEGLEEPVQVVLREATLAVDELTLDAAGGDIETQLHERYDPRHFRLDLRQAPLMRMVCAEDPANARWVAILLFHHIAIDHAALDLVKHEMQAFLLGEGHALPEAVPYRNYVAQVRLGVGADAHEGFFREMLADIDEPTLPFGVLETPGSDSLIEDVHLPVDDALSARLRTQARQLGVSAASLHHLAWARVVGALAGKSDVVFGTVLMGRMQGGDGADRALGMFINTLPLRVRLEGHAVREGVRTTHARLTALLGHEHASLAQAQRCSGVAAPAPLFNSLLNYRHSASESVASAEALQAWKGLQSLGSEEQSTYPITLSVDDLGQGFSLTVQALAQIGAQRIGEYMLTALGALVEALEQQPQTPLQRLQVLSAAERQQVLHDFNDTAREYPRNSSLQELFEQQVATQP

SEQ ID NO: 44 - DNA, 1407 bp, Pseudomonas syringae pv. syringae strain B301D
caggacatctacccgctggcgcccctgcaggaaggcattctctatcatcacctgacggccgggcagggcgacccctatgtattgcgggcattgttcggggccgaaagccgcgaacgtctcgacgacttcgcccaggcactgcaggcggtgatcgaacgccatgacatcctgcgcacggcgatggtctgggaaggcctcgaagaaccggtgcaagtggtgttgcgcgaagcaacgctggccgtggatgagctgacgctggacgcggctggcggcgacatcgaaacccagttgcacgaacgctatgatccccggcatttccgcctcgatttgcgccaggccccgttgatgcgcatggtgtgcgctgaagatccggccaacgcgcgctgggtcgctatcctgctgttccaccacatcgccattgaccacgctgcgctggacctggtgaagcacgagatgcaggcctttctgctcggcgaaggccatgccttgcccgaagcagtgccttatcgtaactatgtggcacaggtccgactgggtgtcggggctgatgctcatgaaggctttttccgcgagatgctggccgacatcgatgagccgacgctgccgttcggcgtgctcgaaacgccgggcagcgactcactgatcgaagatgtgcacctgcccgtcgatgacgcattgagcgcgcgtctgcggacccaggcccgacagcttggggtcagcgccgcgagtctgcatcacctcgcctgggcgcgagtggtgggtgcgctggcgggcaaatcggatgtggttttcggcacggtgctgatgggccggatgcagggcggcgacggcgccgaccgggcgttgggcatgttcatcaacaccttgccgttgcgggtcaggctcgaagggcatgcggtgcgtgaaggggtcagaaccacccatgcacggctgacggccttgctcggtcatgaacatgcgtcgctggcccaggcccagcgttgcagcggcgttgccgcaccggcgccgttgttcaattcgctgctcaattaccgtcacagtgccagcgaatcggtggcttcggccgaagcgctccaggcctggaaaggcttgcagagtctgggcagcgaggagcagagcacatacccgatcacgctgtcggtggatgacctgggccagggcttcagcctgacggttcaagccctggcgcagatcggtgcacagcgcatcggcgagtacatgttgacggcactcggcgcgctggtagaggcgctggagcagcagccgcaaaccccgttgcagcgtctgcaagtcctctctgcagccgaacgccagcaagtgctgcacgacttcaatgacacggcacgcgaatacccacgcaacagcagcctgcaggaacttttcgaacaacaagtggcgacgcagcct

SEQ ID NO: 45 - protein, 468 aa, Pseudomonas syringae pv. syringae strain B301D
QDIYALAPLQEGILYHHMAAEVGDPYVLQSQFAFDNRERLDAFVQALQMVIDRHDILRTGVVWDGLDSPVQVVWREARLHLEGLELDPADGEIGAQLHSRFDPRHYCLDMTQAPLMRLVYAEDPLNQRITAMLLFHHMALDHMAMDVVQHEMQAWLLGESETLSAPVPYRNYVAQARLGVSQADHEVFFRDMLGDIDEPTLPFGLQDVQGDGRDIEEAALAVDSQLNLRLRAQARQQGVSAASLVHLAWAQVLGKVSDRRDVVFGTVLLGRMQAGEGADRALGMFINTLPLRVAVGGQGVRAGVKATHAQLTALLAHEHASLALAQRCSGVAAPTPLFSALLNYRHSAVGSVSERAVQAWQGIHALSSEERTNYPLTLNVDDLGDGFKLAALATNAIGAQRVCGYMHTALEHLVDALELMPQASLQGLSILPAVEREQLLVGFNDTALDYPHEQTIHGMFEAQVERTP

SEQ ID NO: 46 - DNA, 1404 bp, Pseudomonas syringae pv. syringae strain B301D
caggatatctacgcgctggcgccgttgcaggaaggcattctttatcaccacatggcagccgaggtcggcgacccctatgtgttgcagagccagttcgcctttgataaccgcgagcgcctggacgccttcgttcaggctttgcagatggttatcgaccgtcatgacattctgcgtaccggggtggtctgggatggtctggactcaccggtgcaagtggtctggcgcgaagcgcggttgcaccttgaagggcttgagcttgacccggcagacggtgagatcggcgcgcaactgcatagccgcttcgacccgcgtcactactgtctcgatatgactcaggcgccactgatgcgcctggtctacgccgaggatccgctcaatcagcgaatcaccgcgatgctgctgttccatcatatggccctggatcatatggccatggacgtggtgcagcatgaaatgcaagcctggctgctcggcgagagcgaaaccctgtcggcgccagtgccataccgcaactacgtggctcaggcgcggctgggtgtcagccaggctgatcatgaagtgtttttccgcgacatgctgggtgatatcgacgagccgacgctgccgttcggcctgcaggatgtgcagggcgacgggcgtgatatagaagaagccgcccttgccgtggatagccagctgaacctgcgcctgcgtgctcaggcccgacaacaaggggtgagtgccgccagtctggtgcacctggcctgggcccaggtgctgggcaaggtttccgatcgtcgcgatgtggtgttcggcaccgtactgctgggccgtatgcaggccggcgaaggcgccgaccgggcactggggatgttcatcaacaccttgccgctgcgggtcgccgtgggcgggcagggtgtacgcgccggggtcaaggcgacacacgcgcaactcactgcgttgctcgctcacgaacatgcatcgctggcgctggctcagcgttgcagcggggttgccgcaccgacgccactgttcagcgcactgctcaactaccgccacagtgcggttggcagcgtgtccgaacgtgccgtacaggcctggcagggtattcatgccctgagcagtgaagagcgcaccaactatccattgacgttgaacgtcgatgatttgggtgacggcttcaaactggctgcgctggcgacgaacgcgatcggtgcgcagcgcgtgtgtggctacatgcacacggctctggagcatctggtggacgcgcttgagctaatgccacaagcgtcgttacaaggcttgtcgatcctgccggctgtcgaacgtgagcagttgctggtgggtttcaacgacaccgcgctggattacccgcatgaacagaccattcacggtatgttcgaagcgcaggtcgagcgcacgcct

SEQ ID NO: 47 - protein, 468 aa, Pseudomonas syringae pv. syringae strain B301D
QDIYPLAPLQEGILYHHIAAEQGDPYVLQAHFAFDDRACLDAFVQALQTVIDRHDILRTGVVWKGLDSPVQVVWRHAQLPLEMLQLDPAAGDVSAQLHDRFDPRHHRLDVTQAPLMRLAYAEDPLNQRICAMLLFHHMVLDNMAMAVVQHEMQAWLLGEAQNLSTPVPYRNYVAQARLGVSQADHEAFFRDMLGDVDEPTLPFGLINVQGDGLDIEEANLALAPRLNLRLRAQARQQGVSTASLLHLAWAQVLSSVANRQDVVFGTVLMGRMQAGEGAERALGMFINTLPLRVSVGGKSVRDGVKATHERLTALLGHEHASLALAQRCSGIPAPTPLFSSLLNYRHSAPGSVSERATQAWQGIHTLNSEERTNYPLTLNVDDSGEGFNLNVLVTGAVGAKRVCAYMQTVLEHLVDALEQSPSAALDSLSILPAGERAQLLVAFNDTAMAYPQQQTIHGLFEAQVERTP

SEQ ID NO: 48 - DNA, 1404 bp, Pseudomonas syringae pv. syringae strain B301D
caggacatctacccgctggcaccgttgcaggaaggcattctctatcaccacatcgccgccgagcagggcgatccctatgtgctgcaggcgcatttcgcctttgatgaccgcgcgtgcctggacgccttcgtccaggctttgcagacggttatcgatcgtcacgacattctgcgcaccggtgtcgtctggaaaggtctggactcacccgtgcaggtggtctggcgccacgcacaattgcctcttgagatgttgcaactcgacccggcagccggtgatgtcagcgcgcaactgcacgaccgcttcgacccgcgtcaccaccgccttgatgtcactcaggcaccgctgatgcgcctggcctatgccgaagacccgctgaatcagcgcatctgcgcgatgctgctgttccatcacatggtcctcgataacatggcgatggccgtggtacagcatgaaatgcaggcctggttgctgggcgaagctcaaaacctgtcgacaccggtgccataccgcaattatgtggcccaggcacgcctgggcgtcagccaggccgatcacgaagcgtttttccgcgacatgctcggtgatgtggacgagccgacgctgccgttcggtctgatcaatgtgcagggcgatggccttgatatcgaggaagccaaccttgcgctggcccctcggttgaacctgagactgcgcgctcaggcccgacaacagggggtgagcaccgccagtctgctgcacctggcctgggctcaggtattgagctcggttgcaaacaggcaggacgtggtgttcggcaccgtgctaatgggccggatgcaggccggggaaggcgccgagcgggcattggggatgttcatcaacaccttgccgctgcgggtttcggtgggcgggaaaagcgtgcgggacggggtcaaggccactcatgaacgtctgactgccttgctgggtcatgagcatgcttcactggcgctggctcagcgttgcagcggcattcccgcgccaacgccactgttcagttcgctgctcaactatcgccatagtgcgcctggcagcgtatccgagcgggcgacccaggcctggcagggtattcatacgctgaacagcgaagaacgcaccaactatccattgaccttgaacgtcgatgactccggtgaaggcttcaacctgaacgtactggtcaccggcgccgtgggtgccaaacgggtctgcgcttacatgcagaccgtgctggaacatctggtggatgcgcttgagcagtcaccgtctgcggccctcgacagcctgtcgatcctgcctgctggcgaacgtgcgcaattgctggtggcgttcaacgacacagcaatggcttacccgcagcagcagaccattcatggcttgttcgaagcacaggtcgagcgcaccccg

SEQ ID NO: 49 - protein, 469 aa, Pseudomonas syringae pv. syringae strain B301D
QDIYPLAPLQAGILYHHISAEQGDPYVLQAQFAFDSRTHLDTFAQTLQTVINRHDILRTSMHWESLDEPLQVVWRHVELSVEEVQLNPGLGDISRQLQERLDPRQIRLDIRQAPLMRLVCALDTVNQRWLATLMFHHMILDHTALDQVRYEMQVCLLGQADQLGDSIPYRNYVAQARLGLNESDHELFFKEMLGDIDEPTLPFDLHDVQGDGNAIDQARLMLDSGLSQRLRVQARQLGVSAASLLHLAFAQMLGRLSGREHVVFGTVLMGRMQSGEGAERALGMFINTLPLRVDLGEQAVRDGVKVTHRRLTGLLGHEHASLALAQRCSGVVAPAPLFSALLNYRHSSVAVTDEALTAWNGMQSLALDDEERTNYPLTVNVDDMGEGFLFTALVAASIGAQRLCDYLQLAAEGLVDALEQAPQTPLHAISILPLNERKQLLEQWNEPGQHYANGIPIHQQFEARAAARP

SEQ ID NO: 50 - DNA, 1407 bp, Pseudomonas syringae pv. syringae strain B301D
caggacatctacccgctggcccccttgcaggcgggcattctctatcaccacatcagcgccgaacagggcgacccctatgtattgcaggcgcagttcgcctttgacagccgcactcacctggatacctttgctcaaacgctgcaaacggtcatcaaccgccatgacatcctgcgcacctccatgcactgggaaagcctcgacgagccgttgcaggtggtctggcgccatgtcgagctcagcgtagaagaagttcagctcaaccctggccttggtgatatatcccgtcagttgcaagagcgtctggacccacgccagatacgcctggacatccgtcaggcccccttgatgcgtctggtgtgtgcgttggacaccgtcaaccagcgctggctggcgaccttgatgtttcaccacatgatccttgaccacaccgcgctggatcaggtgcgctatgagatgcaggtctgcctgctcggtcaggccgatcagcttggcgactccattccttaccgcaactatgtcgctcaggcccgactgggccttaacgaaagcgaccacgagctgttcttcaaggaaatgcttggcgacatcgacgagccgacgctgccgttcgacctgcacgatgtacaaggcgacggtaacgctatcgatcaggcccgtctgatgctggacagcggtttgagccagcgtctgagagttcaggcgcggcagcttggggtcagcgcggcgagcctgctccatctggcgttcgcgcaaatgctgggtcgcctcagcggacgtgaacacgtagtgttcggcaccgtattgatgggccggatgcagagcggcgagggcgccgaacgagccctcggcatgttcatcaataccttgccgctgcgggtggacctgggcgaacaggccgtgcgtgacggagtcaaggtcacgcatcggcggttgaccgggctactcgggcatgagcatgcctcactggcgctggcccagcgttgcagcggcgtagttgcaccggcgccgttgttcagcgccttgctcaactatcgtcacagcagtgttgccgtgaccgatgaagcgcttaccgcctggaatggcatgcagtcgctggcgctggacgacgaagagcgcaccaactacccattgaccgtcaatgtcgacgatatgggcgaaggcttcctgttcactgcgctggtggcggcgagcataggcgcgcagcgtctttgcgactatctgcagttggcagcagaagggctggtcgatgcgctggaacaggcgccgcaaactccgttgcacgccatttcgatactgccgctcaacgagcgtaagcagttgctcgaacagtggaacgagcctggccagcactacgcgaatggcatacccatccaccagcaattcgaagcccgtgccgcagcacggccc

SEQ ID NO: 51 - protein, 466 aa, Pseudomonas syringae pv. syringae strain B301D
QDIYALAPLQEGILYHHLAAAEGDPYLQYALFAFDNIERLYSFAEALQGVIARHDILRTAVLWERLDSPVQVVWREATLGLDEQVLDPADGDITEQLLKRLDPRHTRLDIRQAPMLRIGYAQDAANNRWLGMLLFHHLVDDATSLRILSSEIEAHMLGQQASLPPSVPYRNYVAQAMLGVSREEHEAFFRDMLGDIDEPTLPFGLQDVQGDGRGIEEVRQLVDVDLSRRLRVQARQLGVSSASLYHLAWARVLGAVSGKEDVVFGTVLLGRLQGGAGSDRALGMFINTLPLRVTLGEQGVRSGLKATHARLSGLLAHEHASLVLAQRCSGVPASTPLFSSLLNFRHTGDLDASDQALAAWEGIQALHGEERTNYPLTLCVDDLGEGFNLTVMAEGQIGAKRVCTYMHCVLENLVQALEQTPDAALGALNILPASERQQLLESWNTPHALQADDALIHRTFEAWVVA

SEQ ID NO: 52 - DNA, 1398 bp, Pseudomonas syringae pv. syringae strain B301D
caggacatctacgcgctggcgccattgcaggaggggattttgtatcaccaccttgctgcggccgaaggtgatccataccttcaatacgcactgtttgcctttgacaatatcgagcgtctgtacagcttcgccgaggccctgcaaggcgtgatcgcccggcacgatatcctgcgcaccgcggtgctttgggagcgcctcgattccccggtgcaagtggtctggcgtgaagcgactctgggtctggatgaacaggtgctggacccggccgatggcgatatcaccgaacaattgctcaagcgcctggaccctcgtcatacgcgcctggacatccgtcaggcgccgatgctgcgcatcggctacgcacaggacgcggcgaacaaccgctggctgggcatgctgctgttccatcacctggtcgacgatgccacctcgctgcgcattctgagcagcgaaatcgaagcgcacatgctcggtcagcaagcctcgctgccgccgtccgtgccttatcgcaactacgtagcccaggccatgctcggcgttagccgcgaggagcacgaggcgtttttccgcgacatgctgggtgacatcgatgaaccgacgctgccgttcggtctgcaggacgtgcagggcgacggacgtggtatcgaagaagtgcgccagttggtcgacgtcgacctgagccgacgcctgcgcgtgcaggcccgccaactcggggtcagctccgccagcctgtatcacctggcctgggcccgtgtgctgggtgcggtatcgggcaaagaggacgtggtgttcggcaccgtgttgctcggtcgcctgcaaggcggcgccgggtccgatcgggcgctggggatgttcatcaacaccttgccgctgcgtgtcacgctgggcgagcagggggtgcgcagcggtctgaaagccactcatgcacgactcagcgggctgctggcccacgaacacgcctcgctggtactggcccagcgttgcagtggcgtaccggcttcgacaccattgttcagctcgctgctcaacttccgccataccggtgatctcgacgcgagcgaccaggcgctggccgcctgggaaggtattcaggcgctgcatggcgaagaacgcaccaactacccgctgaccttatgcgtggatgacctgggcgaaggtttcaacctgaccgtcatggccgaagggcagatcggtgccaagcgtgtgtgtacctacatgcactgtgtgctggaaaatctggtgcaagcactggagcagaccccggacgcagcgctgggagcactgaatatcctgcctgccagcgagcgccagcaactgctcgaaagctggaacaccccgcatgcgctgcaggccgacgacgccttgattcaccgtaccttcgaagcctgggtggtggcg

SEQ ID NO: 53 - protein, 468 aa, Pseudomonas syringae pv. syringae strain B301D
QDIYALAPLQEGILYHHLAADEGDPYVLQMLFAFDDRECLAGFAAALQSVVERHDILRTSVVWEGLERPVQVVWRNAKLSLEEVTIDPLDGDVLTRLRERFDPRHYRLDIGQAPLMRIAYAEDNTHQRLVGMLLFHHLALDHTSLEVVVEEMQASLQGQIEQLPAPVPYRNHVAQARLGISQAEHEAFFRDMLGDIDEPTLAYGIQDVQGDGSGIEEVHQVLDSQLSSRIRSIARQLGVSAASLAHLAWAQVAGRVSGREEVVFGTVLMGRMQGGNGADRALGMFINTLPLRISVGSQSALAAVKVTHQRLSALLGHEHASLSLAQRCSGVPSSLPLFSTLLNYRHSNGGAASSETLSAWQGIQTLSMEERTNYPLCLNVDDLGDDFMLTIQAVQQISATRIGEYMQVALRSLVDALEHTPQAALNSLSILPDDERELLLTGFNDTAHPYPRDVLIHQLIEQQAAQRP

SEQ ID NO: 54 - DNA, 1404 bp, Pseudomonas syringae pv. syringae strain B301D
caggacatctacgctctggcccccttgcaggaaggcattctctatcaccacctggccgcggatgaaggtgacccctacgtcttgcagatgctcttcgccttcgatgatcgcgagtgcctggccggtttcgccgcggccttgcagagcgtggtcgaacgccacgatatcctgcgcaccagtgtggtctgggaagggcttgagcggccggtacaggtggtatggcgcaacgcgaaactgagcctggaagaagtgaccatcgatccgctcgacggtgacgtgctgacacgactgcgcgagcgttttgacccgcgtcactatcgcctggacatcggccaggcgccgctgatgcgcatcgcctacgccgaggacaacacccaccagcgtctggtcggcatgttgctgttccaccatctggcgctggatcacacctcgctggaagtggtggtggaggaaatgcaggccagcctgcaggggcagatcgagcaattgccggccccggtgccttatcgcaaccatgtggcccaggcgcgtctgggtatcagccaggccgagcacgaagcgtttttccgcgacatgctcggcgacatcgacgaaccgaccctggcttacggcatacaggacgtgcagggcgatggcagtggtatcgaggaagtgcatcaggtgctcgacagccagctgagcagccgtatacgcagtattgcgcggcagttgggagttagcgctgccagtctggcgcacctggcatgggctcaggtggcaggtcgggtttcgggccgtgaagaagtggtattcggtaccgtactgatgggccggatgcagggcggcaatggtgccgaccgggcgctgggcatgttcatcaacaccttgccgctgcgcatcagcgtcggcagccagagcgctctggcggcggtcaaggtcactcaccagcgcctgtcggcgctgctggggcatgagcatgcctcactgtcgctggcccagcgctgcagcggcgtgccgagttcgctgccgctgttcagcaccttgctcaactaccgccacagcaacggtggtgcggcctcgagcgaaacattgtcagcctggcagggcattcagaccctgagtatggaggaacgcaccaactatccgttgtgcctgaacgtcgatgacctgggcgatgacttcatgctcaccattcaggccgtccagcaaatcagtgccacgcgcatcggcgagtacatgcaggttgcactgcgcagcctggtggacgcactggaacacacgccgcaggcagcgctgaacagcctgtcgatactgccggacgatgagcgcgagctgttgctgacgggcttcaacgacacagcccacccttatccgcgtgatgtactgatccaccaactgatcgaacaacaggccgcccagcgtccg

SEQ ID NO: 55 - protein, 470 aa, Pseudomonas syringae pv. syringae strain B301D
QDIYPLAPLQAGILYHHLAAEIGDPYVLQTQFAFDNRERLNAFVQALQMVIDRHDILRTSVVWDGLDSPVQVVWRQAQLHLDALELDPADGDIGAQLHSRFDPRHYRLDIGQAPLMRVAYAEDSLNQRICAMLLFHHMALDHVALEVVKHEMQAWLAGEADTLAASVPVPYRNYVAQARLGVSEAQHEAFFRDMLGDIDEPTLPFGLQDVQGEGRDIEEASLALDPQMSLRLRAQARQQGVSAASLVHLAWAQVLGKVSNRQDVVFGTVLMGRMQGGEGAERALGMFINTLPLRVSVGEQGVRDGVKATHKRLTALLGHEHASLALAQRCSGVAAPAPLFSALLNYRHSGVGSVSNQAMQAWQGIAVLSGEERTNYPLTLNVDDLGEGFSLTALVVSSIGAQRVCGYMHTALENLLMALEQTPETSLQGLSILPVVEREQLLVAFNDTALDYPQQQTIHGMFEAQVERTP

SEQ ID NO: 56 - DNA, 1410 bp, Pseudomonas syringae pv. syringae stain B301D
caggacatctacccgctggcgccgttgcaggcaggcattctttatcaccatctggccgccgaaatcggtgacccctatgtgttacagacgcagttcgcctttgataaccgcgagcgcctgaacgccttcgttcaggccttgcagatggtcatcgaccgtcacgacattctgcgcaccagcgtggtatgggacggtctggactcgccggtgcaagtggtctggcgtcaggcacagttgcaccttgacgcacttgagcttgatccggcagacggcgatatcggtgcgcaactgcacagccgctttgatccgcgtcattaccgactggacatcggccaggcacctttgatgcgtgtggcctatgccgaagactcgctcaaccagcgcatctgcgcgatgctgctgttccatcacatggcgctggaccacgtcgcgctggaagtggtgaagcatgaaatgcaggcatggctggcgggcgaggccgacactttggcggcgtcggtacccgtgccgtatcgcaactatgttgctcaggcacgcctgggtgtcagcgaggcacagcacgaagcgttcttccgcgacatgctcggcgatatcgacgagccgacgctgccgttcggtttgcaggatgtgcagggtgaaggtcgcgacatcgaggaagccagcctggcgctggacccccaaatgagcctgcgcctgcgtgctcaagcccgccagcaaggggtgagtgccgcgagcctggtgcatctggcctgggctcaggtgctgggcaaggtgtcaaacaggcaggatgtggtgttcggcactgtactgatgggccggatgcagggtggcgaaggtgccgagcgagccttggggatgttcatcaacaccttgccgctgcgggtttcagtgggtgagcagggtgtgcgtgacggggtcaaggccactcataagcgcctgacggccttgctgggccatgaacatgcctcactggcgttggcccagcgttgcagcggggttgccgcaccggcgccgctgttcagtgcgctgctaaactatcgccacagtggggtcggcagtgtgtccaaccaggccatgcaggcctggcagggcatcgcggtcctcagcggtgaagaacgcaccaactacccactgaccttgaacgtcgacgatctgggcgaagggttcagcctgacggcgttggtggtttcgtcaatcggcgcgcaacgggtgtgcggctacatgcacacggcgctggaaaacctgttgatggcgctggagcagacgccagaaacgtcattgcaaggtttgtcgatcctgccagttgtcgagcgcgagcagttgctggtggcgttcaacgacaccgcactggattacccgcagcagcagaccattcacgggatgttcgaagcccaggtcgagcgcacgccg

SEQ ID NO: 57 - protein, 467 aa, Pseudomonas syringae pv. syringae strain B301D
QDIYPLSPLQEGILYHHLSAERGDPYLLQSLFAFDSQEHVDDFARALQFVIQRHDILRTALVWEGLDEPMQVVWRQALLMRESFEPDPQGGDVATQLHQRFDSRHQRLDIRRAPMMRLVHAWDEPNQRWLGWLQFHHLVMDHTTLEVVRHEMQACLLGQGAQLGLTVPYRNYIAQVRLGMSQEEHEAFFRDMLSDVDEATLPFGLQEVQGDGHDIEEGCLALDLELSRRLRAQSRQLGVSAASLVHLAWAQLLSKASGRDDVVFGTVLLGRMQGGEGSERALGVFINTLPLRVSLGDQGVRDGVKATHRRLTALLGHEHASLALAQRCSGVVAPAPLFSALLNYRHTSVAVTDETLTAWNGMHALALGDEERTNYPLTLNVDDRGEDFLLTVQTVPLIDGARICAYMQQTLSNLVNALEQAPQTPLHAIAILPDSEREQLLEQWKQPGTAYTSETPIHLQFEARAAQ

SEQ ID NO: 58 - DNA, 1401 bp, Pseudomonas syringae pv. syringae strain B301D
caggacatctacccgctgtcgccgttgcaggaaggcatcctctatcaccacctcagcgctgagcgtggagatccgtacttgctgcaatcgctgttcgccttcgacagccaggaacatgtcgacgactttgcccgcgccttgcagttcgtgatccagcgccatgacattctgcgcaccgcactggtctgggaggggctggacgaaccgatgcaagtggtctggcgtcaggcgttgctgatgcgtgaaagctttgagccggacccgcaaggcggtgacgtcgcgacacagttgcaccagcgtttcgactcccgccaccagcgtctggacattcgtcgggcaccgatgatgcgtcttgtacatgcctgggatgagccaaaccagcgctggctgggctggctgcagttccatcatctggtcatggaccataccacgctggaagtagtgcgccatgagatgcaggcctgcctgctggggcagggtgctcaactgggcttgacggtgccatatcgcaattacatcgctcaggtccgcctgggcatgagtcaggaggaacatgaggcgttcttccgcgacatgttgagtgacgttgacgaggcgacgctgccgtttggtttgcaagaggtgcaaggtgacggtcatgacatcgaagaaggctgtctggcgttggatctggagctcagccgtcgtttgcgcgctcagtcccgtcaactgggcgtaagtgccgcgagcctggtccacctggcctgggcgcagctgttgagcaaggcctcggggcgtgacgacgtagtgttcggcaccgtgctgctgggccggatgcaaggtggtgaaggctcagaacgggcgctgggcgtgtttatcaacaccttgccgctgcgtgtgagtctgggcgaccaaggcgtgcgtgacggggtcaaggccacgcaccgacggctgaccgcattgctcgggcatgaacacgcgtctctggcgctggcccagcgttgcagcggcgtggtcgcaccggcgccgttgttcagcgcgctgctcaactatcgccacaccagcgttgctgtgaccgatgagacgctgacggcctggaatggcatgcacgccctggcgctgggcgatgaagaacgcaccaactatccattgaccctcaacgtcgatgaccggggcgaagacttcctgttgaccgtacagaccgtaccgctgatcgacggcgcacgtatctgcgcctacatgcagcaaaccttgagcaatctggtcaacgcgctggagcaggcaccgcagaccccgttgcacgccattgcgatactgccggacagcgagcgcgagcagttgcttgaacagtggaagcagcccggcactgcctatacgagcgagacaccgattcatctgcaattcgaagcccgcgccgcgcaa

SEQ ID NO: 59 - protein, 467 aa, Pseudomonas syringae pv. syringae strain B301D
QDIYALAPLQEGILYHHLAAVEGDPYLQYALFAFDSLERLQGFAQALQGVIARHDILRTAVLWERLDAPVQVVWREAPLGLDELILDPADGDIAEQLLERLDPRHTRLDIRQAPMLRIGYAHDAENDRWLGMLLFHHLVDDATSLRILTSEIESYMLGQQASLPPSVPYRNYVAQAMLGVSREEHEAFFRDMLGDIDEPTLPFGLLDVQGDGRGIEEVHQPVDMDLSRRLRLQARQFGVSSASLYHLAWARVLGAVSGKEEVVFGTVLLGRLQGGAGSDRALGMFINTLPLRVTLGEQGVRSGLKATHARLSGLLAHEHASLVLAQRCSGVAASTPLFSALLNYRHVAGQANQQTLDAWQGIKSLRGEERTNYPLTLSVNDEGTGFSLTIQASACIDAQSICAYVQTTLENVVSALEQSGEVPLAGLSVLSAAEREHLVYGLNATALDYPQQQTIHGMFEAQVERTP

SEQ ID NO: 60 - DNA, 1401 bp, Pseudomonas syringae pv. syringae strain B301D
caggatatctacgccctggcgccgctgcaggaagggattttgtatcaccaccttgccgcggtcgaaggtgacccgtatttgcaatacgcgctgtttgcctttgacagccttgagcgtctgcagggtttcgcccaggcactgcaaggcgtgatcgcccggcacgatatcctgcgcaccgccgtgctttgggagcgtctcgacgctccggtgcaggtggtctggcgtgaagcacctttgggcctggatgagctgatacttgacccggccgatggcgatatcgccgaacaactgctggaacgtctggaccctcgccacacgcgtctggacattcgtcaggcaccgatgctacgcatcggttatgcacatgacgcggagaacgaccgctggctgggcatgctgctgttccatcacctggtggacgacgccacatcgctgcgcattctcaccagcgaaatcgagtcgtacatgctcgggcagcaggcctctctgccaccgtccgtgccttaccgcaattacgtggcgcaagccatgctcggcgtgagccgcgaggagcacgaagcgtttttccgcgacatgctcggtgacatcgatgagccgacgctgccgttcggcctgctggacgtgcagggcgacggtcgcggtatcgaggaagtgcaccagccggtcgatatggacctgagccgccgcctgcgcttgcaggcccggcagttcggggtaagctccgccagtctgtatcacctggcatgggcacgtgttctgggcgctgtgtcaggcaaagaggaggtggtgttcggcaccgtattgctcggccgcctgcagggtggtgccgggtctgaccgggcgctggggatgttcatcaacaccttgccgttgcgtgtcacgctgggcgagcagggcgtgcgcagcggtcttaaggctacccacgccaggctcagcgggctgcttgcccatgaacacgcctcgctggtactggcacagcgttgcagtggcgtggccgcttcaacgccgctgttcagcgccttgctcaactaccggcatgtcgccggtcaggccaaccagcagacactggacgcctggcaaggcattaaaagcctgcgtggcgaagaacgcaccaactatccgctgaccctttcagtaaacgatgagggaacgggcttcagtttgacgattcaagccagtgcctgcatcgatgcgcaaagcatctgcgcgtatgtgcagaccactctggagaatgtggtcagcgcactggagcagtccggcgaggtgccgctggcaggcttgtcggttctgtcggcggcggagcgtgagcatctggtgtatggcctgaacgctaccgcgctggattacccgcagcagcagaccatccatgggatgttcgaagcccaggtcgagcgcacgccg

SEQ ID NO: 61 - protein, 468 aa, Ralstonia solanacearum strain GMI1000
EDIYPLAPLQEGILYHHLAAQQGDPYVLHAMFGMADRERVNAFAQALQAVIDRHPILRTAVVWQGLDEPMQVVWRHAELVVEEVALESGADPAGQLRSMFDPRRRPLLAIGQAPLVKLVYAADAATGRCVAMLLFHHLVLDHLALERVRQEMQAHLSGQAAWLPAVVPYRDYVAQAKQRVSQAAHEAFFREMLGDVEAPTLPFGLQDVQGDGGAIEEVTLRLDAALSARLRHQGRQAGVSAASLHHLAWARVVAVLSGRDDVVFGTVLLGRLQGGRSAERALGLFINTLPLRVAVHGQDVLAGTRAVHARLSALLGHEHAPLALAQRCSGVAAPQPLFSALLNYRHSAAPAASAATAPTWSGIDVLEMKERTNYPLTVSVDDLGDGFELNVQSAAGIDPERIAGYLETALVSLTEALERGGREPLHSLTVLPVAERQLLLARWNETAADSARQASLSGLFEAQVVRAP

SEQ ID NO: 62 - DNA, 1404 bp, Ralstonia solanacearum strain GMI1000
gaggacatctatccgctggcgccgctgcaggaggggattctctatcaccacctggccgcgcagcagggggatccctatgtcctgcacgcgatgttcggcatggcggaccgcgagcgcgtcaacgccttcgcgcaggccctgcaggcggtgatcgatcggcacccgatcctgcgcacggccgtggtgtggcagggcttggacgagccgatgcaggtggtctggcggcatgccgagctggtcgtggaagaggtcgcgctggagtccggtgctgatccggccgggcagcttcggtcgatgttcgacccccgccgccgcccgctgctggcgatcggccaggcgccgctggtgaaactggtctatgccgcggacgcggcgacgggccgctgcgtggcgatgctgctgttccaccacctggtgttggaccacctggcattggagcgggtgcgacaggaaatgcaggcccacctgtcggggcaggccgcgtggctgcccgccgtggtgccctatcgcgactacgtggcccaggcgaagcagcgcgtcagccaggccgcgcacgaggcctttttccgcgagatgctgggcgacgtggaagcgcccaccttgcccttcgggctgcaggatgtgcagggcgacggtggcgccatcgaggaagtcaccttgcgtctggacgccgcgctgagcgcgcgtctgcggcatcagggacggcaagcgggcgtcagcgcggcgagcctgcaccacctggcctgggcccgggtcgtggcggtgctgagcggccgggacgacgtggtgttcggcacggtgctgctgggccggctgcagggcgggcgcagtgccgaacgcgccctgggcctgttcatcaatacgctgccgctgcgggtggcggtgcacgggcaagacgtactggccggcaccagggccgtgcacgcgcgactgagcgcgctgctgggccacgaacacgcgccgctggcgctggcacagcgctgcagtggcgtggcggcgccgcagcccctgttcagcgcgctgttgaactatcgccatagcgcggcgcccgctgcgtcggcagcgaccgcgcctacctggtccggcatcgacgtgctggaaatgaaggagcgcaccaactatccgctgacggtctcggtggacgacctgggcgacggcttcgagttgaacgtgcagagcgcggcgggcatcgatccggaacggatcgccgggtatctggagacggcattggtcagcctgaccgaagcgctggaacgcggcggccgggagccgttgcacagcctgaccgtcttgccggtagccgagcgtcagctgctcctggcgcgatggaacgagacggcggccgactctgcccggcaggcatcgttgtcgggcctgtttgaggcgcaggttgtccgcgcgccc

SEQ ID NO: 63 - protein, 469 aa, Ralstonia solanacearum strain GMI1000
EDIYPLAPLQEGILYHHLMAKQRDPYLLFAMFRMDSRARLEAFAQGLQSLIARHTILRTAVIWDGLDEPMQVVWRQAALERQQMRLDAADGDIATQLKQRFDQGLHGLDLRQAPLMRLVFAEDAARGGWVAMLVFHHMIDDATSMKWLHTELEARLANEARHLPRAIPFRNYVARTRQAIAGNAHEAFFREMLADVVEPTLPFGLQDARGDDLAIGQATRRLSGPLSRRLRQQARLLKVSAASLHHLAWARVVSATSGRDDVVFGTVLMGRSQGGRGAEHTVGMFINTLPLRVLLDDRMVSAGARDTHVRLPALMGHEYAPLAEAQRCSGVAAPQPLFGALLNYRQNMPQPEPAGQVSAAWTGIDVLGMDERTNYPLTAVVDDLGEDFGLIVQSVPGMDAERIVGYLETALASLVASLERGGRESLRSLTVLPEAERHQQIDGWNRTQAAYASASTLPGLVEAQAARTP

SEQ ID NO: 64 - DNA, 1407 bp, Ralstonia solanacearum strain GMI1000
gaggatatctatccgttggcaccgttgcaggaaggcatcctctaccaccatctgatggcaaagcagcgcgatccttacctgcttttcgccatgttccgcatggacagccgcgcgcgcctggaggcgttcgcgcaaggtctgcaaagcctgatcgcgaggcacaccatcctgcgaacggccgtgatctgggacggcctggacgagccgatgcaagtggtctggcgacaagccgcgctggaacgccagcagatgcggctcgatgctgccgacggcgacatcgcaacgcagctgaagcagcgcttcgaccagggactccacggcctcgaccttcggcaggcgccgctgatgcggctggtcttcgcggaagacgcggccagggggggctgggtcgccatgctggtgttccaccacatgatcgacgatgccacctcgatgaaatggctgcacaccgagctcgaagcccgtctggcgaacgaggctcggcacttgccccgcgcaatccccttccgaaactatgtggcgcgcacccggcaggccatcgccggcaacgcgcacgaagcgtttttccgcgagatgctggccgatgtggtggagccgaccctcccgttcggcctgcaggatgctcgcggagatgacctcgccatcgggcaggcgacacgccggttgagcggccccctcagccgccggctgcggcagcaggcacggctgctgaaggtgagtgcggccagcctgcatcacctggcctgggcccgggtcgtgagcgcgacgagcggccgcgacgacgtggtgttcggtacggtgctgatgggccgttcgcaaggcggtcgcggtgccgagcacacggtgggcatgttcatcaatacgctgccgctgcgcgtgctgctggacgaccggatggtgtccgccggcgccagggacacgcacgtccggctgcccgcgctgatgggccacgagtatgcgccgctggccgaggcccagcgctgtagcggcgtggcggcgccgcagcccttgttcggcgcgctgctgaactatcgccagaacatgccgcagcccgaacccgccggccaggtgtccgcggcatggacgggcatcgatgtcctgggcatggacgagcgcaccaactatccgctgacagccgtcgtggacgatctcggagaagacttcgggctcatcgtgcaaagcgtgccgggcatggatgccgagcggatcgtcgggtatctggagaccgcgctcgccagtctggtcgcgtcccttgagcgcggcgggcgcgaatcgctgcgcagcttgactgtgctgcccgaagcggaacgccatcagcagatcgatggttggaaccggacgcaggcggcgtacgcgagtgcatcgacgctgcccggcctggtggaggcccaggcggcccgcacgccg

SEQ ID NO: 65 - protein, 469 aa, Streptomyces aizunensis strain NRRL B-11277
ADIYPLAPLQEGLLFHHLMAEGGEDAYLMPVALEMDSAERVAEFARAFQLVVDRHDILRTSFVWEGLREPVQVVWREAVLPVTEVVLDPEATDPAAALQAAVGLSMTPSRAPLISVHFAALPDGDRHLVLLRLHHLVQDHTALEVLLHEVQMFLAGRGGELEPSLPFRDFVAQVRGGMERGEHERYFAELLGDVTEPTAPFGLIDVRGDGTEVERAVVPFAPEVVEELRAVARRAGTSAATVLHVAWARVLAAVSGHSDVVFGTVLFGRMNAGAGADRVAGLYMNTLPVRVRTGELGALEAVTAMRGQLAGLLEHEHASLALAQQASGLTGNSPVFTSLLNYRHNTGRDDAPEGGDAPEGIRVLSSRERTSYPLGVSVDDYGDSMSLAVDAVAPIDAVAVGTLLRTAVEGLVPLIGQALDGGPDTALAAVDVLAPEDLNRLLVEWNDTVAEVEPSTLPALFAAQVERTP

SEQ ID NO: 66 - DNA, 1407 bp, Streptomyces aizunensis strain NRRL B-11277
gccgacatctacccgctggccccgctccaggagggtctgctcttccaccacctgatggcggagggcggcgaggacgcctacctgatgccggtggccctggaaatggactcggcggagcgggtggcggagttcgcccgcgcgttccagctcgtggtggaccgccacgacatcctgcggacctcgttcgtgtgggagggcctgcgcgagccggtccaggtggtctggcgcgaagccgtactgccggtgaccgaggtggtcctcgaccccgaggccaccgacccggccgccgccctacaggccgccgtcggcctgtcgatgactccgagccgcgcaccgctgatcagcgtgcacttcgccgccctgccggacggtgaccggcacctggtgctgctgcgcctgcaccacctggtccaggaccacacggctcttgaggtgctgctgcacgaggtgcagatgttcctcgccgggcgcggtggcgagctggagccctcgctgccgttccgtgacttcgtcgcgcaggtgcgcggcggcatggaacgcggcgagcacgagcggtacttcgccgagctgctgggcgacgtcacggagccgacggctcccttcgggctgatcgacgtccgcggtgacggcaccgaagtcgagcgggccgtcgtgccgttcgcgccggaggtcgtcgaggagctgcgggccgtggcgcgccgggccggtaccagtgcggcgaccgtgctgcacgtggcgtgggcgcgggtgctggccgcggtgtcggggcactcggacgtggtgttcggcacggtcctcttcggccggatgaacgccggtgcgggggccgaccgggtcgccgggctctacatgaacacgctcccggtgcgggtgcgcacgggtgagctgggcgccctggaggccgtgaccgcgatgcgcggtcagctggccgggctactggaacacgagcacgcctcgctggccctggcccagcaggccagtggcctgacgggcaactctcccgtgttcacctcgctgctcaactaccgccacaacaccggccgggacgacgctcccgagggcggcgacgctcccgagggcatccgtgtgctgtcctcgcgggagcgcacgagctaccccctcggtgtgtccgtggacgactacggcgactcgatgtcgctggccgtggacgccgtcgcgccgatcgacgccgtggccgtgggcacgctcctgcggaccgccgtcgagggcctggtgccgctgatcgggcaggcgctcgacggcggaccggacacggcgctggccgccgtcgacgtactggccccggaggacctgaaccggctgttggtggagtggaacgacacggtggccgaggtcgagccgtcgacgcttccggcgctgttcgcggcgcaggtggagcggacgccc

SEQ ID NO: 67 - protein, 472 aa, Streptomyces aizunensis strain NRRL B-11277
ADIYPLAPLQEGLLFHHLLAEGGVDAYVLPTVIEFDAPERLDAFTAALQQVIDRHDIYRTSIVWEGLREPVQVVWRRAELPVTEVTLAGRSDDRVAELVAHVGADMDLGRAPMITVHTAAVPGADRLLALVRLHHMVQDHTALEALLDEVQAFMTGHGSALPEPLPFRSFVAQARSGEARAEHERYFRDLLADVDEPTAPYGMADVRGDGADSVAAVVEIAPELNDRLREVARKAGTSAATVMHVAWARVLAAVSDRQDVVFGTVLLGRMSAGAGAGRVLGPYINTLPVRVRTGELGALAAVSAMRGQLAELLEHEHASLAVAQQVSGVGGDTPLFTSLFNYRHNAAPKAAEPGETPSEGLSGIRQLFTQERTNYPLSISVDDDGDSLALAVDAVAPVDPIAVGVLLRTVTEHLVDALEVSLAGGADRQLGSLPVLAEEQLRQVLAEWNDTAAEVPPVPVTALFEAWVARTP

SEQ ID NO: 68 - DNA, 1416 bp, Streptomyces aizunensis strain NRRL B-11277
gccgacatctacccgctggccccgctccaggagggtctgctcttccaccacctgctggccgagggcggcgtggacgcctacgtcctgccgacggtcatcgaattcgacgcgccggagcggctcgacgccttcaccgcggcactccagcaggtgatcgaccggcacgacatctaccgcacatcgatcgtctgggagggactgcgggagcccgtccaggtcgtctggcgccgcgccgaactgcccgtcaccgaggtgaccctcgccgggcggtccgacgaccgggtcgccgagctggtggcccacgtgggcgccgacatggacctaggccgcgccccgatgatcacggtgcacaccgccgcggtccccggggccgaccggctgctcgccctggtacgcctgcaccacatggtgcaggaccacaccgcgctcgaagccctcctcgacgaggtgcaggccttcatgaccgggcacggaagcgcgctccccgagccgctgccgttccggagcttcgtggcgcaggcccgcagcggcgaagcccgcgcggagcacgagcggtacttccgcgacctgctcgcggacgtcgacgagccgaccgccccgtacgggatggccgacgtccggggcgacggcgccgattcggtggcggccgtggtggagatcgcaccggaactgaacgaccggctgcgcgaggtcgcccgcaaggcgggcaccagcgccgccaccgtcatgcacgtggcctgggcccgggtgctcgcggcggtctcggaccgccaggacgtcgtcttcggcaccgtgctgctcggccggatgagcgcgggcgccggcgcgggccgcgtcctcggcccctacatcaacaccctcccggtgcgcgtccgcacgggcgagctgggcgccctggcagcggtctccgcgatgcggggacagctggccgaactgctggagcacgagcacgcctcgctcgccgtggcgcagcaggtcagcggagtgggcggggacacccccctgttcacctcgctcttcaactaccgccacaacgcggccccgaaggccgccgagcccggcgagacgccgtccgaaggcctctccggcatccggcagctgttcacgcaggagcgcaccaactacccgctgagcatctcggtcgacgacgacggcgattcgctcgccctcgccgtcgacgcggtcgccccggtcgacccgatcgcggtcggcgtgctgctgcgcaccgtcaccgagcacctggtcgacgcactggaggtgtcgctcgcgggtggcgcggaccggcagctcggctcgctcccggtgctcgccgaggagcagctgcgccaggtcctcgccgagtggaacgacaccgcggccgaggtccccccggtaccggtgaccgcactgttcgaggcctgggtcgcacggaccccc

SEQ ID NO: 69 - protein, 480 aa, Streptomyces aizunensis strain NRRL B-11277
ADVYPLAPLQEGLLFHHLLAGDGEDAYALPMVLEFASRDGLDAFLHALQQVVDRHDILRTGIVWDGLREPVQVVRRRAELTVEEVTLGARGTDAERVAELVAFGSTAMDLSRAPLIDLHLARATEDETDGRWLALIRAHHMVQDHTAKEVLLEEVHAFLTGHGDELAQPLPFRNFVAQARGAVDESEHERYFAELLGDVTEPTAPYGVLEARGDGSGVIRGGLTLPRPLTLRIREIARRMGVSSAPVLHVAWARVLAAVSGRTDVVFGTVLFGRMNAGAGSDRVPGPFMNTLPVRLRTAELGSLDAVSAMRGQLAELLEHEHAPLALAQRASGVHGNSPLFTSFFNYRHSQGAARGAEAEAAAAAADPVTGIRMVFEHDRTNYPLSVAVTDDGDSLDVTIDSIAPIDPHAVSALMHVTVANLVAALETALDGGADAPLDGVDVLGDAERGKVLVEWNDSAADLGQALVPELFAARAARTP

SEQ ID NO: 70 - DNA, 1440 bp, Streptomyces aizunensis strain NRRL B-11277
gccgacgtctaccccctcgccccgctccaggagggcctgctcttccaccacctgctcgcaggtgacggcgaggacgcctacgccctcccgatggtcctggagttcgcctcccgcgacggcctcgacgccttcctgcacgcactgcagcaggtcgtcgaccggcacgacatcctgcgcaccggcatcgtctgggacggcctgcgcgaacccgtccaggtggtccggcgccgcgccgaactgaccgtcgaggaagtgaccctcggcgcccggggcaccgacgccgagcgggtggccgagctggtcgccttcggcagcaccgccatggacctcagccgcgcccccctgatcgacctgcacctggcccgcgccaccgaggacgagaccgacggccgctggctcgccctgatccgcgcgcaccacatggtccaggaccacaccgcgaaggaggtcctgctggaggaggtgcacgccttcctcaccgggcacggcgacgagctggcacagccgctccccttccgcaacttcgtggcacaggcgcgcggcgcggtcgacgagtccgagcacgagcgctacttcgccgaactgctcggcgacgtcaccgaacccaccgcgccgtacggcgtactcgaagcacgcggcgacggcagcggcgtgatccgcggcgggctcaccctgccgcgccccctgaccctgcggatcagggagatcgcccggcgcatgggcgtcagctcggcccccgtcctgcacgtcgcctgggcgcgcgtgctcgcggccgtctcgggccgtacggacgtggtcttcggcaccgtcctgttcggccggatgaacgcgggcgcgggttccgaccgcgtccccggccccttcatgaacaccctccccgtccgcctgcgcaccgccgaactcggctccctggacgccgtgtccgcgatgcgcggccagctcgccgagctgctggagcacgagcacgctccgctggcactggcccagcgcgcgagtggcgtccacggcaacagcccgctgttcacgtccttcttcaactaccggcacagccagggcgccgcgcggggcgcggaggccgaggcggcggccgccgcggccgacccggtcaccgggatccggatggtcttcgaacacgaccgcaccaactacccgctgtccgtggcggtcacggacgacggcgactcgctcgacgtgaccatcgactcgatcgcccccatcgacccgcacgcggtcagcgcgctgatgcacgtgaccgtcgcgaacctcgtggcggcgctcgaaacggccctcgacggcggggccgacgccccgctggacggcgtggacgtgctcggtgacgcggagcgcggcaaggtcctggtggagtggaacgactccgccgccgacctcggccaggccctggtgccggaactgttcgcggcgcgggcggcccgtacgccc

SEQ ID NO: 71 - protein, 466 aa, Streptomyces aizunensis strain NRRL B-11277
ADVYPLAPLQEGLLFHHLLAEGEEDTYVASAVLEFDSAERVDSFAGALQQVLDRHDIFRTAIVWEGLSQPLQVVWRHAELPVTEVTLDPEAADPVAELPAVVGRTMDVRRAPLLGLHVATAADGRHLVLLRLHHLVQDHTTVEVLLSEIGAFMGGRGAELPQPLPFRDAVVQTRAGLADGGHDQYFAGLLGDVTEPTAPFGLLNTREPGMDTAETEVRIAPDLERRLRRIARKVGASPATVMHVAWARVLAAVSGRDDVVFGTVLFGRMGADADEARVLGPFMNMLPVRVKTGQLGVLEAVGAMRSQLAALMEHEHAPLAVAQNASGVEGETPLFASMFNYRHNDRSVSKSGALFDGVELRLYRELTNYPIAVNLDDDEDGMRLTVDAAAPANPRQVGTLLTTATRNLLDALERAAAGGPEVALDAVEVLDAAERRRVLVEWNDTAVEGDASSLPGLFEAQVARTP

SEQ ID NO: 72 - DNA, 1398 bp, Streptomyces aizunensis strain NRRL B-11277
gcggacgtctacccgctggccccgctgcaggagggcctgctcttccaccacctgctggccgaaggcgaagaggacacctacgtcgcctccgccgtcctggagttcgactccgccgagcgggtggactccttcgccggagccctccagcaggtgctggaccggcacgacatcttccgcaccgcgatcgtctgggaagggctgtcccagcccctccaggtcgtctggcgccacgcggagctgccggtgaccgaggtgaccctcgacccggaggcggccgacccggtggccgagctgccggcggtcgtgggccggaccatggacgtacggcgggcgccgctgctcggactgcacgtggccacggccgcggacggccggcacctggtgctcctgcgactgcaccacctggtccaggaccacacgacggtcgaggtgctgctctccgagatcggcgccttcatgggcgggcgcggcgcggaactgccccagccgctgcccttccgcgacgccgtggtgcagacccgggccggactggccgacgggggccacgaccagtacttcgccggtctcctcggtgacgtcaccgagccgaccgcgccgttcgggctgctgaacacgcgagagcccggaatggacaccgccgagaccgaggtgcggatcgccccggacctggagcgccgactgcgccggatcgcgcgcaaggtgggcgccagccccgcgaccgtcatgcacgtggcgtgggcgcgcgtgctggccgcggtcagcggccgcgacgacgtggtgttcggcaccgtgctgttcggccggatgggcgccgacgccgacgaggcccgggtgctcggccccttcatgaacatgctcccggtgcgggtgaagaccgggcagctgggcgtgctggaagcggtgggcgccatgcgcagccagctggccgcgctcatggagcacgagcacgcgccgctggccgtcgcccagaatgccagtggtgtggagggcgagaccccgctcttcgcctcgatgttcaactaccggcacaacgaccgttccgtgagcaagagcggagccctcttcgacggcgtcgaactgcggctctaccgcgagctgaccaactacccgatcgcggtgaacctcgacgacgacgaggacgggatgcgcctgaccgtcgacgcggccgccccggcgaacccgcgccaggtgggcacgctgctcaccacggcgacgaggaacctgctggacgcgctggagcgggcggcggcgggcggccccgaggtcgcgctggacgcggtcgaggtgctggacgcggccgagcgccggcgggtgctggtggagtggaacgacaccgcggtggagggcgacgcgtcctccctgcccgggttgttcgaggcgcaggtggcgcggacgccg

SEQ ID NO: 73 - protein, 468 aa, Streptomyces aizunensis strain NRRL B-11277
ADVYPLAPLQEGLLFHHILAEGGEDAYVLRAALEFDTKARLDAFVEALQLVVDRHDIFRTSFVWEGLREPVQVVWRSAVLPVNEVTLDPATTNPVADLQAIVGSTMDLGRAPLISVDYARLPDGGWMALARMHHTVRDHTALEVVLSEVRTIVTGRTAELPAPLPFRNFVAQARGGVPRVEHERYFTGLLGDVTEPTAAYGLLNVRGDGADTVHEVTEFGSELSARLRESARRIGTSPATLLHVAWARVLGVVSGRDDVVFGTVLYGRMNAGVGADRVPGPFMNTLPVRFLAGRDGVLDSVAAMRDQLAELLEHEHAPLVVAQRASGLVGDSPLFTSFINYRRNEGQSPEERSGAVMEGTRLLLSRERTNYPLVILVDDNDDGIEVAVDAVAPIDSAAVGALVRTAAESLVAALETALDGGPQALLSAVRVLEDDERTRLLTEWNDTAAEVPADTVAALFGAQVARTP

SEQ ID NO: 74 - DNA, 1404 bp, Streptomyces aizunensis strain NRRL B-11277
gccgacgtctacccgctggccccgctccaggagggcctgctcttccaccacatcctggccgagggcggcgaggacgcctacgtgctgcgggcggccctggagttcgacaccaaggcacgtctggacgccttcgtcgaggcgctccagctggtggtggaccggcacgacatcttccggacctcgttcgtctgggagggactgcgcgagccggtccaggtggtctggcgcagcgccgtcctgccggtgaacgaggtgaccctcgacccggcgaccaccaacccggtggccgatctgcaggcgatcgtcggctccaccatggacctcggccgggccccgctgatctcggtggactacgcccggctgccggacggcggctggatggccctcgcccggatgcaccacacggtccgcgaccacaccgcgctggaggtcgtgctcagcgaggtgcggacgatcgtgaccggacgcacggcggagctgcccgccccgctgccgttccgcaacttcgtcgcccaggcgcgcgggggtgtcccgcgcgtggagcacgagcggtacttcaccggtctgctcggcgacgtgacggagccgaccgcggcgtacggactgctgaacgtgcgcggcgacggagccgacaccgtccacgaagtcaccgaattcggctcggagttgagcgcccgcctgcgggagagcgcgcgccggatcgggaccagcccggccaccctgctgcacgtggcgtgggcgcgcgtcctcggcgtggtcagcggtcgcgacgacgtggtcttcggcaccgtgctgtacggccggatgaacgccggtgtcggcgccgaccgggtgcccggaccgttcatgaacaccctgccggtacggttcctggccgggcgggacggggtcctggactccgtcgccgccatgcgcgaccagctggccgagctgctcgaacacgagcacgcgccgctggtcgtcgcgcagcgggccagcggcctcgtcggcgacagcccgctgttcacctccttcatcaactaccgccgcaacgaggggcagagccccgaggagcggtcgggcgcggtgatggagggcacccggctgctgctctcgcgggagcgcaccaactacccgctcgtgatcctcgtcgacgacaacgacgacggcatcgaggtcgccgtggacgcggtggccccgatcgactccgccgcggtgggcgcgctggtccgtaccgccgccgaaagcctggtggcggcgctggagacggcgctggacggcggcccgcaggcgctgctgagcgcggtccgggtgctggaggacgatgagcggacccgtctgctcaccgagtggaacgacacggcggccgaggttccggccgacacggtcgccgccctcttcggcgcccaggtggcccgtaccccc

SEQ ID NO: 75 - protein, 469 aa, Streptomyces aizunensis strain NRRL B-11277
ADVYPLAPLQEGLLFHHMLADGGEDAYVMPAVLEFDSRERLVAFADAVGKVVNRHDIYRTSYIWQGLREPVQVVLRTATLPVTQVTLDPSGADPIAELVELGGHSMDLSSPPLITLHTAALPGSGKWLALIRAHHMVRDHTALVVLLTEVQAFVTGKEAELPEPLPYRNFVAQARGGVPQSEHARYFEALLGDIEEPTAPFGLVDVRGDGAALVRSRVASAPEVDVRLREVARRLGTSPATVLHVVWARVLSAVSHGQDVVFGTVLSGRMNAGEGADRIPGPFINTLPVRMGLDGLGVIAAVSAMRSQLAELLEHEHAPLVVAQRASKVDGGSPLFTTFLNYRRNAGQNLDERWDGELFGTRLVFARERTNFPLALMVDDNGVDIDLSIDAVAPADGVAVGELVRHAAANLVSALETALATGVETPLGAVDVLGEDERGRILGEWNDTGRPLAAATVPEMFAAQVARTP

SEQ ID NO: 76 - DNA, 1407 bp, Streptomyces aizunensis strain NRRL B-11277
gcggacgtctacccgctggccccgctgcaggagggtctgctcttccaccacatgctggccgacggcggcgaggacgcctacgtgatgcccgcggtgctcgaattcgactcccgcgagcggctggtcgccttcgccgacgccgtcggcaaggtggtgaaccggcacgacatctaccgcacctcctacatctggcagggcctgcgcgagccggtccaggtcgtgctgcgcaccgcgacgctgcccgtcacccaggtcacgctggacccgtcgggcgccgacccgatcgccgagctggtcgagctgggcggtcactcgatggacctgtccagcccgccgctgatcaccctgcacaccgcggccctgcccggcagcggcaagtggctggccctgatccgggcccaccacatggtgcgcgaccacacggccctggtggtcctgctcacggaggtccaggccttcgtgaccgggaaggaagcggagctgcccgagccgctgccgtaccgcaacttcgtcgcccaggcccgcggcggtgtgccgcagtccgagcacgcgcggtacttcgaagccctgctgggcgacatcgaggagccgacggcccccttcggcctggtggacgtgcgcggtgacggcgcggccctggtccgcagccgggtcgcctccgccccggaggtcgacgtacgcctgcgcgaggtcgcccgccggctgggcaccagcccggcgacggtcctgcacgtggtgtgggcccgggtcctctcggccgtctcccacggccaggacgtcgtcttcggcaccgtcctgtccggccgcatgaacgccggtgagggcgccgaccggatccccggccccttcatcaacaccctgccggtacggatgggcctcgacggtctcggcgtcatcgcggcggtctccgcgatgcgcagccagctggcggaactgctggagcacgagcacgcccctctggtggtcgcacagcgcgccagcaaggtggacggcggcagccccctgttcacgacgttcctcaattaccgccgcaacgcgggacagaacctggacgagcgctgggacggcgaactcttcggcacccgcctggtgttcgcccgggaacgcaccaacttcccgctggcgctgatggtcgacgacaacggcgtggacatcgacctgtccatcgacgcggtggccccggccgacggcgtcgcggtgggcgagctggtccggcacgcggcggcgaacctggtctcggcgctggaaacggcactggccacaggggtggagacaccgctcggcgcggtggacgtgctgggcgaggacgagcgcggccggatcctcggcgagtggaacgacaccggccggcccctggccgcggcgaccgtaccggaaatgttcgccgcccaggtggcgcggacaccg

SEQ ID NO: 77 - protein, 469 aa, Streptomyces aizunensis strain NRRL B-11277
ADIYPLAPLQEGLLFHHLMAEGGEDAYLMPVVVEMDSRDRVAEFARAFQLVVDRHDILRTSFVWEGLREPVQVVWREAVLPVTEVVLDPEATDPAAALQSAVGLSMDLGRAPLISVHAAALPDGERWLVLLRLHHLVQDHTALELLLHEVQVFLAGRGDELPRPLPFRDFVAQVRGGMERGEHERYFAELLGDVTEPTAPFGLIDVRGDGTEVERAVVPLASEAVERLRSVARRVGTSPATVLHVAWARVLAAVSGHSDVVFGTVLFGRMNAGAGADRVAGLYLNTLPVRVRTGELGALEAVTAMRGQLAGLLEHEHASLALAQQASGLTGNTPVFTSLINYRHNTGAETGKEGYARPEGMRMLSSRERTNYPLGVSVDDNGDSMSLAVDAIAPIDAQAVGLLLRSAVDGLLSLVERALDGGPDAALRSVHVLAPGELHRMLVEWNDTAVDVGSASAPELFAARVAQAP

SEQ ID NO: 78 - DNA, 1407 bp, Streptomyces aizunensis strain NRRL B-111277
gccgacatctacccgctggccccccttcaggagggtctgctcttccaccacctgatggcggagggcggcgaggacgcctacctgatgcccgtggtcgtggagatggactccagggaccgggtcgcggagttcgcccgtgccttccagctcgtggtggaccgccacgacatcctgcgcacttcgttcgtgtgggagggcctgcgcgagccggtccaggtggtctggcgcgaggccgtactgcccgtgaccgaggtggtcctcgaccccgaggccaccgacccggccgccgccttgcagagcgccgtgggcctgtcgatggacctgggccgggcgccgctgatcagcgtgcacgccgccgccctgccggacggcgagcgctggctggtactgctgcgcctgcaccacctggtccaggaccacaccgccctcgaactgctcctgcacgaggtgcaggtgttcctggcgggacgcggtgacgaactgccgcggccgctgccgttccgtgacttcgtcgcgcaggtgcgcggcggcatggaacgcggcgagcacgagcggtacttcgccgagctgctgggcgacgtcaccgagccgacggctcccttcgggctgatcgacgtccgcggtgacggcaccgaagtcgagcgggccgtcgtgccgctcgcctccgaggccgtcgagcggctgcggagcgtggcgcgccgggtcggtacgagtccggcgacggtactgcacgtggcgtgggcgcgggtgctggccgcggtgtcggggcactcggacgtggtcttcggcacggtcctcttcggccggatgaacgccggtgcgggcgccgaccgggtcgccgggctctacctcaacacgctcccggtgcgggtgcgcacgggtgaactcggcgccctggaggccgtgaccgcgatgcgcggtcagctggccgggctgctcgaacacgagcacgcctcgctggccctggcccagcaggccagcggcctgacgggcaacacccccgtcttcacctcactgatcaactaccgccacaacaccggtgcggagaccgggaaggagggatacgcgcgccccgagggcatgcggatgctgtcctcccgcgagcgcaccaactatccgctcggtgtgtccgtcgacgacaacggcgactcgatgtcgctggccgtggacgccatcgcgcccatcgacgcgcaggccgtcggcctgctgctgcggtcggccgtcgacggactgctctccctggtcgagcgggccctggacggcggaccggacgcggcgctgcgcagcgtccacgtcctggcccccggagaactgcaccggatgctggtggagtggaacgacacggcggtcgacgtcgggtcggcctcggcacccgaactgttcgcggcgcgggtggcgcaggccccc

SEQ ID NO: 79 - protein, 472 aa, Streptomyces aizenensis strain NRRL B-11277
ADIYPLAPLQEGLVFHHVLADGGEDAYVTPVVFALDSKQLLEDFTAALQQVVDRHDIFRTSLVWEGVREPVQVVWRRAPLSVEEVTLDADGADPVGQLLAAGGMSMDLGRAPLLTLHVAAEPGGDRWLMLLKVHHAVQDHTAMEVVLGEVQAFLTGRGGSLPQPVPFRNFVAQARGGVARSEHERYFAELLGDVEESTAPFGLVDVLGDGAGVTRVHVPFGPELHTRLRAVSQRLSTSAATVLHVVWARTLAAVSGRSDVVFGTVLFGRMNSGAGSANMPGPFINTLPVRVRTGELGALEAVGAMRGQLAELLEHEHASLAQAQRASGLAGDTPLFTALFNYRHNAGGSDQAANDERSRGFEGFTAVHSRERTNYPLAVSVDDDGESISLAVDAVAVIDGEAFGRLMRSVAENLVTVLESALDGGPEPRLSAIGVLDGTAARRMLVDWNDTALDIEPATAAELFAAQVARAP

SEQ ID NO: 80 - DNA, 1416 bp, Streptomyces aizenensis strain NRRL B-11277
gccgacatctacccgctcgcccccctccaggagggcctggtcttccaccacgtcctcgcggacggcggcgaggacgcctatgtgaccccggtggtcttcgccctcgactcgaagcagctgctggaggacttcaccgcggctctccagcaggtggtggaccggcacgacatcttccgcacctccctggtgtgggaaggcgtccgcgaaccggtccaggtggtctggcggcgggctccgctctccgtcgaggaagtgaccctggacgcggacggcgccgacccggtcgggcagctgctggcggccggcggcatgtcgatggacctgggccgggccccgctgctcaccctgcacgtcgccgcggaaccgggcggcgaccgctggctgatgctgctcaaggtgcaccacgccgtccaggaccacacggccatggaagtggtcctcggtgaggtccaggccttcctgaccgggcgcggcggctcgctgccgcagccggtgccgttccgcaacttcgtggcacaggcccgcggcggtgtggcgcgctccgagcacgagcggtacttcgcggagctgctcggtgacgtcgaggagtccaccgcgccgttcggcctggtcgacgtcctgggcgacggagcgggcgtgacccgggtgcacgtgcccttcgggccggaactgcacacccggctgcgcgcggtctcgcagcggctgagcacgagcgccgcgacggtgctgcacgtggtgtgggcgcggacgctggcggccgtgtccggccgcagcgacgtggtgttcggcacggtgctcttcggccggatgaactccggcgcgggttcggcgaacatgccgggtccgttcatcaacacgctgccggtgcgggtgcgcaccggtgagctgggcgcgctggaggccgtgggcgcgatgcgcggccagctggcggaactgctcgaacacgagcacgcctccctggcccaggcccagcgcgccagcggactggcgggagacaccccgctgttcaccgccctgttcaactaccgccacaacgccggtggttcggaccaggccgcgaacgacgagcggagccgcggattcgaagggttcacggcggtgcactcccgagagcgcaccaactaccccctcgcggtgtccgtcgacgacgacggcgagtcgatctcgctggccgtcgacgcggtggccgtcatcgacggggaggccttcggacggctgatgcgctccgtcgccgagaacctggtcaccgtactggagtcggcactcgacggcgggccggagccgcgcctcagcgcgatcggcgtcctcgacgggactgccgcgcgccggatgctcgtggactggaacgacacggcgctcgacatcgaaccggccacggccgccgagctgttcgccgcgcaggtggcccgggccccg

SEQ ID NO: 81 - protein, 473 aa, Streptomyces aizunensis strain NRRL B-11277
ADIYPLAPLQEGLLFHHMMADGGEDAYVSKEVFELDSKDRLDTFVHALQQVLDRHDIFRTALVWEGLRAPVQVVWRRAPLSVEEVTLDADGADPVGQLLAIGGTSMDLGRAPLFTAHIAPVPGGDHWLLLLKVHHAVQDHVALEVLHHEVQAFMTGEAGTLPEPVPLRNFVAQARGGVAEEEHERYFAELLGDVDEPTAPFGLVDVHGDGADTLWVRRPLEAELHTRLREVSQRLGTSAATVLHVAWARTLAAVSGRTDVVFGTVLFGRMNSGAGADRVPGPFINTLPVRVRTAELGTLASIGAMRVQLAELLEHEHASLALAQRASGLVGDTPLFTAIFNYRHNAGGSGEAAATDRNRGFEGLTLRYARERTNYPLVVAVDDNGDSIELTLDAVAAIDVHAVGRLMCTATENLVTALEQELDGGTEQPLAAVRVLGEDDLHRILDEWNDTGRDAIATVPVPELFAAQVARFP

SEQ ID NO: 82 - DNA, 1419 bp, Streptomyces aizunensis strain NRRL B-11277
gccgacatctacccgctggcccccctccaggagggcctgctcttccaccacatgatggcggacggcggcgaggacgcctacgtcagcaaggaggtcttcgaactcgactcgaaggaccggctggacaccttcgtccacgcactccagcaggtgctggaccggcacgacatcttccgcaccgccctggtgtgggagggactgcgcgctccggtccaggtggtctggcggcgggccccgctctccgtcgaggaagtgaccctggacgcggacggcgccgacccggtcgggcaactgctcgcgatcggtggcacgtcgatggacctgggccgggcgccgctgttcacggcgcacatcgccccggtgccgggcggcgaccactggctgctgctgctgaaggtgcaccacgccgtccaggaccacgtggccctggaggtcctgcaccacgaggtccaggccttcatgacgggtgaggccggcacgctcccggagccggtgcccctgcgcaacttcgtcgcacaggcccgcggcggcgtggcggaagaggagcacgagcgctacttcgccgaactgctcggcgacgtcgacgagccgaccgcgccgttcggcctggtcgacgtccacggcgacggagcggacaccctctgggtgcgccgccccctcgaagcggaactgcacacccggctgcgcgaggtctcgcagcggctgggcacgagcgccgcgaccgtgctgcacgtcgcctgggcgcggacgctggcggccgtctcgggccgcaccgacgtggtgttcggcacggtgctcttcggccggatgaactccggcgccggagccgaccgggtccccggaccgttcatcaacaccctgcccgtacgggtccgcaccgccgaactgggcacgctggcgtccatcggagcgatgcgcgtacagctggcggaactgctggaacacgaacacgcctccctggcgctggcccagcgggccagcggactggtgggggacaccccgctgttcaccgccatcttcaactaccgccacaacgccggcggttccggagaggcggcggccaccgaccgcaaccgcggcttcgaaggcctcaccctgcgctacgcccgggagcgcaccaactacccgctcgtggtggcggtcgacgacaacggcgactccatcgagctgaccctggacgcggtggcagccatcgacgtgcacgccgtgggacggctgatgtgcaccgcgaccgagaacctcgtcaccgccctggaacaggaactcgacggggggaccgaacagccgctggccgcggtgcgcgtcctcggcgaggacgacctgcaccgcatcctcgacgagtggaacgacaccggccgggacgccatcgccacggtgcccgtaccggaattgttcgcggcgcaggtcgcccgcttcccg

SEQ ID NO: 83 - protein, 476 aa, Streptomyces aizunensis strain NRRL B-11277
ADVYPLGPLQEGLLFHHLLADGGDDTYVMPAVLEFASRTGLDTFLAAFQNVLDRHDIFRTSFVWEGLPDPVQVVWRQATMPVTDVTLPEDGADPVAGLIATVGLAMDLGRAPLITVHRAAVPGDDRWLALVRIHHLVQDHMALELLLSEVGAFLAGRGADLPEPLPFRNFVAQARGESEVGGHQEYFTELLADFDEPSAPYGVTDVRGDGGAVTRAVVPFAPESERRLREVARRLRVSAATVLHVAWARTVGAMSGRDDVVFGTVLFGRMSGGAGAGHVPGPFINTLPVRVRTTDTGVLTAVSQMRTQLSRLLEHEHASLATAQQASGLESEAPLFTAFLNYRHNTARSEEQEVEEQPSEQQPVALEARLLYARERTNYPLGVAVDDDGDRLALTVDAVGTIDPEPVGVMVRATTERLVQALETALDGGPEVALSALPVLDEAGLDQVVRAWNDTAVEVEASTLPGLFGAQVLRTP

SEQ ID NO: 84 - DNA, 1428 bp, Streptomyces aizunensis strain NRRL B-11277
gcggacgtctacccgctcgggccgctccaggagggtctgctcttccaccacctgctcgcggacggcggcgacgacacctacgtgatgcccgcggtcctcgaattcgcctcgcgcaccggactcgacaccttcctcgcggcgttccagaacgtcctggaccggcacgacatcttccggacgtcgttcgtctgggaagggcttccggacccggtccaggtggtctggcggcaggcgacgatgcccgtcaccgacgtcaccctccccgaggacggcgcggacccggtcgccggtctgatcgcgaccgtgggcctggcgatggacctgggccgcgcaccgctgatcaccgtgcaccgggccgccgttcccggcgacgaccgctggctggcgctggtccgcatccaccacctggtgcaggaccacatggccctggagctgctgctctcggaggtcggcgccttcctcgccggccgcggggcggacctccccgagccgctgcccttccgcaacttcgtggcgcaggcgcgcggcgagagcgaagtcggcggacatcaggagtacttcaccgagctgctggctgacttcgacgagccgtcggccccgtacggggtcacggacgtgcgcggcgatggtggggcggtgacgcgcgcagtggtgcccttcgccccggagtccgagcgtcgactgcgcgaggtggcccgtcggctgcgcgtgagcgccgcgaccgtcctgcacgtggcctgggcccggacggtcggtgcgatgtccggccgggacgacgtggtgttcggaacggtgctgttcggccggatgagcggcggcgccggagccggtcacgtccccggcccgttcatcaacaccctcccggtgcgtgtccgtacgaccgacaccggtgtgctgacggcggtgtcgcagatgcgcacccagctgtcgcggttgctggagcacgagcacgcctcgctcgcgacggcccagcaggccagcgggctggaaagcgaagctccgctcttcaccgcgttcctcaactaccgccacaacacggcacggagcgaggaacaggaggtcgaggagcagccttcggagcagcagcccgtcgccctggaagcccgcctgctgtacgcacgcgaacggaccaactacccgctcggcgtcgcggtggacgacgacggcgatcgcctcgccttgacggtcgatgcggtgggcacgatcgacccggagccggtcggcgtcatggtccgcgcgacgacggagcgactggtccaggcgctggagacggcactggacggcggtcctgaggtggcgttgagcgcgctgccggtcctggacgaggcggggctggaccaagtcgtgcgtgcctggaatgacacggcggtggaggtcgaggcttcgacccttccgggtctgttcggcgcgcaggtgctgcggactccg

SEQ ID NO: 85 - protein, 479 aa, Streptomyces aizunensis strain NRRL B-11277
ADVYPLAPLQEGLLFHHLLADGGDDAYVQPNVLEFGSRDLLDRFAEALQRVLDRHDILRTGIVWDGLTEPVQVVWREAELPVHEVALDPQSGDLAEALAEAVGLSMDLSRAPLVELHMAAGRDGRWLGLVRVHHMVQDQTALDLLLGEIAAFLTGRGDELPEPLPFRDFVAQARGGVTREEHERYFATLLSDVTEPTAPYGLTDVSGVGAGAERAVRVLEPGLDERLRSVSRRLGASVATVMHVAWARVLGAISGRDDVVFGTVLFGRMNAGAGADRVPGPFINTLPLRVPSDRLGVLKTVSAMRDQLAQLLEHEHASLAVAQQASGITAGTPLFTSFLNYRHVLDRESVTQVEDGPVGNEPVAEEASAGIQTLLSRETSNYPLAVSVNDSGRVIRLAVEAVAHADPEAVGALLSTALEGLVGALEMALDGGPEVPLSAVPVLDEAGLHRVVEEWNDTAVEVAADTLPGLFQAQVARTP

SEQ ID NO: 86 - DNA, 1437 bp, Streptomyces aizunensis strain NRRL B-11277
gccgacgtctatccgctggcccccctccaggagggtctgctcttccaccacctcctcgccgacggcggcgacgatgcctatgttcagccgaacgtcctggagttcggctcccgcgacctgctcgaccggtttgccgaggccctccagcgggtgctggaccggcacgacatcctgcgtaccgggatcgtctgggacggcctgaccgagccggtccaggtcgtgtggcgcgaggccgaactgcccgtccacgaggtggccctcgaccctcaaagcggtgacctcgcggaagcgctggccgaggccgtcggactgtcgatggacctgtcccgggcgccgctggtcgagctgcacatggccgccggccgtgacggccgctggctcggcctggtgcgggtccaccacatggtgcaggaccagacggcactggacctcctgttgggcgagatcgcggcattcctcaccggcagaggtgacgagctgcctgaaccgctgccgttccgggacttcgtggcacaggcccgcggcggagtgacgcgggaggagcacgagcgctacttcgcgacgttgctcagtgacgtgacggagcccacggccccttacggtctgaccgatgtgagcggtgtcggggctggtgccgaacgcgcggtcagggtgctggagcccggcctggacgagcggctgcgttcggtctcgcgacgcctgggtgccagtgtggccaccgtcatgcacgtggcctgggcccgtgtgctcggagcgatcagtggccgggacgacgtggtgttcgggacggttctgttcggccggatgaatgccggagcgggtgcagaccgggtacccggtcccttcatcaacaccctcccgctgcgggtgccgtcggaccgactgggtgtcctcaagacggtgtccgcgatgcgcgaccagctggcgcagctcctcgaacacgagcacgcgtcgctcgccgtggcccagcaggccagtggcatcacggcaggcactccgctgttcacctccttcctcaactaccggcacgtcctggatcgggagtccgtcactcaggttgaggacgggccggtcggcaacgaacccgtcgccgaggaggcctcagccggcatccagacgctgctctcccgtgagacgagcaactacccgctcgcggtgtccgtgaacgacagcggacgggtgatccggttggccgtggaagcggtggcccacgcggacccggaggcggttggcgcccttctgagcacggccttggaaggtttggtcggggcgttggagatggcgttggacggcgggcccgaggtaccgctgagcgcggtgcccgtgctggacgaggccggactgcaccgggtggtggaggaatggaacgacacggcggtggaggtcgccgccgacacgctgccggggctgttccaggcgcaggtcgcccgaactccg

SEQ ID NO: 87 - protein, 482 aa, Streptomyces aizunensis strain NRRL-11277
ADVYPLAPLQEGLLFHHLLADGGDDAYVLPSVLEFSSREQLGAFTDALQCVLDRHDIFRTSFVWEGLVEPVQVVWRKAVLPVVEVTLDAQSTDPVAELVDVVGTTMDLGRAPLFTVHAAALADGERWLGLVRVHHLVQDHTALEILLAEVGAFLTGKGAELPEPMSFRTFVAHARGGNENAGHEEYFARLLGDVTEPTAPYGLVDIHGAGAEVVREVVPFAPAAETRLREVSRRLGVSAATVLHVAWARALAAVSGRDDVVFGTILFGRMNAGVGADRVPGPLINTLPVRVRTDELGVLGAVSAMRDQLAQLLEHEHAPLSVAQGASGVVGDAPLFTALFNYRHNTGGSTEEETGEALTEATEGANGEGDGIRLVFSRERTNYPLAVAVDDNGHDRGLSLAVDALAPIDGAAVGVLVLTAAENLVAALETALDGGPDVSLSAVDVLDAAERERVLVEWNDTAVEVEASTLPGLFGAQVLRTP

SEQ ID NO: 88 - DNA, 1446 bp, Streptomyces aizunensis strain NRRL B-11277
gccgacgtctacccgctggcgcccctccaggagggtctgctcttccaccacctcctcgccgacggcggcgacgacgcgtacgtgctcccgtcggtcctcgaattctcctcgcgcgagcagctgggcgccttcacggacgcactccagtgcgtgctcgaccggcacgacatcttccggacctcgttcgtctgggagggactggtcgagccggtccaggtggtctggcgcaaggccgtcctgccggtcgtcgaggtgaccctcgacgcgcagagcaccgacccggtggccgaactggtcgacgtggtcggtaccacgatggacctgggccgggctccgctgttcaccgtgcacgcggccgccctggccgacggcgagcgctggctgggcctggtgcgcgtgcaccacctggtccaggaccacaccgcgctggagatcctgctcgccgaggtcggcgcgttcctcaccgggaagggcgccgaactgccggagccgatgtccttccggaccttcgtggcccacgcccgcggcggcaacgagaacgccgggcacgaggagtacttcgcccggctgctcggtgacgtgaccgaaccgaccgccccctacggcctggtggacatccacggagccggcgcggaggtcgtgcgcgaggtcgtcccgttcgctccggcggccgagacacggctgcgggaggtctcccggcggctcggcgtgagtgccgcgaccgttctgcacgtggcctgggcccgggcgctggccgcggtcagcggccgcgacgacgtcgtcttcggcacgatcctgttcggccggatgaacgccggggtcggcgccgaccgggtccccggtccgctgatcaacacgcttcccgtgcgggtgcgcaccgatgaactcggtgtgctgggcgccgtgtcggcgatgcgcgaccagctcgcgcagctcctggagcacgagcacgcgccgctctccgtggcgcagggcgccagcggggtcgtcggtgacgcgccgctgttcaccgcgctgttcaactaccggcacaacaccggtggcagcaccgaggaggagaccggcgaggctctcacggaggccaccgagggcgcgaacggtgagggcgacggcatccgcctggtgttctcccgcgagcggacgaactacccgctcgccgtcgcggtcgatgacaacggtcacgaccgtgggctgtcgctggccgtggacgccctcgcgcccatcgacggcgcggccgtcggtgtcctggtgctcaccgccgcggagaacctggtcgcggcgctggaaacggcgctggacggcggtcccgatgtgtcgctgagtgcggtggacgtgctcgacgcggccgagcgtgagcgtgtgctggtggagtggaacgacacggcggtggaggtcgaggcttcgaccctgcccggtctgttcggcgcgcaggtgctgcggactccg

SEQ ID NO: 89 - protein, 477 aa, Streptomyces aizunensis strain NRRL B-11277
ADVYPLAPLQEGLLFHHLLADGGDDDAYVMPFVMEFSSRERLDAFLDGLQKVIARHDILRTGIVWDGLTEPVQVVWREAELPVHEVALDPDNSDLADQLLTGVGLSMNLDRAPLISTHVAPIADDERWVALVRMHHIVQDHTALEVVLGELDAFLTGRGEELPEPLPFRDFVAQARSGANSGAHEKYFTELLGDVTEGTAPFGALDVRSGGAGVVRARVPFAPELEARLREVSRRLGASAATVMHVAWARALGTMSGRDDVVFGTVLFGRMNAGAASERVPGPFMNTLPVRVRTADLGVLDAVSALREQLAELLEHEHAPLAQAIQASGMAGNSPLFSALFNYRHNTELEPAPEPAPQADPEEERVGGIRMIFARELTNYPLAVSVDDDGDAISLAVDAVSPIDPQAVGALVRTAAENLVSALETALDGGPQAPLTSVEVLDAAELRRVLVEWNDTAVEVEASTLPGLFGAQVLRTP

SEQ ID NO: 90 - DNA, 1431 bp, Streptomyces aizunensis strain NRRL B-11277
gccgacgtctacccgctggcccccctccaggaaggtctgctcttccaccacctgctcgccgacggcggcgacgacgacgcgtacgtgatgcccttcgtcatggagttcagctcccgcgagcgtctcgacgcgttcctcgacgggctgcagaaggtgatcgcccgtcacgacatcctgcgtaccgggatcgtctgggacggcctgaccgagccggtccaggtcgtgtggcgcgaggccgaactgcccgtgcacgaggtggccctcgacccggacaacagcgatctggctgatcagctcctgaccggagtcggcctgtcgatgaacctcgaccgggcgccgctcatcagcacccacgtcgcgcccatcgcggacgacgagcggtgggtcgcgctggtgcggatgcaccacatcgttcaggaccacaccgcgctcgaagtggtcctcggtgagctggacgcgttcctcaccgggcggggcgaggagctgccggagccgctgccgttccgcgacttcgtcgcccaggcacggagcggggccaacagcggcgcgcacgagaagtacttcacggagctgctcggggacgtcaccgaagggaccgcaccgttcggcgcgctggacgtcaggagcggcggtgcgggcgtcgtacgagcccgggtgccgttcgctcccgagctggaggcacggctgcgggaggtctcccgtcggctcggcgccagcgcggcgacggtcatgcacgtcgcgtgggcacgtgccctcgggacgatgtccggccgcgacgacgtggtgttcggaacggtgctgttcggccggatgaacgccggtgcggcctcggagcgggtgcccggtccgttcatgaacaccctgccggtccgggtgcggaccgctgacctgggtgtgctggacgcggtgtccgcgctgcgcgagcagctggcggagctgttggaacacgagcacgcgccgctggcgcaggccatccaggccagtggcatggcggggaactcgccgctgttctcggcgctgttcaactaccgtcacaacaccgagctcgaaccggcacccgagcccgcgccgcaggccgatcccgaggaagagcgggtgggcggcatccggatgatcttcgcccgggagctgaccaactacccgctcgccgtctcggtcgacgacgacggcgacgcgatcagcctggcggtcgacgcggtctccccgatcgacccgcaggccgtcggcgccctggtgcgcacggccgcggagaacctcgtgtcggcgctggagacggcgctggacggcggtccgcaggcgccgctgacctcggtcgaggtgctggacgcggcggagctccgccgggtgctggtggagtggaatgacacggcggtggaggtcgaggcttcgacccttccgggtctgttcggcgcgcaggtgctgcggactccg

SEQ ID NO: 91 - protein, 467 aa, Streptomyces griseofuscus strain NRRL B-5429
ADVYPLAPLQEGILFHHLMSAPSGEDAYVLPMALGFDSRSRLDEFAAVLQKVVDRHDILRTAVMWEGLREPVQVVCRHAEVPVTEASLDPVPDGDVQGVVDGLLSVCGSLMDVTVAPLVHVTVAAVPGTEQWVALVQVHHLIQDHTAVDVLFAEVQAFLEGREGELAAPLPFRNFVAQARLGIPAAEHEAFFGGLLGDVMEPTAPFGITDVRGDGTAVAESRAAVSEATAAAVREVARRLGVSAATVLHVMFARVVAAVAGREDVVFGTVLFGRMQAGAGADRIPGLFINTLPVRLDTGRGGVLDAVRSMQGDLAELLVHEHAPLALAQRMSGVAAEAPLFTALFNYRHSAGATDAGMEVEGIEVLFAQERTNYPLTVSVDDTGDGFVFTVQCVAPIDPELVLSLMDTATGRLVQALDEAPETPLHTLPVLDDTHLSEVLGDWKDAGREVPVGVLPVLFEEQVARTP

SEQ ID NO: 92 - DNA, 1401 bp, Streptomyces griseofuscus strain NRRL B-5429
gcggacgtctatccgctggcgccgctccaggagggcatcctcttccaccacctgatgagcgcgccctcgggcgaggacgcctatgtccttccgatggccttgggcttcgattcccgctctcggctggacgagttcgccgcggttctccagaaggtggtggaccggcacgacatcctgcgtacagccgtgatgtgggagggcctgcgggagcccgtccaggtcgtctgccgacacgccgaagtccccgttaccgaagcttcgttggacccggtcccggacggtgacgtacagggcgtggtggacggactgctgtccgtctgcgggtcgttgatggatgtgaccgtggcgccgttggtgcatgtcacggtcgccgcggtgcccggcaccgagcagtgggtggcgctggtgcaggtgcatcacctcatccaggaccacacggcggtggacgttctcttcgccgaggtccaggccttcctggagggccgtgagggggagctggcggcgccgctgccgttccggaacttcgtcgcgcaggcgaggctcgggatcccggcggccgagcacgaggcattcttcggcggtctgctcggcgatgtcatggagcccaccgcgccgttcgggatcacggatgtgcgtggggacggcacggcggtcgccgagtcccgtgccgccgtcagcgaggcgacggccgcagcggtgcgtgaggtggctcgtcgtctgggtgtgagtgcggcgacggtgctgcatgtgatgttcgcccgggtggtcgcggcggtcgcgggccgtgaggatgtcgtcttcggcactgtgctgttcgggcggatgcaggccggcgcgggagcggaccgcatccccgggctgttcatcaacacgctgccggtccgactcgacaccggtcgtggtggggtgctcgacgcggtccgttcgatgcagggtgacctggccgaactcctggtccatgagcacgcgccgctggccctcgcccagcgcatgagtggcgtcgcggcggaggcgccgctcttcacggcgctgttcaactaccggcacagcgccggtgcgacggacgcgggcatggaagtggaggggatcgaggtcctgttcgcgcaggagcggaccaactatccgctgacggtgtccgttgacgacaccggcgacgggttcgtcttcaccgtccagtgcgtggccccgatcgaccccgagctcgtcctgtcgttgatggacaccgcgaccggccggctggtccaggccctggacgaggctccggaaaccccgctgcacaccctccccgtcctcgacgacacccacctgagtgaagtcctcggcgattggaaggacgctggccgggaggttccggtcggggtgctgccggtgttgttcgaggagcaggtggcgcggacgccg

SEQ ID NO: 93 - protein, 467 aa, Streptomyces griseofuscus strain NRRL B-5429
ADVYPLAPLQEGILFHHLMGASSGEDAYVLPMALGFDSRSRLDEFVAVLQKVVDRHDILRTAVLWEGLREPVQVVSRHAEIPVREAALEHIAEGDVQGVVDGLLAACGTLMDITLAPLVHVTVAPVPGTTRCVALVQVHHLIQDHTAVDVLFAEVQAFLEGREGELAAPLPFRNFVAQARLGIPVAEHEKFFTTLLGDVTEPTAPFAVLDVRGDGTGVAESSVALDQATAASVREVARRLGVSAATVLHVMFARVVAAVAGREDVVFGTVLFGRMQAGAGADRIPGLFINTLPVRLDTGRGGVLDAVRSMQGDLAELLVHEHAPLALAQRMSGVAAEAPLFTALFNYRHSVGAADAGMEVEGIEVLFAQERTNYPLTVSVDDTGDGFVFTVQCVDPIDPGLVLSLMDTATGRLVQALDEAPETPLHTLPVLDQARLDRIVGDWNDTGRDVSDGSLPALFEERVAQAP

SEQ ID NO: 94 - DNA, 1401 bp, Streptomyces griseofuscus strain NRRL B-5429
gccgatgtgtacccgctggcgccgctccaggagggcatcctcttccaccacctgatgggcgcctcctcgggcgaggacgcctacgtccttccgatggcgttgggcttcgattcccgctctcggctggacgagttcgtcgccgtcctccagaaggtggtcgatcggcacgacatcctgcgcaccgctgtgctctgggagggcctgcgggagccggtccaggtggtcagtaggcacgccgaaatcccggtgcgcgaggccgcgttggagcacatcgccgagggtgatgtgcagggcgtggtggacgggctgctcgcggcctgcgggaccctcatggacatcaccttggccccgctcgtgcatgtcaccgtggctccggttcccggcaccacccgatgcgtggccctcgtacaggtgcatcacctcatccaggaccacacggcggtggacgttctcttcgccgaggtccaggccttcctggagggccgtgagggggagctggcggcgccgctgccgttccggaacttcgtcgcacaggcgaggctcgggatcccggtcgccgaacacgagaagttcttcaccaccctcctcggcgatgtcacggagccgaccgcgccgttcgcggtgctggatgtgcggggcgatggcacaggcgtggccgagtcctccgtggctctcgaccaggcgacagccgcctccgtgcgtgaggtggctcgtcgtctgggtgtgagtgcggcgacggtgctgcatgtgatgttcgcccgggtggtcgcggcggtcgcgggccgtgaggatgtcgtcttcggcactgtgctgttcgggcggatgcaggccggcgcgggtgcggaccgtatccctgggctgttcatcaacacgttgccggtccgactcgacaccggtcgtggtggggtgcttgacgcggtgcgttcgatgcagggtgacctggccgagctgctggtccatgagcacgcgccgctggccctcgctcagcgtatgagtggtgtcgcggcggaggcgccgctcttcacggcgctgttcaactaccggcacagcgtgggcgcggcggacgccggtatggaagtggaggggatcgaggtcctgttcgcgcaggagcggaccaactacccgctgacggtgtcggttgacgacaccggcgacgggttcgtcttcaccgtgcagtgcgtcgatccgatcgaccccggcctcgtcctgtcgttgatggacaccgcgaccggccgactggtccaggccctggacgaggccccggagaccccgctgcacaccctgcccgttctcgaccaggcccgcctggaccggatcgtcggcgattggaacgacaccggtcgggacgtctccgacggttcgctgccggcgctgttcgaggagcgggtcgcgcaggcgccg

SEQ ID NO: 95 - protein, 467 aa, Streptomyces griseofuscus strain NRRL B-5429
ADVYPLAPLQEGILFHHLMSASSGEDAYVLPMALGFDSRSRLDEFVAVLQKVVDRHDILRTAVLWEGLREPVQVVYRRAEIPVREAALKQIADGDVQGVVDGLLATCGSLMDVTVAPLVHLTVASVPGTSRWVALVQVHHLIQDHTAVDVLFAEVQAFLEGREGELAAPLPFRNFVAQARLGIPVAEHEEFFGRLLGDVAEPTAPFGIVDVRGDGTAVAESRAEVGDETAQRLREVARGLGVSAATVLHVMFARVVAAVAGREDVVFGTVLFGRMQAGAGADRIPGLFINTLPVRLDTGRAGVLDAVNSMQGDLAELLVHEHAPLALAQRMSGVSAEAPLFTALFNYRHSAGETDAGMEVEGIEILFAQERTNYPLAVSVDDTGDGFAFTVQCVDPIDPDLVLSLMNTATRRLVQALDEAPGTALHTLPILDQSHLDLVLASWNDTRREIPGTLLPALFEEQVARTP

SEQ ID NO: 96 - DNA, 1401 bp, Streptomyces griseofuscus strain NRRL B-5429
gcggacgtgtacccgctggcgccgctccaggagggcatcctcttccaccacctgatgagcgcctcctcgggcgaggacgcctacgtccttccgatggcgttgggcttcgattcccgctctcggctggacgagttcgtcgccgtcctccagaaggtggtggaccggcacgacatcctgcgcaccgctgtgctctgggagggcctgcgggagccggtccaggtcgtgtaccggcgcgccgaaatccccgtccgtgaagccgcgttgaagcagatcgcggacggcgatgtgcagggcgtggtggacgggctgctcgcgacgtgcgggtcgctgatggatgtcaccgtggcaccgctggtgcatctcaccgtcgcgtcggtgcccggcacgagccggtgggtggcgctggtgcaggtgcatcacctcatccaggaccacacggcggtggacgtcctcttcgccgaggtccaggccttcctggagggccgtgagggggagctggcggcgccgctgccgttccggaacttcgtcgcacaggcgaggctcgggatcccggtggccgaacacgaggagttcttcggccggctgctgggtgacgtggccgagccgacagcgccgttcgggatcgtggatgtgcgcggggacggtaccgcagtcgcggagtcccgtgcggaggtcggcgacgagacggcccagcggctgcgcgaggtggcgcggggtctgggcgtgagcgcggcgacggtcctgcatgtgatgttcgcccgggtggtcgcggccgtcgcgggccgcgaggacgtggtgttcggcaccgtgctgttcgggcggatgcaggccggcgcgggagcggaccgcatccccgggctgttcatcaacaccctgcccgtccgcctcgacaccggccgcgccggcgtgctcgacgcggtcaactcgatgcagggcgacctggccgagctgctggtccatgagcacgccccacttgccctggcgcagcgaatgagcggggtctcggccgaggcaccgctgttcaccgcgctgttcaactaccggcacagcgccggtgagacggacgcgggcatggaggtcgaggggatcgagatcctcttcgcccaggaacggaccaactatccgctggcggtctcggtggacgacaccggcgacgggttcgccttcaccgtccagtgcgtcgacccgatcgaccccgatctcgtcctgtccctcatgaacaccgccacgcgtcggctggtccaggccctggacgaagcccccggcaccgccctccacaccctgccgatcctcgaccagagccacctggacctggtgctcgccagctggaacgacacacgccgggagatcccgggcacgctcctgccagcgctgttcgaggagcaggtggcgcggactccg

SEQ ID NO: 97 - protein, 467 aa, Streptomyces griseofuscus strain NRRL B-5429
ADVYPLAPLQEGILFHHLMSASSGEDAYVLPMALGFDSRERLDEFAGVLQKVVDRHDILRTAVLWEGLREPVQVVCRHAEILFREVELGQIPDGDVQGVADGLLAVRGSLMDITTAPLVHVTVAEVPGTTRWVALVQVHHLIQDHTAVDVLFAEVQAFLTGRAAELPTPLPFRNFVAQARLGIPVAEHEAFFTDLLGDVTEPTAPFGIVDVRGDGTAVAESRAAVSEATAAAVREAARRLGVSAATVLHVMFAWVVAAVAGREDVVFGTVLFGRMQAGAGADRIPGLFINTLPVRLDTGRGGVLDAVRSMQGDLAELLVHEHAPLALAQRMSGVAAEAPLFTALFNYRHSAGATDAGMEVEGIEVLFAQERTNYPLTVSVDDTGDGFVFTVQCVDPIDPDLVLSLMDTATGRLVQALDDAPGTPLHTLPVLDDTHLNQVLTRWNDTTRDTPNSALPALFEAQVARTP

SEQ ID NO: 98 - DNA, 1401 bp, Streptomyces griseofuscus strain NRRL B-5429
gcggacgtctaccccctggcgccgctccaggagggcatcctcttccaccacctgatgagcgcctcctcgggcgaggacgcctacgtactgccgatggccctgggctttgactcccgggagcggctggacgagttcgccggggttctccagaaggtggtggaccggcacgacatcctgcgcaccgccgtgctgtgggaggggttgcgggagcccgtccaggtcgtctgccggcacgccgaaatactcttccgagaagtcgagttggggcagatccccgatggggacgtacagggtgtggcggacggtctgctggcggtccgcggatcgctgatggacatcaccacggccccgttggtccatgtcaccgtggccgaggtgcccggcacgacccgctgggtggccctcgtacaggtgcatcacctcatccaggaccacaccgcggtggacgtcctcttcgcggaggtccaggcgttcctcacaggacgcgcggccgagctcccgacgccgctgccgttccggaacttcgtcgcacaggcgaggctcgggatcccggtcgccgagcacgaggcgttcttcaccgacctcctcggcgatgtcacggaacccaccgccccgttcggcatcgtggatgtgcgcggggacggcacggcggtcgccgagtcccgtgccgccgtcagcgaggcgacggccgcagcggtgcgtgaggcggctcgtcgtctgggggtgagtgcggcgacggtcctgcatgtgatgttcgcctgggtggtcgcggcggtcgcgggccgtgaggatgtcgtcttcggcaccgtgctgttcgggcgtatgcaggccggcgcgggagcggaccgtatccccgggctgttcatcaacacgctgccggtccgactcgacaccggtcgtggtggggtgctcgacgcggtgcgttccatgcagggtgacctggccgaactcctggtccatgagcacgccccgctcgccctcgcccagcgcatgagtggcgtcgcggcggaagcgccgctcttcacggcgctgttcaactaccggcacagcgcgggcgcgacggacgcgggcatggaagtggaggggatcgaggtcctgttcgcgcaggagcggaccaactacccgctgacggtgtccgtcgacgacaccggcgacgggttcgtcttcacagtccagtgcgtcgacccgatcgaccccgacctcgtcctgtcgttgatggacaccgcgaccggccgactggtccaggccctggacgatgcccccgggacgcccctgcacaccctccccgtcctcgacgacactcacctgaaccaggtcctcacccgctggaacgacaccacccgggacaccccgaactcggcccttccggcactgttcgaggcacaggtcgcgcggacgccg

SEQ ID NO: 99 - protein, 462 aa, Kitasatosporia sp strain ECO-03
ADIYPLAPLQEGIFFHHLMNAGGDDVYVLPTVLTFDSRDRLDAFLGALQQVIDRHDVLRTAVLWEGLSEPVQVVARHAVLPVELVELDGSGDDPVEQLRAACRPSMDLTSAPLLRGHIAAEPGGERWLLLVQQHHLVIDHTALEVLLTEIRAVLDGEQDRLPAPKPYRDFVAQARLGVSGEEHERFFAGLLREVTEPTGPFGQLDVRGDADGVAEARLAVAAGLGGRVREQARRLGVSAAAVFHVAWARVVAATSNRDDVVFGTVLFGRMSSGDRVPGLFINTLPVRVETAGVGVEEALLAMRGQLADLLVHEHAPLALAQKASGIGGSAPLFTALLNYRHGQGAGLEEAPETGLLGVEVTHVYERTNYPLTMSVDDFDNGFALTVQAVAPIDAELVCGLVHTALEGLVTALETAPEAALEQVEVLGDVERRRVLGEWNDTAREVPFGTLPGLFEAQVARTP

SEQ ID NO: 100 - DNA, 1386 bp, Kitasatosporia sp strain ECO-03
gccgacatctacccgctggcgccgctgcaggaggggatcttcttccaccacctgatgaacgccggcggcgatgacgtctacgtgctgccgaccgtgctgaccttcgactcacgcgaccgcctggacgccttcctgggcgcgctgcagcaggtgatcgaccggcacgacgtcctgcgcaccgctgtgctgtgggaggggttgtccgagcccgtccaggtcgtcgcgcgtcacgctgtgctgcccgtcgagctggtcgagctggacggctcgggagacgacccggtcgagcagcttcgggccgcgtgccgtccgtcgatggacctgaccagcgcgccgctgctgcgcggccacatcgccgccgagccgggtggcgagcggtggctgctgctggtgcagcagcaccacctggtcatcgaccacacggcgctggaggtgttgctgacggagatccgggccgtgctggacggtgagcaggaccggctgccggcgccgaagccgtaccgcgacttcgttgcccaggcccggctgggtgtgtcgggtgaggaacacgagcgcttcttcgcggggctcctgcgcgaggtgaccgagccgaccgggcccttcgggcagctggatgtgcgtggggacgccgacggggtggcggaggccaggctcgcggtggccgcagggctcggcggacgggtgcgggaacaggcgcgccgactcggcgtgagtgcggccgcagtgttccacgtggcgtgggcgcgggtggtcgcggcgacctcgaaccgcgacgacgtggtgttcggcaccgtgctgttcgggcggatgagcagtggggaccgggtacccggcctcttcatcaacacgctgccggtacgggtggagaccgccggggtcggggtggaggaggcgctgctcgcgatgcgcgggcagctcgcggacctgctggtgcacgagcacgccccgctcgcgctcgcgcagaaggccagcgggatcggcggctcggcgccgctcttcaccgcgctgctcaactaccgccacggtcagggagccggcctggaggaggcgcccgagaccgggctgctgggtgtcgaggtcacgcatgtgtacgagcggaccaactacccgctgaccatgtcggtcgacgacttcgacaacggcttcgcgctcaccgtccaggcggtggcgccgatcgacgccgagctggtctgcggtctggtgcacacggcgcttgagggcctggtcaccgcgctggaaaccgcacctgaggccgcccttgagcaggttgaggtgttgggggatgttgagcggcgtcgggtgttgggggagtggaacgacactgcccgtgaggttccgttcggcacgttgcctgggctgttcgaggcgcaggtggcgcgtactccg

SEQ ID NO: 101 - protein, 463 aa, Kitasatosporia sp strain ECO-03
ADIYPLAPLQEGIFFHHLMATGEGGDDIYVMPTTLTFDSRDRLDTFLDALQRVVDRHDILRTAVFWQDLSEPVQVVARQAALPVEQHELTGTDAAAELSAACPASMDLTRAPLLRAHIAAEPGSARWLLVLQRHHLVTDHTALDILLAEIQAILADEEHRLPAPLPFRDFVAQARLGVSREEHEKFFAELLGDLSEPTAPFGLLDVRGDGSRVTEAELPLGAGLAERLREQARVLGVSPATILHVAWARVVAATSNRDDVVFGTLLFGRMNAGSGADRVPGLFINTLPVRVPTAGISAVDAVLAMRGQLADLLVHEHAPLALAQQASGISAPAPLFTALLNYRHSHAAADGAEAGLAGVEAEHGQDRTNYPLTMAVDDLGTGFLLSVQAVTPIDPELVCALVHTALHGLVTALESAPDTLVEQIDVLTEAQRHTLLNEWNDTAVDIPAAGLTELFEAQVARTP

SEQ ID NO: 102 - DNA, 1389 bp, Kitasatosporia sp strain ECO-03
gccgacatctacccgctcgccccgctccaggagggcatcttcttccaccacctgatggcgaccggcgagggcggcgacgacatctacgtcatgccgaccacgctgaccttcgactcccgcgaccgcctggacaccttcctggacgcgctgcagcgcgtcgtggaccgccacgacatcctgcgcacggccgtgttctggcaggatctgtccgagcccgtccaggtggtggcccggcaggccgcccttccggtggagcagcacgagctcaccggcacggacgccgcggccgagctctccgccgcctgccccgcctccatggacctgacccgggcgcccctgctgcgggcccacatcgcggccgagccgggcagcgcgcgctggctgctggtgctccagcgccaccacctggtcaccgaccacaccgcgctggacatcctgctggccgagatccaggcgatcctggccgacgaggagcaccggctgccggcgcccctgccgttccgggacttcgtcgcccaggcccgcctgggggtctcgcgcgaggagcacgagaagttcttcgccgagctgctgggcgatctgagcgagccgaccgcgccgttcggcctgctggacgtccgcggcgacggcagccgggtcaccgaggccgagctcccgctcggcgccgggctcgccgagcggctgcgcgagcaggcccgcgtgctgggcgtgagcccggccacgatcctgcacgtcgcctgggcccgcgtcgtcgccgccacctccaaccgcgacgacgtggtcttcggcaccctgctcttcggccggatgaacgcgggcagcggcgccgaccgggtgccgggcctgttcatcaacaccctcccggtgcgcgtacccaccgcaggcatcagcgcggtcgacgccgtgctcgccatgcgcggccaactcgccgacctgctggtccacgaacacgcccccctcgccctcgcccagcaggccagcggtatcagcgccccggccccgctcttcaccgcactgctcaactaccgccacagccacgccgccgcggacggcgcggaggccggcctggcaggggtcgaggccgagcacgggcaggaccgcaccaactacccgctgaccatggcggtcgacgacctcggcaccgggttcctgctctccgtgcaggcggtcaccccgatcgaccccgagctggtctgcgccctggtgcacaccgcgctccacggcctggtgaccgcgctggagagtgcccccgacaccctggtggagcagatcgacgtcctcaccgaggcccagcgccacaccctgctgaacgagtggaacgacaccgccgtcgacatccccgccgccggcctgaccgagctgttcgaggcgcaggtcgcgcggacgccg

SEQ ID NO: 103 - protein, 464 aa, Streptomyces sp strain ECO-38
ADIYPLAPLQEGIFFHHLLDAGEGDVYIQPLVLRFDSRERLNVFTGALQCVVDRHDILRTAFVWQDLPQPVQVVLRTAPIPVQHVHWEPQPSVSFADQLVAACPASMDIGQAPLVRVFTAQEPDTGAWHALIQVHHLIQDHTTLDILLAEIQAMVTGREETLPEPLPFRDFVAQARLGTPREEHERFFAELLGDVTEPTAPFGVVDVQGDGRAVRETRLPVDAGLAGRLRMQARALGVSPATVFHLAWARLAGALSGRDDVVFGTVLFGRMNAGAGADRVPGLFINTLPVHARLGEDTVGDALQTMHRHLADLLAHEHAPLALAQQASGLPPQTPLFTSLLNYRYSGAGSDASESPLEGIELLHMHERTNFPLGVYIDDSMVGFRLTVQAADPIDADAVARWMHTVTENIVQALESAPHTPVRRVEVLSDAARHRVLVEWNETARPVAGATLVELFEARVAGAP

SEQ ID NO: 104 - DNA, 1392 bp, Streptomyces sp. strain ECO-38
gcggacatctacccgctggcgcctctccaggaaggcatcttcttccaccacctcctggatgcgggagagggcgatgtctacatccagcccctcgttctgcggttcgactcccgggagcgcctcaatgtcttcaccggcgctttgcagtgtgtggtggaccggcacgacatcctgcgcacggcttttgtgtggcaggacctgccccagccggtgcaggtcgtcctgcgcacggcaccgatcccggtccagcacgtccactgggagccgcagccgtcggtgagcttcgcggaccagttggtggcggcctgcccggcgagcatggacatcggacaggcgccccttgtgcgggtgttcaccgcgcaggagccggacacgggtgcgtggcatgccctgatccaggtgcatcacctgatacaggaccacaccaccctggacatcctgctggccgagatccaggcgatggtgacggggcgggaggagacccttccggagccgcttccgttccgtgacttcgtcgcgcaggcccggctcggtacccctcgcgaggagcacgagcggttcttcgccgagctgttgggagacgtcacggagccgaccgcgccgttcggtgtggtggatgtgcagggtgacggcagggcggtccgggagaccaggctgcccgtggatgcgggcctcgcggggcggctgcggatgcaggcccgtgcgctcggcgtcagcccggccaccgtcttccacctggcctgggctcgtctggccggagcgctgtccgggcgtgacgacgtggtcttcggtacggtcctgttcgggcggatgaacgctggtgccggcgccgaccgcgtccccggcctgttcatcaacacccttcccgtgcacgcgcgtctgggcgaagacaccgtcggcgacgcgctgcagaccatgcaccgtcacctggcggacctcctcgcccatgagcacgcgccgctcgcgctcgcgcagcaggccagtggactgccgccgcagacaccgctgttcacctcgctgctgaactaccggtattccggggcgggttccgacgcgtccgagagcccgcttgagggcatcgagctgctgcacatgcacgagcggaccaacttcccgctgggtgtgtacatcgacgactcgatggtcggtttccggctgaccgttcaggccgccgaccccatcgatgccgatgctgtcgcccggtggatgcacacggtcacggagaacatcgtccaggccctggagtccgctccgcacacaccggtgcggcgggtggaggtgctgagcgatgccgcccgtcaccgtgtgctggtggagtggaatgagacggcccggccggtggcgggtgcgacgttggtggagttgttcgaggcgcgggtggcgggtgcgccg

SEQ ID NO: 105 - protein, 470 aa, Streptomyces sp. strain ECO-38
ADIYPLAPLQEGIFFHHLLDAGEGDVYIQPLVLRFDSRERLDVFTGALQRVVDRHDILRTAFVWQDLPQPVQVVLRTASLPVETVVLGTAGNPAEQLVAACPASMDIGQAPLIRVFIAQEPDTGAWHALIQTHHLILDHTTMDVLMGEIQAVVAGREESLPEPLPFRDFVAQARLGTPREEHERFFAELLGDVTEPTAPYDLVDVQGDGADVAERKLSLDAGLATRLRSRARALGVSPATVFHLAWARLIGTLTGRDDVVFGTVLFGRMNAGAGADRVPGLFINTLPVRTRLHEDTVGDALQTMHRHLADLLAHEHAPLALAQQASGLPPQTPMFTSLLNYRHSAEAATGSSNAPEGVELLYARERTNYPLGVYIDDTTVGFRLTVQAADPIDADAVARWMRTVTENIVQALESAPDTPMRRVEALDDADRHRLLVEWSGGGAEPVSASASEPASATLVELFEAQAAGAP

SEQ ID NO: 106 - DNA, 1410 bp, Streptomyces sp. strain ECO-38
gcggacatctacccgctggcgcctctccaggaaggcatcttcttccaccacctcctggatgcgggagagggcgatgtctacatccagcccctcgtcctgcggtttgactcccgggagcggctcgatgtcttcaccggcgctttgcagcgtgtggtggaccggcacgacatcctgcgcacggcattcgtgtggcaggacctgccccagccggtgcaggtcgtcctgcgcacggcatcgctgcccgtggaaacggttgttctgggcaccgcaggaaacccggcggagcagctggtggcggcctgcccggcgagcatggacatcggtcaggcgcccctcatccgggtgttcatcgcgcaggagcctgacacgggtgcctggcatgccctgatccagacgcaccacctgatcctcgaccacaccaccatggacgtcctgatgggcgagatccaggcggtggtggcggggcgggaggagagccttccggagccgctgccgttccgtgatttcgtcgcgcaggcccggctcggtactccccgcgaggagcacgagcggttctttgccgagttgctgggagacgtcacggagccgaccgctccgtacgacctggtcgacgtgcagggtgatggcgccgatgtcgccgagcggaagctctccctcgatgccggcctcgccacccggctccgctcccgggcccgtgcgctcggggtcagcccggctaccgtcttccacctggcctgggcgcgcctgatcggaaccctgaccgggcgtgacgacgtggtcttcggcacggtcctgttcgggcggatgaacgccggtgccggcgccgaccgcgtccccggcctgttcatcaacacccttcccgtacgcacacgcctgcacgaggacaccgtcggcgacgcgctgcagaccatgcaccgtcacctggcggaccttctcgcccatgagcacgcgccgctcgcgctcgcgcagcaggccagtggactgccgccgcagacaccgatgttcacctcactgctgaactaccggcactccgccgaggcagccaccgggtccagcaacgcgccggagggggtcgagctgctgtacgcgcgcgagcggacgaactatccgctgggcgtgtacatcgatgacacgacggtcggcttccgtctgaccgttcaggccgccgaccccatcgacgccgatgctgtcgcccgctggatgcgcaccgtcaccgagaacatcgtccaggccctggaatcggccccggacacaccgatgcggcgcgtggaagcgctggacgacgccgaccgccaccgtctgctggtggagtggagcggcggcggtgccgagcccgtatccgcatccgcatccgagcccgcatccgccacgctggtggagttgttcgaggcgcaggcggctggtgcgccg

SEQ ID NO: 107 - protein, 465 aa, Streptomyces sp. strain ECO-38
ADIYPLAPLQEGIFFHHMLHADSGSDVYVLPSVLRFDSRERLDAFTGALQRVVDRHDILRTAFVWQDLPQPVQVVLRTAPIPVQHVHWEPQPSVTFADQLVAASPASMDIGQAPLIRLYTAQDPDTGAWHALIQVHHLIQDHTTLDILLAEIQAMVAGREESLPEPLPFRDFVAQARLGTPREEHERFFAELLGDVTEPTAPFGVVDVQGDGGAVRETVLPVDADLAGRLRMQARALGVSPATVFHLAWARLAGALSGRDDVVFGTVLFGRMNAGAGADRVPGLFINTLPVRARLDEQSVADALTGMHRHLADLLAHEHAPLALAQQASGLPPQTPLFTSLLNYRHSAEAGTASGNTLEGVESLYSRERTNYPLGVFIDDSTVGFHFTVQAADPIDAGAVARWMHTVTENIVRALESAPRTPVRRVEVLGDADRRRLLVEWNETARPVAGATLVELFEARVAGAP

SEQ ID NO: 108 - DNA, 1395 bp, Streptomyces sp strain ECO-38
gcggacatctacccgctggcgcctctccaggaaggcatcttcttccaccacatgctgcatgcggattccggctcggacgtgtatgtgctgccgtccgttctgcggttcgactcccgggagcggctcgatgccttcaccggtgctttgcagcgtgtggtggaccggcatgacatcctgcgcacggcttttgtgtggcaggacctgcctcagccggtgcaggtcgtcctgcgcacggcaccgatcccggtccagcacgtccactgggagccgcagccgtcggtgaccttcgcggaccagttggtggcggccagcccggcgagcatggacatcggtcaggcgcccctcatccgcttgtacaccgcgcaggacccggacaccggtgcctggcatgccctgatccaggtgcatcacctgatacaggaccacaccaccctggacatcctgctggccgagatccaggcgatggtggcggggcgggaggagagccttccggagccgctgccgttccgtgactttgttgcgcaggcccggctcggtacccctcgcgaggagcacgagcggttcttcgccgagttgctgggagatgtcacggagccgaccgcgccgttcggtgtggtggatgtgcagggtgacggcggtgcggtccgggagaccgtgctgccggtggacgcggatctcgcggggcggctgcggatgcaggcccgtgcgctcggggtcagcccggccaccgtcttccacctggcctgggctcgtctggccggagcgctgtccgggcgtgacgacgtggtcttcggtacggtcctgttcgggcggatgaacgccggtgccggcgctgaccgcgtccccggcctgttcatcaacacccttccggtgcgcgcacgtctggacgagcagtcggtggccgacgcgctgaccggtatgcaccgtcacctggcggacctcctcgcccatgagcacgcgccgctcgcgctcgcgcagcaggccagtggactgccgccgcagacgccgctgttcacctcactgctgaactaccggcactccgccgaagcaggcacggcctccggcaacacgctggagggagtcgagtcgttgtactcgcgcgagcggacgaactatccgctgggcgtgttcatcgacgactcgacggtcggcttccatttcaccgttcaggccgccgaccccatcgatgccggtgctgtggcccggtggatgcatacggtcacggagaacatcgtccgggccttggagtctgctccgcgcacgccggtgcggcgggtggaggtgctgggtgatgccgaccgtcggcgtctgctggtggagtggaatgagacggcccggccggtggcgggtgcgacgttggtggagttgttcgaggcgcgggtggcgggtgcgccg

SEQ ID NO: 109 - protein, 463 aa, Streptomyces sp strain ECO-38
ADIYPLAPLQEGIFFHHLLDAGEGDVYIQPLVLRFDSRERLDVFTGALQRVVDRHDILRTAFVWQDLPQPVQVVLRTAPIPVRHVHWEPQPSVTFAEQLVAASPASMDIGQAPLIRIYIAQEPDTGAWHALIQTHHLILDHTTMDILMAEIQAVVAGREESLPEPLPFRDFVAQARLGTPREEHERFFAELLGDVTEPTAPYDLVDVQGDGADVAERKLSLDAGLATRLRSRARALGVSPATVFHLAWARLIGTLTGRDDVVFGTVLFGRMNAGAGADRVPGLFMNTLPVRARLHGATVGDALQAMHRHLADLLAHEHAPLALAQQASGLPPQTPLFTSLLNYRYTGEGTTAAAAPAGIESLYGRERTNYPLSVHVDDTSAGFTLTVQTAAPIAPDAIAHQLHTVTENIVATLEAASPTPMDAIGVMEEAERERVLVKWNDTAHALPDATVAELFEAQVTRTP

SEQ ID NO: 110 - DNA, 1389 bp, Streptomyces sp strain ECO-38
gcggacatctacccgctggcgccgctccaggaaggcatcttcttccaccacctcctggacgcgggagagggcgatgtctacatccagcccctcgtcctgcggttcgactcccgggagcggctcgatgtcttcaccggtgctttgcagcgtgtggtggaccggcacgacatccttcgcacggcattcgtgtggcaggacctgccccagccggtgcaggtcgtcctgcgcacggcaccaatcccggtccgacacgtccactgggagccgcagccgtcggtgaccttcgccgaacaactggtagcggctagcccggcgagcatggacatcggacaggcgcccctcatccgcatatacatcgcgcaggagccggacaccggtgcctggcatgccctgatccagacgcaccacctgatcctcgaccacaccaccatggacatcttgatggcggagatccaggcggtggtggcggggcgggaggagagccttccggagccgctgccgttccgtgatttcgtcgcgcaggcccggctcggtactccccgcgaggagcacgagcggttctttgccgagttgctgggagacgtcacggagccgaccgctccgtacgacctggtcgacgtgcagggtgatggcgccgatgtcgccgagcggaagctctccctcgatgccggcctcgccacccggctccgctcccgggcccgtgcgctcggggtcagcccggctaccgtcttccacctggcctgggcgcgcctgatcggaaccctgaccgggcgtgacgacgtggtcttcggcacggtcctgttcgggcggatgaacgccggtgccggcgccgaccgcgtccccggcctgttcatgaacacccttcccgtgcgggcgcgcctgcacggggccaccgtcggcgacgcgctgcaggccatgcaccgtcacctggcggaccttctcgcccacgagcacgcgccgctcgcgctcgcgcagcaggccagtggactgccgccgcagacgccgctgttcacctccctcctcaactaccgctacaccggcgagggcaccaccgcggccgccgctcctgccgggatcgagtcgctctacggccgggagcgcaccaactaccccctgtccgtgcacgtggacgacacctcggccggcttcactctgacggtacagacggccgccccgatcgcccccgacgcgatcgcgcatcagttgcacacggtcaccgagaacatcgtcgcgacccttgaggcggcctctccgactccgatggacgcaatcggggtcatggaggaagccgagcgggaacgggtgctggtgaagtggaacgacactgcgcatgcgctgccggacgccacagtggcggagctgttcgaggcgcaggtgacccggacgccg

SEQ ID NO: 111 - protein, 466 aa, Streptomyces sp strain ECO-59
ADVYPLAPLQEGLLFHHLLADGGDDAYVTPVLLEFDSRARLDGFVEALRQVIDRHDIFRTSVVWQGLDEPVQVVWRRAELPVTEVVLDPDGPETVEQLLSIGGLSMDLGRAPLIDVHVAAKPGSDHFLALLRMHHMVQDHTTLEVLLGEIRAFLDGRGDRLGEPLPFREFVAQARGGVDRAEHEEFFAGLLGDVSEPTAPYGLVDVRGDGSGSVETRAVVEAAVVGRLRVVARRLGVSAATVLHVAWARVLAAVSGREDVVFGTVLFGRMNAGAGADRVPGPFMNTLPVRARVDGVGVLDAVTAMRGRLAGLLEHEHAPLALAQQASAVPADAPLFTALLNYRHNIGGRLEGTVLDGTDLLLVRERTNFPLWVAVDDNGDTMELVVDAVASVDPEAVAGALSTAVEGLVSALEAALEGGPEPWLSTVEVIGAAERDRVLVEWNDTAVEVGAATLPELFEAHVVRSP

SEQ ID NO: 112 - DNA, 1398 bp, Streptomyces sp strain ECO-59
gcggatgtgtatccgctggcgccgctgcaggagggtctgctcttccaccatctcctcgcggacggcggagacgacgcatatgtgacgcctgtgctcctggagttcgactcacgggcgcggctggacgggtttgttgaggccttgcggcaggtgatcgatcggcatgacatcttccggacgagtgtggtgtggcaggggctggatgagccggtgcaggtggtgtggcgccgggcggagctgccggtgaccgaggtggtcctggatccggacgggccggagacggtggagcagttgctgtccatcggcggtctgtcgatggatctgggccgggcgccgttgatcgatgtgcacgtggctgccaagccgggcagcgaccacttcctggcgttgctgcgcatgcatcacatggtgcaggaccacaccacgctggaggtgctgctgggtgagatacgggcgttcctggacggtcgtggtgaccgtctgggtgagccgttgccgttccgggagtttgtggcgcaggcacgtggtggggtggaccgggcggagcatgaggagttcttcgctgggttgctgggtgatgtgagtgagccgacggcgccgtacgggctggtggatgtccgtggggatgggtcgggctcggtggagacgcgggcggtggtggaggccgctgtggtgggccggctgcgtgtggtggcgcggcggttgggggtcagtgcggcgacggtgctgcatgtggcgtgggcgcgggtgctggcggcggtcagtggccgtgaggacgtggtgttcggcacggtgctgttcggccggatgaacgcgggtgcgggtgcggaccgggtgccggggccgttcatgaacacgctgccggtgcgggcacgggtcgatggtgtgggcgtgctggacgcggtgacggccatgcgtggccggctggcgggactgctggagcacgagcatgcaccactggccctggcccagcaggccagcgcagtgcccgccgacgcaccgctgttcaccgcgctgctcaactaccgccacaacatcggcggacgcctggagggcactgtcctcgatggcaccgacctgctactcgtccgtgaacgcaccaacttcccgttgtgggtggcggttgatgacaacggggacacgatggagctggtcgtggatgccgtggcttcggtggatccggaggctgtggccggggcgctgagtaccgcggtggagggcttggtgtcggctctggaggcggcactggagggcggtccggagccctggctgagcacggtcgaggtgatcggcgccgcggagcgggaccgggtgctggtggagtggaacgacaccgcggtggaggtgggtgctgcgacgcttccggagttgttcgaggcgcatgtggtgcggtcgccg

SEQ ID NO: 113 - protein, 466 aa, Streptomyces sp strain ECO-59
ADVYPLAPLQEGLLFHHLLADGGDDAYVIPTVLEFDSRARLDGFVGALQQVIARHDIYRTSVVWQGLGEPVQVVWRRAELPVTEVVLDPDGPEAVEQLLSIGGLSMDLGRAPLIDVHVARAPGGEVWLGLVRVHHMVQDHMGLEVLLGEIRAFLDGRGDRLGEPLRFREFVAQARGGVERAEHEEFFAGLLGDVSEPTAPYGLVDVRGDGSGSVETRAVVEAAVVGRLRVVARRLGVSPATVLHVAWARVLAAVSGREDVVFGTVLFGRMNAGAGADRVPGPFMNTLPVRARVDGVGVLDAVTAMRGRLAGLLEHEHAPLALAQQASAVPADAPLFTALLNYRHNIGGRLEGTVLDGTDLLLVRERTNFPLWVAVDDNGDTMELVVDAVASVDPEAVAGALSTAVEGLVSALEAALEGGPEPWLSTVEVLDSVERDRVLVEWNDTAVEVGAATLPELFEAQVVRSP

SEQ ID NO: 114 - DNA, 1398 bp, Streptomyces sp strain ECO-59
gcggatgtgtatccgctggcgccgctgcaggagggtctgctcttccaccatctcctcgcggacggcggagacgacgcgtatgtgatccccacggtgctggagttcgactcgcgggcccggctggacgggttcgtgggcgcgttgcagcaggtgattgctcggcatgacatctatcgcacgagtgtggtgtggcaggggctgggtgagccggtgcaggtggtgtggcgccgggcggagctgccggtgaccgaggtggtcctggatccggacgggccggaggcggtggagcagttgctgtccatcggcggtctgtcgatggatctgggccgggcgccgttgattgatgtgcatgtggcccgggcgccggggggtgaggtgtggctggggctggtgcgggtgcatcacatggtgcaggaccacatggggctggaggtgctgctgggggagatacgggcgttcctggacggtcgtggtgaccggctgggtgagccgttgcggttccgggagtttgtggcgcaggcacgtggtggggtggagcgggcggagcatgaggagttcttcgctgggttgctgggtgatgtgagtgagccgacggcgccgtacgggctggtggatgtccgtggggatgggtcgggctcggtggagacgcgggcggtggtggaggccgctgtggtgggccggctgcgtgtggtggcgcggcggttgggggtcagcccggcgacggtgctgcatgtggcgtgggcgcgggtgctggcggcggtcagtggccgtgaggacgtggtgttcggcacggtgctgttcggccggatgaacgcgggtgcgggtgcggaccgggtgccggggccgttcatgaacacgctgccggtgcgggcacgggtcgatggtgtgggcgtgctggacgcggtgacggccatgcgtggccggctggcgggactgctggagcacgagcatgcaccactggccctggcacagcaggccagcgcagtgcccgccgacgcaccgctgttcaccgcgctgctcaactaccgccacaacatcggcggacgcctggagggcactgtcctcgatggcaccgacctgctactcgtccgtgaacgcaccaacttcccgttgtgggtggcggttgatgacaacggggacacgatggagctggtcgtggatgccgtggcttcggtggatccggaggctgtggccggggcgctgagtaccgcggtggagggcttggtgtcggctctggaggcggcactggagggcggtccggagccctggctgagcacggtcgaggtgctggattccgtggagcgggaccgggtgctggtggagtggaacgacaccgcggtggaggtgggtgctgcgacgcttccggagttgttcgaggcgcaggtggtgcggtcgccg

SEQ ID NO: 115 - protein, 464 aa, Streptomyces virididaciens strain NRRL ISP-5239
ADIYPLAPLQEGILFHHLTAAEGRDVYVLPVVLAFDGRERLDGFLTALRSVVDRHDVLRTAVLWEGLPEPVQVVVRHVTVPVEEVDLTAVPAADGAAVHPLLAACPTTMDLGSAPLLRGHVAAEPGTDRWLLLLQQHHLVVDNTALAVLLAEVQARLAGEADRLPPPAPYRDFVAQTRLGCQDADHERYFAGLLGDVAEPTAPFGVLDVRGDAGGHAEATFLVEAREAERLRSLASRHGVSAAALFHLAWARVVAATSNQDDVVFGTVLFGRAAGGGDVPGLFINTLPVRQATGAVAVADALREMQTQLAALLAHECAPLSLVQRTSAVEGTAPLFTALLNYRRVQGFALRAGTVEALPGVELVHLEDRTNYPLALTVDDRVDAFTCSVQAVAPIDGAAVAALVEQTLASLADALDHAPRTAVGDLDVLGSEERQGLLTQGSAGGPVAPALLPELVAAQAARTP

SEQ ID NO: 116 - DNA, 1392 bp, Streptomyces viridifaciens strain NRRL ISP-5239
gcggacatctacccgctggcacccctccaggagggcatcctcttccaccacctgacggccgccgagggccgcgacgtctacgtcctgcccgtggtgctcgccttcgacggtcgcgagcggctcgacggcttcctcaccgcgctgcggtccgtcgtcgaccgccacgacgtactgcgcaccgccgtgctgtgggagggcctgcccgagccggtccaggtcgtcgtacgccacgtcaccgtcccggtcgaggaggtcgacctcacggcggtcccggctgccgacggcgccgcggtgcacccgctcctcgccgcctgcccgacgaccatggacctcggcagcgccccgctgctacgcggccacgtcgcagccgagcccggcaccgaccgatggctgctgctgctccagcagcaccacctcgtcgtcgacaacaccgccctggccgtgctcctcgccgaggtccaggcgcgcctcgcgggcgaggcggaccgcctgccgccgcccgcgccctaccgggacttcgtcgctcagacccgcctgggctgccaggacgcggaccacgaacgctacttcgccgggctgctcggcgacgtggccgagcccaccgcgccgttcggcgtgctggacgtgcgcggggatgcgggcggccacgccgaggcgacgttcctcgtcgaggccagggaggccgagcgactgcgctccctggcgagccgccacggtgtcagcgccgccgcgctgttccacctcgcctgggcccgggtcgtcgcggccacctcgaaccaggacgacgtcgtcttcggcaccgtcctcttcggccgcgcggccggtggcggcgacgttccggggctcttcatcaacaccctgcccgtccgccaggccaccggcgccgtggccgtggccgacgccctgcgggagatgcagacccagctcgccgccctgctggcccacgagtgtgcgccgctgtcgctggtccagcggacctccgccgtcgagggcaccgcgcctctcttcaccgcgctgctcaactaccgccgcgtacagggcttcgccctgcgcgcgggcacggtcgaggcgttgcccggtgtcgagctggtccacctggaggaccgcaccaactacccgctcgcgctgaccgtcgacgaccgcgtcgacgcgttcacctgctccgtccaggccgtcgcgccgatcgatggggcggccgtcgcagccctggtcgaacagacgctcgcctcgctggccgacgcgctcgaccacgccccgcggaccgccgtcggcgacctcgacgtgctcggctcggaggagcgtcagggcctgctgacgcagggcagtgcaggcggtccggtcgcgcccgcgctgttgccggagctggtcgccgcgcaggcggcgcgcacgccg

SEQ ID NO: 117 - protein, 467 aa, Streptomyces viridifaciens strain NRRL ISP-5239
ADVYPLAPLQEGIFFHHLMAAGEGGRDMYVVPALLTFDSRERLDAFLGALQQVIDRHDILRTGVFWDGLAEPVQVVARHAQLPVETVNLSGPVEQAMGELQGACPPVMDLTRAPLLRAFVAAEPGGGRWLLALQRHHLVTDHTALDILLGEIRAVLAGEQDRLPAPLPFREFVAQARLGVSREEHERFFAGLLGDVAEPSAPFGLLDVRGDGSRVSEAEHVLAPELAARIRELSRVLGVSPATLFHVAFARVVAATANRDDVVFGTLLFGRMNAGSGADRVPGLFINTLPVRLDTAAHTVADAVGAMRGQLADLLVHEHAPLALAQRASGITAPAPLFTALLNYRHSQLVDEEVEGSGLAGVEHAEHGKDRTNYPLTVAVDDFGSRLQLSVQALAPIDPELVCALALTTLTGLVDALETAPETPLAAVDVLDEDRRRTVLHTWNDTAAGISAVSLTDLFEAQAARTP

SEQ ID NO: 118 - DNA, 1401 bp, Streptomyces viridifaciens strain NRRL ISP-5239
gccgacgtctaccccctcgcccccctccaggaaggcatcttcttccaccacctgatggctgccggcgagggcggacgtgacatgtacgtcgttccggccctgctcaccttcgactcgcgcgagcgcctcgacgccttcctcggcgcgctccaacaggtgatcgaccgtcacgacatcctgcgtacgggcgtgttctgggacggcctggccgagccggtgcaggtcgtcgcgcgccatgcccaactgccggtcgagaccgtcaacttgtcaggccccgtcgagcaggcgatgggcgagctccagggggcctgcccgcccgtgatggacctgacccgggcgccgctgctgcgtgccttcgtcgcggccgaacccggcggcgggcgctggctgctggcgctccagcgccaccacctggtcaccgaccacacggcgctggacatcctcctcggcgagatccgggccgtgctggcgggggagcaggaccgcctgcccgcgccgctgccgttccgcgagttcgttgcccaggcccggctcggcgtctcccgcgaggagcatgagcggttcttcgcgggcctgctgggtgacgtggccgaaccgtccgcgccgttcgggctgctggacgtgcgaggtgacggcagccgggtcagtgaggcggagcacgtgctcgcgccggaactggccgctcgtatcagggagctgtcgcgggtgctgggggtgagcccggcgacgctgttccacgtcgccttcgcccgcgtcgtggccgcgaccgccaaccgcgacgacgtggtgttcggcacgctgctcttcggccggatgaacgcgggcagcggtgccgaccgggtgccggggctgttcatcaacaccctgcccgttcgcctcgacaccgccgcccacaccgtcgccgacgcggtcggcgcgatgcgcggacagctcgccgacctcctcgtccacgagcacgccccgctcgcgctggcccagcgagccagcggcatcaccgcccccgccccgctgttcaccgccctgctcaactaccggcacagccagttggtggacgaggaggtcgagggcagcggcctggccggcgtcgagcacgccgagcacggcaaggaccgcaccaactacccgctcacggtcgcggtcgacgacttcggctcgcgactccagctgtccgtccaggcgctcgcgccgatcgaccccgagctggtctgcgcgctcgcgctcaccacgctgacgggcctggtcgacgcgctggagacggccccggagacgcccctcgccgccgttgacgtcctcgacgaggaccgtcgccggacggtgctgcacacctggaacgacacggcggccggcatctccgccgtcagcctcactgacctgttcgaggcccaggcggcccgcactccg

SEQ ID NO: 119 - protein, 461 aa, Streptomyces viridifaciens strain NRRL ISP-5239
ADVYPLAPLQEGIFFHHLMAVDGEDPYVLPSLLTFDSRERLDAFLGALRFVIDRHDILRTAFFWQGLPEPVQVVARRAELPVQTVELSGEQDPVAELHAACPARMDIGAAPLLRAFFAAEVGTGRWLLVLQRHHLINDHTALDVLMSEIRAVLEGAADHLPAPLPFRDLVAQARLGISREDHERYFAGVLGDVAEPSAPFGLLDVRGDGSTVDRARLPLAPELAARVREQARVLGVSPATLFHVAFARVVAATANRDDVVFGTLLFGRMNAGSGADRVPGLFINTLPVRLDTAAHTVADAVGAMRGQLADLLVHEHAPLALAQRASGITAPAPLFTALLNYRHGRGHGDEPAADLGIGLEHGEDRTNYPLTVSVDDHGTALNLSVIAVAPIDAALVCTLLGTAVEGLVTALETAPHTSLRAVDVLDDALRRRMLTEWTKHPVRMPVSTVPELFEAQAARTP

SEQ ID NO: 120 - DNA, 1383 bp, Streptomyces viridifaciens strain NRRL ISP-5239
gccgacgtctaccccctcgcccccctccaggaaggcatcttcttccaccacctgatggctgtcgacggcgaggacccctacgtcctgccgtcgctgctcaccttcgactcgcgcgagcgcctcgacgccttcctcggcgcactgcggttcgtgatcgaccgacacgacatcctccgtaccgcgttcttctggcagggcctgcccgagccggtacaggtcgtcgcccgccgggccgagctgcccgtccagacggtcgagctgagcggggagcaggatccggtcgccgaactccacgccgcctgcccggcccgcatggacatcggcgccgccccgctgctgcgcgcgttcttcgccgccgaagtcggcaccgggcgctggctgctggtgctccagcggcaccacctcatcaacgaccacaccgccctggacgtgctgatgtccgagatccgggcggtcctggagggcgcggccgaccacctgccggcgccgctgccgttccgcgacttggtcgcgcaggcccggctcggcatctcccgtgaggaccacgagcgctacttcgccggcgtgctgggcgacgtcgcggagccgtccgcgccgttcgggctgctggacgtgcgcggcgacggcagcaccgtggaccgcgcccgccttccgctcgcgcccgaactggcggcccgcgtcagggagcaggcacgtgtgctgggggtgagcccggcgacgctgttccacgtcgcgttcgcccgcgtcgtggccgcgaccgccaaccgcgacgacgtggtgttcggcacgctgctcttcggccggatgaacgcgggcagcggtgccgaccgggtgccagggctgttcatcaacaccctgcccgttcgcctcgacaccgccgcccacaccgtcgccgacgcggtcggcgcgatgcgcggacagctcgccgacctcctcgtccacgagcacgccccgctcgcgctggcccagcgagccagcggcatcaccgcccccgcaccgctgttcaccgccctgctcaactaccgccacgggcgtggccacggcgacgagcccgccgccgacctcggcatcggcctcgaacacggcgaggaccgcaccaactacccgctcaccgtctccgtggacgaccacggcaccgcgctcaacctttccgtgatcgccgtcgcgcccatcgacgcggcactggtctgcacgctgctcggcaccgcggtggagggcctggtcaccgcactggagacggcgccgcacacctccctgcgggccgtggacgtgctcgacgacgcgctgcggcggcgcatgctgacggagtggaccaagcaccccgtccgcatgcccgtctcgaccgttcccgagctcttcgaggcgcaggccgcgcgtacaccc

SEQ ID NO: 121 - protein, 464 aa, Streptomyces viridifaciens strain NRRL ISP-5239
ADIYPLAPLQEGIFFHHLMNAGGRDVYVLPTVLSFDSLARLHGFLGALQTVVDRHDVLRTAVVWEGLAEPVQVVARTAEIPVTVLELTGSDDPVVELHAACPGSMDLGAAPLMCAHIAADPSREGRWLMLLQRHHLTTDHTAMEVLLAEIQAVLAGEGDGLPVPIPFRNFVAQARLRVTREEHERFFAELLGDVTEPTAPFGLLDVRGDAAQVAEARLHLADPLAERLRGQARRLGVSPATVFHLAWARVAAATSNREDVVFGTVLFGRMNGGSGADRVPGLFINTLPVRARTAGVSVTDALAAMRGQLADLLVHEHAPLTLAQRASGIGGTAPLFTSLLNYRHSAGVEEPARPAAPEGIELLMSQERTNYPLTVSVDDLGAGFRLTAQALAPIDAERVCRLLHTTLDALVDALENEPGTALGTVPVLDGAEARQLLDAGNGTPRERLLGSVPELFAAQVERTP

SEQ ID NO: 122 - DNA, 1392 bp, Streptomyces viridifaciens strain NRRL ISP-5239
gccgacatctacccgctggcgccgctccaggaggggatcttcttccaccatctgatgaacgcgggcgggcgcgacgtgtacgtgctcccgacggtcctctcgttcgactccctcgcccggctgcacggcttcctcggcgcgctgcagacggtcgtcgaccgccacgacgtgctgcgcacggcggtcgtgtgggagggcctggccgagccggtgcaggtcgtcgcccgaacggcggagatccccgtcacggtcctcgaactcaccggctccgacgacccggtggtcgagctgcacgccgcctgtcccgggtccatggacctcggcgccgcgccgctcatgtgcgctcacatcgccgccgacccgtcccgggaggggcgctggctgatgctgctgcagcgccaccacctcaccaccgaccacaccgccatggaggtcctgctcgccgagatccaggcggtcctggcgggggagggcgacggccttccggtcccgattccgttccgcaacttcgtcgcccaggcccgcctgcgggtcacccgcgaggagcacgagcggttcttcgccgaactgctcggcgacgtcaccgagccgaccgccccgttcggcctgctcgacgtgcgcggcgacgcggcccaggtcgccgaggcccggctgcacctcgccgacccgctcgccgagcggctgcgcggccaggcccgccgcctcggcgtcagcccggcgacggtgttccacctcgcctgggcccgcgtggccgcagcgacctccaaccgcgaggacgtggtcttcggcacggtgctcttcggccgtatgaacggcggctccggcgcggaccgggtcccggggctcttcatcaacaccctccccgtccgggcccgcaccgcgggcgtcagcgtcaccgacgcgctcgcggcgatgcgcgggcagctcgcggacctgctcgtgcacgagcacgcgccgctcaccctcgcgcagcgcgcgagcggcatcggcggcaccgctccgctgttcacctcgctgctgaactaccgtcacagcgcaggcgtggaggagccggctcgtccggcggcaccggagggcatcgagctgctcatgagccaggagcgcaccaactacccgctcaccgtctccgtcgacgacctcggcgcgggcttccggctcacggcccaggccctcgccccgatcgatgcggagcgggtctgccgactgctgcacacgactctcgacgcgctggtcgacgccctggagaacgagcccggcacggcgctcggcacggtgccggtccttgacggggccgaggcgcggcagctcctggacgcagggaacggtaccccgcgcgagcgcctcctcggctccgtgcccgagctattcgccgcccaggtggaacgcacgccc

SEQ ID NO: 123 - protein, 478 aa, Actinomadura sp strain ATCC 39334
ADVYPLAPLQEGIFFHHLLEAGTGSDTYVLPMVLEFDGRERLDAFVGALQQVVDRHDVFRTGVVWEGLREPVQVVWRKAKLPVHEVMLIAQAEDPVAELVAAGGLMMDLGKAPLLDVHVAQRPGTGMWLALIRQHHLVQDHTAMDVMVDEVRAFLSGRGAELPEPPLYREFVAQARGGVSEAEHERFFGELLAGVSEPTLPFGVAEVHGDGSGAKRAVRQLDEHLAARLREVARRLGVSPATVTHVAYARTLAAISGRDDVVFGTVLFGRMQAGAGADRAAGLFINTLPARIQVGGVGVLQAITDTRQLLAGLLEHEHAPLAVAQRASSVPGDAPLFASIFNYRHTRSAPKRGAGAPAPAAPGAPSRGGMRSVFSREQSNYPLAVAVGDQGDGFDLVVDAVAPVEAHMVCGLLHTTIGSLVDALDALLDTDRDVLLDQVEVLSEDERERVLAAGHGAVVPVPGASVVELFEEQVRRSP

SEQ ID NO: 124 - DNA, 1434 bp, Actinomadura sp strain ATCC 39334
gctgacgtctatccgctggctccgctccaggagggcatcttcttccaccatctcttggaggcaggtaccggaagcgacacctatgtgcttccgatggtgttggagttcgacgggcgggagcgtctggacgcgttcgtgggcgcgttgcagcaggtggtggaccgtcacgacgtgttccgcacgggtgtggtgtgggaagggttgcgtgagccggtccaggtggtctggcgtaaggccaagctgcctgttcacgaggtcatgctgatcgcccaggccgaagatccggtggccgaactggtggccgccggcggcctgatgatggacctgggcaaggcgcccctgcttgatgtgcatgtcgcgcaacgcccgggcacgggcatgtggctggctttgatccggcagcaccacctggtccaggaccacaccgccatggacgtgatggtcgatgaggtgcgggcgttcctgagcgggcgtggtgcggagttgcccgagccgccgctgtatcgggagttcgtggcgcaggcccggggcggggtttccgaggccgagcatgagcggttcttcggggagttgctggcgggggtgagtgagccgacgttgccgttcggggtggcggaggtgcacggtgatggctctggcgcgaagcgcgcggtgcggcaattggacgagcatctcgcggcgcggttgcgcgaggtggcgcggcggctgggtgtcagcccggcgaccgtgacgcatgtggcgtacgcgcggacgctggcggcgatctcgggccgtgatgatgtggtgttcgggacggtgctgttcggccggatgcaagcaggcgccggcgccgaccgcgcggcaggcctcttcatcaacaccctgcccgcccgcatccaggtcggcggagtcggagtcctccaggcgatcaccgacacccgccaactcctggccggtctgctggagcatgaacacgctcctctggccgtcgctcaacgtgccagttcggtcccgggcgatgctccgctcttcgcctcgatcttcaactaccgtcacacccgttccgctcccaagcgcggcgccggtgctccggctcccgccgcccccggagctccatcccgcggcggcatgaggtcggtcttcagccgggagcagagcaactatccgctcgcggtcgcggtgggtgatcagggcgacggattcgatcttgtggtcgacgcggtcgctccggttgaagcgcacatggtgtgcgggctgctgcacaccacgatcggcagcctggtcgacgcgctggacgccctgctcgacaccgaccgggacgttctgttggaccaggtcgaagtgctgtcggaggacgagcgggaacgggtgctggccgctggacacggtgccgtggtgcccgtccccggcgcgtcggtggtggagctgttcgaggagcaggtgcggcggtccccg

SEQ ID NO: 125 - protein, 468 aa, Actinomadura sp strain ATCC 39334
ADVYPLAPLQEGIFFHHLLEAGTGSDTYVLPMVLEFDGRERLDAFVGALQQVVDRHDVFRTGVVWEGLREPVQMVWRKAKLPVQEVSLDPKAEDPVAELVAAGGSVMDLGRAPLMDVHVAQRPGTGMWLALIRQHHLVQDHTAMDVMVDEVRAFLTGRGAELPEPPLYREFVAQARGRVAAAEHERFFGEMLAGVSEPTLPFGVAEVRGDGSDVVRATRSLEPELAERLREVARRLGVSPATVTHVAYARTLAVISGRDDVVFGTVLFGRMQAGAGADRAAGLFINTLPARIQVGGVGVLQAIANTRQLLAGLLEHEHAPLTLAQRASAVPGDTPLFTSLFNYTYSRVQAAAPASRAGAGGIKPVFSQDRHNYPLAVAVRDRGDGFDLLVDASAPMQADVVCALFHTTIANLVHALDDDVEGDLSLSAVEVLPAGERERVLSAGVGEAVSVPGVSVVGLFEERVRWSP

SEQ ID NO: 126 - DNA, 1404 bp, Actinomadura sp strain ATCC 39334
gctgacgtctatccgctggctccgctccaggagggcatcttcttccaccatctcttggaggcaggtaccggaagcgacacttacgtgcttccgatggtgttggagttcgacgggcgggagcgtctggacgcgttcgtgggcgcgttgcagcaggtggtggaccgtcacgacgtgttccgcacaggtgtggtgtgggaagggctgcgtgagccggtccagatggtctggcgtaaggccaagctgcccgtccaggaagtgagccttgatcccaaggcggaggatccagtggccgagttggtggccgcgggtggttcggtgatggatctgggtcgggcgcctttgatggatgtgcatgtcgcgcaacgcccgggcacgggcatgtggctggctttgatccggcagcatcacctggtccaggaccacaccgccatggacgtgatggtcgatgaggtgcgggcgttcctgaccgggcgtggtgcggagctgcccgagccgccgctgtatcgggagttcgtggcccaggcccggggccgggtcgcggccgccgaacatgagcggttcttcggggagatgctggcgggggtgagcgagccgacgctgccgttcggggtggcggaggtgcgaggcgacggttctgacgtggtgcgtgccacccggtcactggagccggagttggctgagcggttgcgcgaggtggcgcggcggctgggtgtcagtccggcgacggtgacgcatgtggcgtatgcgcggacgctggcggtgatctcgggccgtgatgatgtggtgttcgggacggtgctgttcggccggatgcaagcaggcgccggcgccgaccgcgcggcaggcctcttcatcaacaccctgcccgcccgcatccaggtcggcggagtcggagtcctccaggccatcgccaacacccgccaactcctggccggtctcctggaacatgagcatgccccactcaccctggcccagcgcgcaagtgcggttccgggtgatacccccctgttcacctcgctcttcaattacacctacagccgcgtccaagctgccgcccccgcatcccgcgctggtgctggtggcataaagccggtcttcagccaggaccggcacaactaccccctcgccgtggcggtacgtgaccggggtgacggcttcgacctgctggtcgacgcgtccgcaccgatgcaggcggacgtggtgtgcgcgcttttccacaccacgatcgccaacctggtccacgccttggacgatgacgtcgaaggcgatctatccctgagcgcggtagaggtgctgccggcgggcgagcgagagcgggtgctgtcggcgggggtcggcgaggccgtgtccgtgccgggcgtgtcggtggtggggctgttcgaggagcgggtgcggtggtcgccg

SEQ ID NO: 127 - protein, 473 aa, Actinomadura sp strain ATCC 39334
ADVYPLAPLQEGIFFHHLLEAQGSDDVYVQPTVVEFDGREHLDAFMGALQQVVDRHDVFRTGVVWEGLREPVQVVWRKAKLPVHEVMLDAQAEDPVAELVAAGGLMMDLGKAPLLDVHVAQRPGTGTWLALIRQHHLVQDHTGLDVLLGEVRAFMAGRGAELPEPPLYREFVAQARGGVPVAEHERFFEEMLAGVSEPTLPFGVAEVRGDGTGAVRTVQRLDEHLAARLRDVARRLGVSPATVMHVAYARTLAVISGRDDVVFGTVLFGRMQAGAGADRAAGLFINTLPARIRVGGVGVLQAITDTRELLAGLLEHEHAPLALAQRASAVPGDTPLFTSLFNYTYAYSTTSAPRDATRSPGGPKRVFSQTRNNYPLTVGIEDLREGFGLVVDTVAPMESDAVCRLLQTAIANLVDTMDALLDGDVEGDPSLSAVEVLPADERERVLAVGTGEVVPRSGASVVGLFEERVRRSP

SEQ ID NO: 128 - DNA, 1419 bp, Actinomadura sp strain ATCC 39334
gctgacgtctatccgctggccccactccaggaaggcatcttcttccaccacctcctggaagcccagggcagcgacgatgtctacgtgcaaccgaccgttgtcgagttcgacgggcgggagcatctggacgcgttcatgggcgcgttgcagcaggtcgtcgaccgtcacgacgtgttccgcacaggtgtggtgtgggaagggctgcgtgagccggtccaggtggtctggcgtaaggccaagctccctgttcacgaggtcatgctggacgcccaggccgaagatccggtggccgaactggtggccgccggcggcctgatgatggacctgggcaaggcgcccctgcttgatgtgcatgtcgcgcaacgcccgggcactggcacgtggctggctttgatccggcagcaccacctcgtccaggaccacaccggcctggacgtgttgctgggtgaggtgcgggcgttcatggcgggacggggcgcggagctgcccgagccgccgctgtaccgggagttcgtggcgcaggcccggggcggggttccggtcgccgagcatgagcgcttcttcgaggagatgctggcgggggtgagcgagccgaccctcccgttcggggtggcggaggttcgtggcgacgggaccggagcggtccgcaccgtgcagcggctggacgagcatctcgcggcgcggttgcgcgacgtggcgcggcggctgggtgtcagcccagcgaccgtgatgcacgtggcgtatgcgcggacgctggcggtgatctcgggccgtgatgatgtggtgttcgggacggtgctgttcggccggatgcaagcaggcgccggcgccgaccgcgcggcaggcctcttcatcaacaccctgcccgcccgcatccgagtcggcggagtcggagtcctccaggccatcaccgacacccgcgaactgctcgctggcctgctggaacacgagcacgccccactcgccctggcccagcgcgcgagcgcagtaccgggcgacaccccgctgttcacctcgctcttcaactacacctacgcgtacagcacgaccagtgcgcccagggatgcgacgcggagccccggcggccccaagagggtcttctcgcagacacgcaacaactatccgctgaccgtcgggatcgaggatctccgcgagggattcggcctggtggtcgacacggtcgctccgatggagtcggacgcggtgtgccgactgctgcagaccgcgatcgccaacctggtcgacaccatggacgcgctcctggacggcgacgtcgaaggcgatccctccctgagtgcggtagaggtgctgccggcggacgagcgggagcgggtgctggctgtggggaccggcgaggtggtgccgcggtcgggtgcttcggtggtggggttgttcgaggagcgggtgcggcgttcgccg

SEQ ID NO: 129 - protein, 471 aa, Actinomadura sp strain ATCC 39334
ADVYRLAPLQEGIFFHYLLGSDGDSDAYVLPLVLEFDGRERLHAFLDALQQVVDRHDVFRTGVVWEGLREPVQVVWRKAKLPVHEVSLDPEAKDPAAELVNAGGLVMDLGRAPLMDVHVAQRPDTGQWLALIRIHQLVRDHTGLDVLLGEVRAFMAGRGAELPEPPLYREFVAQARGGVSEAEHERFFGELLAGVSEPTLPFGVAEVHGDGSGAKRAVRQLDEHLAARLREVARRLGVSPATVTHVAYARTLAAISGRGDVVFGTVLFGRMQAGAGADRAAGLFINTLPARIQVGGVGVLQAITDTRQLLARLLEHEHAPLAVAQRASAVPGDTPLFASIFNYRYTSPAPASGGLGEGMKLVSSRERSNYPLAVAVSDSGVGFDLLVDAVAPVDADVVCGLFHTTIGHLVDALDTHQDMSAAMETVAPLSTVAVLSEVERERVLAAGTGEVAPLPGTSVMELFQRQVLRSP

SEQ ID NO: 130 - DNA, 1413 bp, Actinomadura sp strain ATCC 39334
gccgatgtctatcggctggccccgctccaggaaggcatcttcttccactatctgctgggctcggatggggacagcgacgcgtatgtgcttcccctggtgctggagttcgacggacgtgagcgtctgcacgcgttcttggatgcgttgcagcaggtggtggaccgtcacgacgtgttccgcacgggcgtggtgtgggaagggctgcgtgagccggtccaggtggtctggcgcaaagccaagctgcccgtccatgaagtgagccttgatcccgaggcgaaagatccggcggccgagttggtgaacgccggtggcctggtgatggatctggggcgcgcgcctctgatggatgtgcatgtcgcgcagcgtccggacacgggtcagtggctggccctgatccggatccatcagctggtccgtgaccacactggcctggacgtgttgctgggtgaggtgcgggcgttcatggcggggcgtggtgcggagttgcccgagccgccgctgtatcgggagttcgtggcgcaggcccggggcggggtttccgaggccgagcatgagcggttcttcggggagttgctggcgggggtgagtgagccgacgttgccgttcggggtggcggaggtgcacggtgatggctctggcgcgaagcgcgcggtgcggcagttggacgagcatctcgcggcgcggttgcgtgaggtggcgcggcggttgggtgtcagcccggcgacggtgacgcatgtggcgtatgcgcggacgctggcggcgatctcgggccgtggtgatgtggtgttcgggacggtgctgttcggccggatgcaagcaggcgccggcgccgaccgcgcggcaggtctctttatcaacaccctgcccgcccgcatccaggttggcggagtcggagtcctccaggccatcaccgacacccgccaactgctcgcccgtctcctggaacacgagcacgccccactcgccgtcgcccagcgtgcgagcgcagtaccgggcgacaccccgctgttcgcctccatcttcaactaccgatacaccagcccggctcccgcctccggtgggctgggcgaaggaatgaagctggtctccagcagggagcgcagcaactacccgcttgcggtcgccgtgagcgactcgggtgttggattcgatctgctggtggacgcggtcgccccggtggacgcggacgtcgtgtgcgggttgttccacaccacgatcggccacctggtcgacgcgctggacacccaccaggacatgagcgctgcgatggagaccgtcgctcctctgtccacggtcgccgtgctgtcagaggtcgagcgggagcgggtgctcgctgcggggaccggcgaagtggccccgctgccgggcacgtcggtgatggagctcttccagaggcaggtactgcggtcgccg

SEQ ID NO: 131 - protein, 459 aa, Micromonospora chersina strain ATCC 53710
ADVYPLAPLQEGLFFHHLMADRDGTDVYVTPTVLRCDSRERLDAFLAALQQVVDRNDVYRTAIVHEGLREAVQVVVRHADLPVHEVTVDPADPVQALLAAAGGWMELRRAPLVDAYVAAEPGGDGWLVLVRIHHLVQDHTALEVLLHELHAYLEGRGDTLPAPVPFREFVAQARLGVSRAAHERWFADLLGDVTETTAPYGLLDVHGDGAAYVQGRRTVEPALAARVRDLARTLGVSPATLFHLVWARVLGVLAGRDDVVFGTVLFGRMNAGAGADRVSGLFINTLPVRVGLRGTVGGALAALRDQLADLLVHEHAPLAVAQAASGVPGGSPLFTSIFNYRHNQVSAEEAGAALPGITVVSARDLTNYPVTVAVDDDGTGFDLVVEGPVDADGVCALLRTALAGIVAALTDRPDTPLDAVDVLDADERARLLTAWNPAPVPVADRTLPDLFDAQAARAP

SEQ ID NO: 132 - DNA, 1377 bp, Micromonospora chersina strain ATCC 53710
gccgacgtctacccgctcgccccgctccaggaggggctgttcttccaccacctcatggccgaccgggacggcaccgacgtctacgtcacgcccaccgtgctgcgctgcgacagccgcgagcggctcgacgccttcctggccgccctccagcaggtggtcgaccgcaacgacgtctaccgcaccgcgatcgtccacgaggggctgcgcgaggccgtccaggtcgtcgtccgccacgccgacctgcccgtccacgaggtcaccgtcgaccccgccgacccggtccaggcgctgctcgccgcggccggcggctggatggaactgcgccgcgcgccgcttgtcgacgcgtacgtggccgccgaacccggcggcgacggctggctcgtgctggtccgcatccaccacctcgtgcaggaccacaccgccctcgaggtgctgctgcacgaactgcacgcctacctggaggggcgcggcgacacgctgcccgccccggtgccgttccgcgagttcgtcgcccaggcccggctcggcgtgtcccgcgccgcgcacgagcgctggttcgccgacctgctcggcgacgtcaccgagaccaccgccccgtacgggctgctcgacgtgcacggcgacggcgccgcgtacgtccaggggcggcgcaccgtcgaaccggcgctcgccgcccgggtccgcgacctggcccgtaccctcggggtcagccccgccaccctgttccacctggtgtgggcgcgggtcctcggggtcctcgccggccgcgacgacgtggtcttcggcaccgtcctgttcggccggatgaacgcgggggccggcgccgaccgggtctccggcctgttcatcaacaccctgccggtgcgggtcggcctgcgcggcaccgtcggcggcgccctggcggcgctgcgggaccagctcgccgacctgctggtgcacgagcacgccccgctcgccgtcgcccaggcggccagcggcgtgcccggcggcagcccgctgttcacgtcgatcttcaactaccggcacaaccaggtcagcgccgaggaggccggcgccgccctgcccggcatcacggtggtctccgcccgcgacctcaccaactacccggtcaccgtcgccgtggacgacgacgggaccggcttcgacctggtcgtcgaggggcccgtcgacgccgacggggtgtgcgcgctgctgcggaccgccctggccgggatcgtcgccgcgctgaccgaccgaccggacaccccgctggacgccgtcgacgtcctcgacgccgacgagcgcgcccggctgctcaccgcctggaacccggcccccgtgccggtggcggaccgcaccctgccggacctcttcgacgcgcaggcggcccgggctccg

SEQ ID NO: 133 - protein, 460 aa, Micromonospora chersina strain ATCC 53710
ADVYPLAPLQEGIFFHHLMDDDTDVYALPYVLHLDTRDRLDAFLAALRHLVARNDIYRTSVVWDGLREPVQVVWRHADLPVEEATLDPAADPVEALLAAAGTRIDLGAAPLMRAHVAAAPDGGWLLLLRIHHLVQDHTTFDVVLDELRAVLTGRAEELPPPLPYREFVARARLGVSDEEHERYFADLLGDVTETTAPYGLTDTHHDGTTAVQVQLTADRALTARVRDLARAYAVSPATILHLAWARLLGTLAGRDDVVFGTILFGRMNAGAGADRAPGLFINTLPVRVRLARHGVADALTALRDQLADLLVHEHAPLALAQRASGVPAAAPLFTSLFNYRHNLPTDGHPGAGLDGVTAVLHRDNTNYPMVVSVDDDGTDFALVVEAVAPADPTRVGSLLLTCLDSLTAALEQAPQTPLGAVEVLDAAELRQVVEEWNATEAPVTDEPVPAAFARRVAADP

SEQ ID NO: 134 - DNA, 1380 bp, Micromonospora chersina strain ATCC 53710
gccgacgtctaccccctggccccgctccaggagggcatcttcttccaccacctgatggacgacgacaccgacgtctacgccctgccgtacgtgctgcacctcgacacccgcgaccgcctcgacgccttcctggccgcgctgcgccacctggtggcccgcaacgacatctaccgcacgagcgtcgtctgggacgggctgcgcgaaccggtccaggtggtgtggcgccacgccgacctgcccgtcgaggaggccaccctcgacccggccgccgacccggtcgaggcgctgctcgccgccgccggcacccggatcgacctcggcgccgccccgctgatgcgcgcccacgtggccgccgcccccgacggcggctggctgctgctcctgcgtatccaccacctcgtccaggaccacaccaccttcgacgtggtcctcgatgagctgcgcgccgttctcaccggccgcgccgaggaactgccgccgccgctgccgtaccgcgagttcgtcgcgcgggcccggctcggggtctccgacgaggagcacgagcggtacttcgccgacctgctcggcgacgtcaccgagaccaccgccccctacggcctcaccgacacccaccacgacggcaccaccgccgtccaggtgcagctcaccgccgaccgggcgctcaccgcccgggtccgggacctggcccgggcgtacgcggtcagcccggccacgatcctccacctggcctgggcccggctgctcggcacgctcgccggccgcgacgacgtggtcttcggcaccatcctcttcggccggatgaacgccggcgccggcgccgaccgggcccccggcctgttcatcaacacgctgcccgtgcgggtacggctcgcccggcacggcgtcgccgacgcgctgaccgccctgcgcgaccagctcgccgacctgctcgtgcacgagcacgcccccctcgcgctcgcccagcgcgccagcggcgtccccgccgccgccccgctgttcacctcgctgttcaactaccggcacaacctgcccaccgacgggcacccgggcgccggcctggacggggtcacggcggtcctgcaccgggacaacaccaactacccgatggtcgtctccgtcgacgacgacggcacggacttcgcgctggtcgtcgaggcggtcgccccggcggacccgacccgcgtcggttcgctgctgctcacctgcctggacagcctcaccgccgcgctggagcaggcgccgcagaccccgctcggcgccgtggaggtgctggacgccgccgagctgcgccaggtggtcgaggagtggaacgccaccgaggcgccggtcaccgacgagccggtgcccgcggcgttcgcccggcgggtggccgccgacccg

SEQ ID NO: 135 - protein, 466 aa, Micromonospora chersina strain ATCC 53710
ADVYPLTPLQEGILFHHRMADQGGTDVYAMPFVLRADTRQRVDEFLDALRRVVDRHDVYRTAIVWENLREPVQVVWRRADLPVTEVTLEPGGDPVDQLRAAAGGWLELHRAPLIGAHVAADPSGDGWLVLLRMHHLVQDHTSLEVVLDEIRTLLADRADRADRLPAPLPFREFVAHSRHGVPETAHREFFAGLLGDVTETTAPYGLLDVHGDGTDAAQAQLAVTDDLAGRIRTLARTLGVSPATLFHLAWARVVAAVSGRDDVVFGTMLLGRLFAGPGADRAPGLFMNMLPVRARLAGRTVRDALGEMRRQLADLLTHEHAPLVLAQQASALPGGSPLFTSIFNYRHNQVDVRRSGTGIDGVDALLTKEPTNYPLDVSVNQGPDGFEVIVEATAPADPAEVCRLLVTCLANLVAALEHAPDTPIAAVDPLGPAQLDRILREWNATAVPVPDGLVPALFSAQATRTP

SEQ ID NO: 136 - DNA, 1398 bp, Micromonospora chersina strain ATCC 53710
gcagacgtctacccgctcaccccgctccaggagggcatcctcttccaccaccggatggccgaccagggcggcaccgacgtctacgccatgcccttcgtgctccgcgccgacacccggcagcgggtcgacgagttcctcgacgcgctgcgtcgggtcgtcgacaggcacgacgtctaccgcaccgcgatcgtctgggagaacctgcgcgagcccgtccaggtcgtgtggcgccgcgccgacctgccggtcaccgaggtgaccctggaacccggcggcgaccccgtcgaccagcttcgcgccgccgccggcggctggctggagctgcaccgcgcaccgctgatcggcgcgcacgtcgccgcggacccgtccggcgacggctggctcgtgctgctgcgcatgcaccacctcgtgcaggaccacacctccctggaggtggtgctcgacgagatccgcaccctcctggccgaccgcgccgaccgcgccgaccggctgcccgcgccgctgccgttccgcgagttcgtcgcccactcccggcacggcgtgcccgagacggcgcaccgggagttcttcgccgggctcctcggcgacgtcaccgagaccaccgccccctacggcctgctggacgtgcacggggacggcaccgacgccgcgcaggcgcagctcgccgtgaccgacgacctcgccgggcggatccgcaccctggcccggacgctcggcgtcagccccgcgacgctgttccacctggcctgggcgcgggtggtggccgcggtgtccggccgcgacgacgtcgtcttcggcaccatgctgctcggccggctgttcgccggccccggcgccgaccgggcccccggcctgttcatgaacatgctgcccgtccgcgcccggctcgccggccggaccgtccgcgacgcgctgggggagatgcgccggcaactcgccgacctgctcacccacgagcacgcgccgctggtcctcgcccagcaggccagcgccctgcccggcggcagcccgctgttcacgtcgatcttcaactaccggcacaaccaggtcgacgtccgacgctccggcaccggcatcgacggggtcgacgcgctgctcaccaaggaacccaccaactatcccctcgacgtgtcggtcaaccagggcccggacggcttcgaggtgatcgtcgaggccaccgcccccgccgacccggccgaggtctgccggctcctggtcacctgcctggccaaccttgtcgccgccctcgaacacgccccggacacccccatcgccgcggtcgacccgctcggccccgcgcagctcgaccgcatcctgcgcgagtggaacgccaccgccgtgccggtgccggacggcctcgtgcccgcgctgttcagcgcccaggccacccgcacaccg

SEQ ID NO: 137 - protein, 460 aa, Micromonospora chersina strain ATCC 53710
ADIYPLAPFQEGLLLHHLTRGEHEVDVYLSSAVLRFDSRPALDGFLDAFQRVVDRHDIYRTAIVWEGLPEPVQVVLRAARLPVEETVLDPAGGDPADQLMAAGLPRLPLDRAPLLRFRVAAEPGTDRWLALWQVHHLVRDRTTTEVLFAELRAFQAGRGDDLPAPLPFRDFVARLRAGASRAEQEAHFAALLGDLTETTAPYGLVDVHGDGSGITRAHTVIDGPAAARVREVARARNLSPATVLHLAWARVLGAVSGRDDVVFGTVLFGRMQAGAGADRISGPFLNTLPVRVRLRGTVADALTALRDQLAELLVHEHAPLAVAQQASGVRGGSPLVTSVFNYRHGATVQEADVDLPGVELLTAEERTHFPLNVSVGDLDRGFAVTVDAVAPAEPARIAALLETALDGLVTALADAPDTPLPAVPVLDAEFRRRVLVEWNDTAADLGAQTVPARFAARVAA

SEQ ID NO: 138 - DNA, 1380 bp, Micromonospora chersina strain ATCC 53710
gccgacatctacccgctcgcccccttccaggagggcctcctcctgcaccacctcacccgcggcgagcacgaggtggacgtctacctctcctcggccgtgctccgcttcgactcccggcccgcgctcgacggcttcctcgacgcgttccagcgggtggtcgaccggcacgacatctaccgcacggccatcgtctgggaggggctgcccgagcccgtccaggtggtgctccgcgcggcccggctcccggtcgaggagaccgtcctcgacccggccggcggcgaccccgccgaccagctcatggccgccggcctgccccggctgccgctcgaccgggccccgctcctgcggttccgggtcgccgccgagccgggcaccgacaggtggctggcgctgtggcaggtgcaccacctggtccgcgaccggaccaccaccgaggtgctcttcgccgagctgcgggcgttccaggccggacgcggcgacgacctgccggcgccgctgccgttccgggacttcgtggcccggctgcgcgccggcgccagccgggccgagcaggaggcgcacttcgccgcgctgctcggcgacctcaccgagaccaccgccccgtacgggctggtcgacgtgcacggcgacggcagcggcatcacccgcgcgcacaccgtcatcgacggcccggccgccgcccgcgtccgcgaggtggcccgcgcccggaacctcagccccgccaccgtcctgcacctcgcctgggcgcgggtgctcggcgccgtctccggccgggacgacgtggtcttcggcaccgtcctgttcggccggatgcaggccggcgccggcgccgaccggatctccgggccgttcctcaacaccctgcccgtgcgggtccgcctgcgcggcaccgtcgccgacgccctcaccgcgctgcgcgaccagctcgccgagctgctggtccacgaacacgccccgctcgccgtcgcccagcaggccagcggggtacgcggcggcagcccgctggtcacctcggtcttcaactaccggcacggcgccaccgtgcaggaggccgacgtcgacctgcccggcgtcgagctgctcaccgccgaggagcgcacccacttcccgctcaacgtctccgtcggcgacctcgaccgcggcttcgcggtcaccgtcgacgcggtcgccccggccgagccggcccggatcgccgccctgctggagaccgccctggacggcctggtcaccgcgctggccgacgcgccggacacgccgctgccggccgtcccggtgctcgacgccgagttccgccggcgggtgctggtggagtggaacgacaccgccgccgacctgggcgcgcagaccgtgcccgcccggttcgcggcgcgggtggcggcc

SEQ ID NO: 139 - protein, 474 aa, Artificial sequence
  Dual condensation-epimerization domain in NRPS system (1)..(474): Consensus sequence generated from sequences representing dual C-E domains from organisms Actinoplanes sp., Pseudomonas syringae pv. syringae, Ralstonia solanacearum, S. aizunensis, S. griseofuscus, Kitasatosporia sp., Streptomyces sp., S. viridifaciens, Actinomadura sp., and Micromonospora chersina
ADIYPLAPLQEGILFHHLLADGGEDDAYVLPAVLEFDSRERLDAFLGALQQVIDRHDILRTAVVWEGLREPVQVVWRHAELPVEEVTLDPAGIAADPVAQLDAAAGLRMDLGRAPLLRLHVAADPGGGRWLALLRFHHLVQDHTALEVLLAEIQAFLAGRGDELPEPLPFRNFVAQARLGVSRAEHERFFAELLGDVTEPTAPFGLLDVRGDGSGVEEARLPLDAELAARLREQARRLGVSPATLFHLAWARVLGAVSGRDDVVFGTVLFGRMQAGAGADRVPGLFINTLPVRVRLGGQGVLDAVRAMRAQLAELLEHEHAPLALAQRASGVPAPTPLFTSLLNYRHSAVAAVSAEALAAWAGAELEGIRLLSSRERTNYPLTVSVDDLGDGFSLTVQAVAPIDAERVCALLHTALENLVDALEQAPDTPLSAVDVLPAAERRRLLVEWNDTAADYVPAATVPELFEAQVARTP